(19) United States
(12) Patent Application Publication
(10) Pub. No.: US 2023/0399708 A1
(43) Pub. Date: Dec. 14, 2023

(54) Compositions and Methods for Detecting Influenza A, Influenza B, and SARS-CoV-2

(71) Applicant: GEN-PROBE INCORPORATED, SAN DIEGO, CA (US)

(72) Inventors: Craig Brian CLARK, San Diego, CA (US); Jimmykim PHAM, San Diego, CA (US); Paul M. DARBY, San Diego, CA (US); Yi-Hsuan Elisha HO, San Diego, CA (US)

(21) Appl. No.: 18/251,066

(22) PCT Filed: Oct. 29, 2021
(86) PCT No.: PCT/US2021/057223

§ 371 Date: Apr. 28, 2023

Related U.S. Application Data
(60) Provisional application No. 63/107,779, filed on Oct. 30, 2020.

(51) Int. Cl. C12Q 1/70; C12Q 1/6851

(52) U.S. Cl. CPC C12Q 1/701; C12Q 1/6851; C12Q 2600/16

(57) ABSTRACT

Disclosed are nucleic acid oligonucleotides, including primers, probes, and target capture oligonucleotides, for detection of SARS-CoV-2, Influenza A, and Influenza B. Also disclosed are methods of specific nucleic acid amplification and detection using the disclosed oligonucleotides. Corresponding formulations, reaction mixtures, and kits and related methods for preparing aqueous reaction mixtures from dried formulations are described.

### CROSS REFERENCE TO RELATED APPLICATION

This application claims the benefit of U.S. Provisional Application No. 63/107,779, filed Oct. 30, 2020, which is incorporated herein by reference.

### SEQUENCE LISTING

The Sequence Listing written in file 565687_SeqList.txt is 52 kilobytes in size, was created Oct. 29, 2021, and is hereby incorporated by reference

### BACKGROUND

Coronaviruses are a family of RNA viruses that infect avians and mammals, including humans. Coronaviruses belong to the family Coronaviridae, which has four main sub-groupings, known as alphacoronavirus, betacoronavirus, gammacoronavirus, and deltacoronavirus. Human coronaviruses include alphacoronaviruses 229E and NL63 and betacoronaviruses OC43, HKU1, SARS-CoV (the coronavirus that causes severe acute respiratory syndrome, or SARS), SARS-CoV-2 (previously 2019-nCoV or Wuhan CoV), and MERS-CoV (the coronavirus that causes Middle East Respiratory Syndrome, or MERS).

Influenza viruses (types A, B, and C) are members of the orthomyxoviridae family that cause influenza. Type A influenza viruses infect birds and mammals, including humans, whereas types B and C infect humans. Combinations of envelope proteins hemagglutinin (HA) and neuraminidase (NA) subtypes are used to characterize viral isolates. The common nomenclature for HA and NA uses the first letter of the gene followed by the subtype number (e.g., H #N # where # is a number). Influenza B viruses are not categorized into subtypes, but instead are divided into two families, Yamagata and Victoria. Human influenza viruses produce highly contagious, acute respiratory disease that results in significant morbidity and economic costs, with significant mortality among very young, elderly, and immuno-compromised subpopulations. Analysis of human influenza virus A infections has shown that a few HA and NA combinations were clinically significant in causing pandemics during the 1900s, i.e., HINT in 1918, H2N2 in 1957, and H3N2 in 1968.

SARS-CoV-2 can cause severe lower respiratory tract infections (COVID-19) and was declared a global emergency by the World Health Organization. SARS-CoV-2 is responsible for the recent pneumonia outbreak that started in early December 2019 in Wuhan City, Hubei Province, China (Huang et al., Lancet (2020) v395, issue 10223, p. 497).

Due to a high mutation rate, Influenza epidemics occur yearly. Although both types A and B circulate in the population, type A is usually dominant.

The symptoms for the influenza virus infection and coronavirus infection may appear similar. Proper diagnosis and identification of infection is useful in determining the proper course of treatment. There is a need for a test that provides rapid, sensitive, and specific detection influenza A and B viruses and coronavirus SARS-CoV-2 with a minimum of exposure of technical personnel to infectious agents, so that diagnosis is completed in sufficient time to permit effective contact tracing and therapeutic treatment of an infected person.

### SUMMARY

Described are oligonucleotides, compositions, formulations, kits, and methods for amplification and detection of SARS-CoV-2, influenza virus A, and/or influenza virus B. The described oligonucleotides, compositions, formulations and kits can be used to detect the presence or absence of SARS-CoV-2, influenza virus A, and/or influenza virus B in a sample.

The described oligonucleotides, compositions, formulations, kits, and methods can be used for isothermal amplification of target nucleic acids in SARS-CoV-2, influenza virus A, and/or influenza virus B. The isothermal reaction can be, but is not limited to, transcription-mediated amplification (TMA).

The compositions, formulations, or kits generally include (a) at least one primer set comprising first and second SARS-CoV-2-specific primers capable of amplifying a target region of a SARS-CoV-2 nucleic acid, (b) at least one primer set comprising first and second influenza A-specific primers capable of amplifying a target region of an influenza A nucleic acid, and (c) at least one primer set comprising first and second influenza B-specific amplification oligomers capable of amplifying a target region of an influenza B nucleic acid. In some embodiments, the primer sets comprise two second primers for use in amplifying a SARS-CoV-2, Influenza A, and/or Influenza B target sequence. In some embodiments, the compositions, formulations, or kits further include one or more probes for detecting each of SARS-CoV-2, Influenza A, and/or Influenza B. In some embodiments, the compositions, formulations, or kits further comprise one or more target capture oligonucleotides (TCO) for separating SARS-CoV-2, Influenza A, and/or Influenza B target nucleic acid(s) from other components of a sample.

The described oligonucleotides, compositions, and formulations can also be provided as dried or lyophilized powders or cakes. In some embodiments, kits comprising such dried or lyophilized powders or cakes for amplifying one or more of SARS-CoV-2, influenza A, and influenza B nucleic acids are described. In some embodiments, methods for preparing an aqueous reaction mixture for determining the presence or absence of one or more of SARS-CoV-2, influenza A, and influenza B in a sample are provided. The methods generally include the step of combining the dried composition with an aqueous reconstitution reagent.

The described oligonucleotides, compositions, and formulations can be provided as aqueous solutions. In some embodiments, kits comprising such aqueous solutions, for amplifying one or more of SARS-CoV-2, influenza A, and influenza B nucleic acids are described.

In some embodiments, methods for determining the presence or absence of one or more of SARS-CoV-2, influenza A, and influenza B in a sample are described. The methods generally include performing an in vitro nucleic acid amplification reaction utilizing any of the described compositions, formulations, or kits to generate amplicons corresponding to one or more SARS-CoV-2, influenza A, and influenza B target sequences, and detecting the presence or absence of the one or more SARS-CoV-2, influenza A, and influenza B amplicons.

In some embodiments, the described oligonucleotides, compositions, formulations, and kits are suitable for use in amplifying and/or detecting one or more of SARS-CoV-2, Influenza A, and/or Influenza B in multiplex amplification and/or detection reactions. The multiplex reactions can be used to detect the presence or absence one or more of SARS-CoV-2, Influenza A, and/or Influenza B in a sample. For example, the amplification systems disclosed herein can be used to amplify and optionally detect SARS-CoV-2 or Influenza A or Influenza B, SARS-CoV-2 and Influenza A or Influenza B, Influenza A and SARS-CoV-2 or Influenza B; Influenza B and Influenza A or SARS-CoV-2; or SARS-CoV-2, Influenza A, and Influenza B, The multiplex amplification reaction can be a transcription-mediated amplification (TMA) reaction

In some embodiments, the described oligonucleotides, compositions, formulations, and kits are suitable for use in amplifying one or more of SARS-CoV-2, Influenza A, and/or Influenza B in a biphasic amplification reaction. The biphasic amplification reaction can be a TMA reaction.

In some embodiments, amplifying the target sequence of influenza virus A uses at least one oligonucleotide selected from sequences consisting of SEQ ID NO: 1-5 and one oligonucleotide selected from sequences consisting of SEQ ID NO: 28-33 or at least one oligonucleotide selected from sequences consisting of SEQ ID NO: 6-11 and one oligonucleotide selected from sequences consisting of SEQ ID NO: 34-27.

In some embodiment, the step of amplifying the target sequence of influenza virus B uses at least one oligonucleotide selected from sequences consisting of SEQ ID NO: 72-75 and one oligonucleotide selected from sequences consisting of SEQ ID NO: 96-99 or at least one oligonucleotide selected from sequences consisting of SEQ ID NO: 77-78 and one oligonucleotide selected from sequences consisting of SEQ ID NO: 92-95.

In some embodiments, the step of amplifying the target sequence of SARS-CoV-2 uses at least one oligonucleotide selected from sequences consisting of SEQ ID NO: 126 and one oligonucleotide selected from sequences consisting of SEQ ID NO: 167-169 or at least one oligonucleotide selected from sequences consisting of SEQ ID NO: 127-131 and one oligonucleotide selected from sequences consisting of SEQ ID NO: 170-181 or at least one oligonucleotide selected from sequences consisting of SEQ ID NO: 132-134 and one oligonucleotide selected from sequences consisting of SEQ ID NO: 182-194.

In some embodiments, the detecting step for detecting an Influenza A amplicon uses at least one probe selected from the sequences consisting of SEQ ID NO: 54-59, and SEQ ID NO: 60-62.

In some embodiments, the detecting step for detecting an Influenza B amplicon uses at least one probe selected from the sequences consisting of SEQ ID NO: 114-116, and SEQ ID NO: 117-119.

In some embodiments, the detecting step for detecting a SARS-CoV-2 amplicon uses at least one probe selected from the sequences consisting of SEQ ID NO: 232-234, SEQ ID NO: 266-275, SEQ ID NO: 235-240 SEQ ID NO: 257, and SEQ ID NO: 241-243.

In some embodiments, a kit further contains primers and at least one probe for amplification and detection of an internal control. In some embodiments, the method includes the steps of providing an internal control target nucleic acid, amplifying a target sequence contained in the internal control target nuclei acid, and detecting the corresponding amplicon, thereby indicating that the amplifying and detecting steps of the method were properly performed, and any reagents and equipment functioned properly.

In some embodiments, one or more of the primers, probes or TCOs contains at least one modified nucleotide. The at least one modified nucleotide can be, but is not limited to a 2′-methoxy nucleotide, a 2′ fluoro nucleotide or a locked nucleic acid (LNA) nucleotide.

Detecting the presence or absence of SARS-CoV-2, influenza A, and/or influenza B in a sample can be used to diagnose SARS-CoV-2, influenza A, and/or influenza B infection, identify a subject infected with SARS-CoV-2, influenza A, and/or influenza B infection to aid in contact tracing, or to guide antiviral treatment.

### DESCRIPTION

### I. Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise. For example, “a nucleic acid” is understood to represent one or more nucleic acids. As such, the terms “a” (or “an”). “one or more,” and “at least one” can be used interchangeably herein.

In general, the term “about” indicates variation in a quantity of a component of a composition not having a significant effect on the activity or stability of the composition. The term “about” means within an acceptable error range for the particular value as determined by one of ordinary skill in the art, which will depend in part on how the value is measured or determined. For example, “about” can mean within 1 standard deviation or per the practice in the art. When a value is expressed as “about” X or “approximately” X, the stated value of X will be understood to be accurate to +20%, +10%, or +5%.

All ranges are to be interpreted as encompassing the endpoints in the absence of express exclusions such as “not including the endpoints.” One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 10.35, etc.).

A “sample” includes any specimen that contains or is suspected of containing one or more of SARS-CoV-2, influenza A, and influenza B, including components thereof, such as nucleic acids or fragments of nucleic acids. Samples include “biological samples” which include any tissue or material derived from a living or dead mammal (such as a human) or organism. Biological samples include, but are not limited to, nasopharyngeal swab, nasal swab, mid-turbinate swab, oropharyngeal swab, throat swab, nasal wash, bronchial wash, nasal aspirate, sputum, blood, plasma, serum, blood cells, saliva, mucous, respiratory tissue, exudates (e.g., bronchoalveolar lavage), sputum, tracheal aspirates, lymph node, gastrointestinal tissue, feces, urine, genitourinary fluid, and biopsy cells or tissue. A sample may be treated or processed by sample preparation. A sample may be an individual sample (i.e., a sample derived from a single subject) or a pooled sample (i.e., a sample prepared by pooling a plurality of individual samples).

“Sample preparation” refers to any steps or methods required to prepare a sample for amplification and/or detection. A sample may be treated chemically, physically, and/or mechanically to disrupt tissue, cells, or cellular components to release intracellular components into a aqueous or organic solution which may further contain enzymes, buffers, salts, detergents and the like, which are used to prepare a biological sample for analysis. A sample may also be treated chemically, physically, and/or mechanically to remove cellular components or debris. A sample may be processed by passing the samples over or through a filtering device, centrifugation, or by adherence to a medium, matrix, or support. Sample preparation includes knowns method of concentrating components, such as polynucleotides, from a larger sample volume, such as by filtration from larger volume sample, centrifugation, or by isolation of microbes from a sample by using standard microbiology methods. Sample preparation may also include use of a polynucleotide to specifically or non-specifically capture a target nucleic acid and separate it from other sample components (e.g., as described in U.S. Pat. No. 6,110,678 and International Patent Application Pub. No. WO 2008/016988, each incorporated by reference herein).

“Separating” or “purifying” refers to removal of one or more components of a mixture, such as a sample, from one or more other components in the mixture. Sample components include nucleic acids, cellular fragments, proteins, carbohydrates, lipids, and other compounds. Separating or purifying does not connote any particular degree of purification. In some embodiments, at least 70%, at least 75%, at least 80%, at least 85%, at least 90%, or at least 95%, of the target nucleic acid or amplified product is separated or removed from other components in the mixture.

A “nucleotide” is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose.

“Nucleic acid” and “polynucleotide” refer to a multimeric compound comprising nucleotides and/or nucleotide analogs linked together to form a biopolymer. The biopolymers include conventional RNA, conventional DNA, mixed RNA-DNA, and nucleotide analog containing versions thereof. A nucleic acid “backbone” may be made up of a variety of linkages, including one or more of sugar-phosphodiester linkages, peptide-nucleic acid bonds (“peptide nucleic acids” or PNA), phosphorothioate linkages, methylphosphonate linkages, or combinations thereof. Sugar moieties of a nucleic acid may be ribose, deoxyribose, or similar compounds with substitutions or modifications, e.g., analogs with a methoxy, fluoro or halide group at the 2′ position of the ribose (also referred to herein as “2′-O-Me” or “2′-methoxy” or 2′-fluoro, or “2′-halide”). Nitrogenous bases may be conventional bases, adenine (A), uracil (U), guanine (G), thymine (T), and cytosine (C), and analogs thereof (e.g., inosine, 5 methyl 2′ deoyxcytosine (“5-Me-dC” or “5MeC”), and isoguanine). Nucleic acids may include one or more “abasic” residues where the backbone includes no nitrogenous base for position(s) of the polymer.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “complementarity” refers to the ability of a polynucleotide to form hydrogen bond(s) (hybridize) with another polynucleotide sequence by either traditional Watson-Crick base pairing or other non-traditional types of base paring. The two complementary polynucleotide strands are antiparallel one another. A percent complementarity indicates the percentage of bases, in a contiguous strand, in a first nucleic acid sequence which can form hydrogen bonds (e.g., Watson-Crick base pairing) with a second nucleic acid sequence (e.g., 6, 7, 8, 9, 10 out of 10 being 50%, 60%, 70%, 80%, 90%, and 100% complementary). Percent complementarity is calculated in a similar manner to percent identify.

By “RNA equivalents” and “DNA equivalents” is meant RNA and DNA molecules having essentially the same nucleic acid sequence or complementary base pair hybridization properties. RNA and DNA equivalents have different sugar moieties (i.e., ribose versus deoxyribose) and may differ by the presence of uracil in RNA and thymine in DNA. The differences between RNA and DNA equivalents do not contribute to differences in homology because the equivalents have the same degree of complementarity to a particular sequence. By “DNA/RNA chimeric” is meant a nucleic acid comprising both DNA and RNA nucleotides. Unless the context clearly dictates otherwise, reference to a nucleic acid includes the RNA and DNA equivalents and DNA/RNA chimerics thereof.

An “oligomer”, “oligonucleotide”, or “oligo” is a polymer made up of two or more nucleoside subunits or nucleobase subunits coupled together. An oligomer refers to a nucleic acid of generally less than 1,000 nucleotides (nt), including those in a size range having a lower limit of about 5 nt and an upper limit of about 900 nt. In some embodiments, the oligomers are in a size range having a 5 to 15 nt lower limit and a 50 to 500 nt upper limit. In some embodiments, the oligomers are in a size range of 10-100 nucleobases, 10-90 nucleobases, 10-nucleobases, 10-70 nucleobases, or 10-60 nucleobases. The oligonucleotide may be DNA and/or RNA and/or analogs thereof. The term oligonucleotide does not denote any particular function to the reagent; rather, it is used generically to cover all such reagents described herein. Oligomers can be made synthetically by using any well-known in vitro chemical or enzymatic method, and may be purified after synthesis by using standard methods, e.g., high-performance liquid chromatography (HPLC). Standard phosphoramidite solid phase chemistry is often used to prepare oligonucleotides (see e.g., Caruthers et al., Methods Emzymol., 154:287 (1987)). Automated solid-phase chemical synthesis using cyanoethyl phosphoramidite precursors has been described by Barone (see Barone et al., Nucleic Acids Res., 12(10):4051 (1984)). Batt discloses a procedure for synthesizing oligonucleotides containing phosphorothioate linkages in U.S. Pat. No. 5,449,769, and Riley et al. disclose the synthesis of oligonucleotides having different linkages including methylphosphonate linkages in U.S. Pat. No. 5,811,538. Moreover, methods for the organic synthesis of oligonucleotides are known to those of skill in the art and are described in, for example, Sambrook et al., Molecular Cloning, A Laboratory Manual, 2nd ed. (Cold Spring Harbor Laboratory Press, Cold Spring Harbor, N Y, 1989), ch. Described are oligomers that include RNA polymerase promoter-containing oligomers (also termed promoter primers; e.g., T7 primers), non-RNA polymerase promoter-containing oligomers (also termed non-T7 primers, NT7 primers, or non-promoter primers), probe oligomers (also termed detection oligomers or detection probes, probes, or Torches), target capture oligomers (TCOs), forward primers, and reverse primers.

References to “the sequence of SEQ ID NO: X” refer to the sequence of nucleobases, nucleotides and/or nucleotide analogs linked together to form a biopolymer. Reference to a sequence by SEQ ID NO: does not connote the identity of the backbone (e.g., RNA, 2′-O-Me RNA, or DNA) or any nucleobase modifications (e.g., methylation of cytosine residues (“5MeC”)) unless the context clearly dictates otherwise. Unless the context clearly dictates otherwise, reference to a sequence by SEQ ID NO: includes reference to its complementary sequence (e.g., reference to the sequence 5′-ttagc-3′ includes reference to the sequence 5′-gctaa-3′).

The term “target capture” refers to selectively separating or isolating a target nucleic acid from other components of a sample mixture, such as cellular fragments, organelles, proteins, lipids, carbohydrates, or other nucleic acids. A target capture system may be specific and selectively separate a predetermined target nucleic acid from other sample components (e.g., by using a sequence specific to the intended target nucleic acid, such as a TCO TS sequence). Target capture methods and compositions have been previously described in detail (U.S. Pat. Nos. 6,110,678 and 6,534,273; and US Pub. No. 2008/0286775 A1). In some embodiments, target capture utilizes a TCO in solution phase and an immobilized capture probe attached to a support to form a complex with the target nucleic acid and separate the captured target from other components.

A “Target capture oligonucleotide” (TCO) is a nucleic acid oligonucleotide that specifically hybridizes to a sequence in a target nucleic acid by standard base pairing and joins to a binding partner on an immobilized probe to capture the target nucleic acid to a support. TCOs can be used to capture or isolate the target nucleic acid from a sample. The TCO comprises a target specific (TS) nucleotide sequence that hybridizes to (i.e., is complementary to) a region of a target nucleic acid. In some embodiments, the TCO TS sequence comprises a nucleotide sequence having at least 90%, at least 95%, or 100% complementarity to a nucleotide sequence present in the target nucleic acid and hybridizes to a region in the target nucleic acid sequence (a TCO binding site). A TCO includes an immobilized capture probe-binding region that binds to an immobilized capture probe (e.g., by specific binding pair interaction). In some embodiments, the TCO TS sequence is linked to the capture probe-binding region. In some embodiments, the TCO TS sequence and capture probe-binding region are present on two different oligonucleotides joined together by one or more linkers. In some embodiments, the capture probe-binding region comprises: a poly A sequence, a poly T sequence, or a polyT-polyA sequence. In some embodiments, a polyT-polyA sequence comprises (dT)₀₋₃(dA)₁₄₋₃₀ or (dT)₃(dA)₃₀.

An “immobilized capture probe” provides a means for joining a TCO to a solid support. In some embodiments, an immobilized capture probe contains a base sequence recognition molecule joined to the solid support, which facilitates separation of bound target polynucleotide from unbound material. Any known solid support may be used, such as matrices and particles free in solution. For example, solid supports may be nitrocellulose, nylon, glass, polyacrylate, mixed polymers, polystyrene, silane polypropylene and magnetically attractable particles. In some embodiments, the supports include magnetic spheres that are monodisperse (i.e., uniform in size about 5%). The immobilized capture probe may be joined directly (e.g., via a covalent linkage or ionic interaction), or indirectly to the solid support. Common examples of useful solid supports include magnetic particles or beads.

A “target nucleic acid” is a nucleic acid comprising a target sequence to be amplified and/or detected. Target nucleic acids may be DNA or RNA and may be either single-stranded or double-stranded. A target nucleic acid can be, but is not limited to, a genomic nucleic acid, a transcribed nucleic acid, such as an mRNA. For a single stranded target nucleic acid, such as a single stranded RNA virus or an mRNA, the target nucleic acid includes the complement thereof. A target nucleic acid can also be a nucleic acid derived from a genomic or transcribed nucleic acid. A target nucleic acid (including where appropriate its complement) contains sequences that hybridize to capture oligonucleotides, primers, and/or probes used to amplify and/or detect the target nucleic acid. The target nucleic acid may include other sequences besides the target sequence which may not be amplified. A “target sequence” or “target nucleic acid sequence” is the particular nucleotide sequence of the target nucleic acid that is to be amplified and/or detected. The target sequence, which includes a complement thereof, contains sequences that hybridize to primers and probes used to amplify and/or detect the target nucleic acid.

A “target hybridizing sequence,” “target hybridizing region,” or “target specific sequence” is a sequence in an oligonucleotide that hybridizes to a region in a target nucleic acid. The target hybridizing region is a contiguous sequence of nucleotides that hybridizes to a complementary contiguous sequence of nucleotides in the target nucleic acid sequence. Target hybridizing sequences are configured to specifically hybridize with a target nucleic acid. Target hybridizing sequences may be 100% complementary to the portion of the target nucleic acid to which they are configured to hybridize, but not necessarily. Target hybridizing sequences may include inserted, deleted, and/or substituted nucleotide residues relative to a target sequence provided the target hybridizing sequence specifically hybridizes with a target nucleic acid. A primer or probe can contain both target specific sequence and non-target specific sequence. The target specific sequence (or target hybridizing sequence or target hybridizing region) is the portion of the oligonucleotide that is configured to hybridize with a target nucleic acid. Reference to an oligonucleotide (such as a primer or probe) comprising a target hybridizing sequence consisting of SEQ ID NO: X indicates the portion of the oligonucleotide that is complementary to the target nucleic acid consists only of the indicated SEQ ID NO. The oligonucleotide may contain other non-target hybridizing sequences or other components (such as a label), but the target hybridizing sequence consists of the sequence in the indicated SEQ ID NO.

“Non-target specific sequence” or “non-target hybridizing sequence” refers to a region of an oligomer sequence, wherein said region does not stably hybridize with a target nuclei acid under standard hybridization conditions. Oligomers with non-target specific sequences include, but are not limited to, promoter primers, certain target capture oligomers, and certain probes, such as torches and molecular beacons. In a probe oligonucleotides a non-target hybridizing sequence can hybridize with other nucleotides in the probe oligonucleotides to for a stem region. In a promoter primer, a non-target hybridizing sequence can comprise an RNA promoter sequence. In a target capture oligonucleotide, a non-target hybridizing sequence can hybridize with a complementary sequence linked to a solid support.

“Target a sequence,” is used in reference to a region of a target sequence and refers to a process whereby an oligonucleotide hybridizes to the target sequence in a manner that allows for amplification and/or detection as described herein.

The term “configured to specifically hybridize to” indicates that the target hybridizing region of a primer, probe, or other oligonucleotide is designed to have a polynucleotide sequence that can target a sequence of the referenced target sequence. The oligonucleotide is designed to function as a component of an assay for amplification and/or detection of the target sequence from a sample, and therefore is designed to target the target sequence in the presence of other nucleic acids found in testing samples. “Specifically hybridize to” does not mean exclusively hybridize to, as some small level of hybridization to non-target nucleic acids may occur, as is understood in the art. Rather, “specifically hybridize to” means that the oligonucleotide is configured to function in an assay to primarily hybridize the target so that an accurate amplification and/or detection of target nucleic acid in a sample can be determined.

An “amplification oligonucleotide,” “amplification oligomer,” or “primer” is an oligonucleotide that hybridizes to a target nucleic acid and participates in a nucleic acid amplification reaction, e.g., serving as a primer. Amplification oligomers can have 3′ ends that are extended by polymerization as part of the nucleic acid amplification reaction. Amplification oligomers that provide both a 3′ target hybridizing region that is extendable by polymerization and a 5′ promoter sequence are referred to as promoter primers. Amplification oligomers may be optionally modified to include 5′ non-target hybridizing regions such as tags, promoters (as mentioned), or other sequences used or useful for manipulating or amplifying the primer or target oligonucleotide.

“Nucleic acid amplification” refers to any in vitro procedure that produces multiple copies of a target nucleic acid sequence, or its complementary sequence, or fragments thereof (i.e., an amplified sequence containing less than the complete target nucleic acid). Examples of nucleic acid amplification procedures include transcription associated methods, such as transcription-mediated amplification (TMA), nucleic acid sequence-based amplification (NASBA) and others (e.g., U.S. Pat. Nos. 5,399,491, 5,554,516, 5,437,990, 5,130,238, 9,139,870, 4,868,105, and 5,124,246), and polymerase chain reaction (PCR) (e.g., U.S. Pat. Nos. 4,683,195, 4,683,202, and 4,800,159).

“Transcription-mediated amplification” uses a DNA polymerase (e.g., reverse transcriptase), an RNA polymerase, deoxyribonucleoside triphosphates, ribonucleoside triphosphates, primers, including a promoter primer, and optionally may include other oligonucleotides, to produce multiple RNA transcripts from a nucleic acid template (described in detail in U.S. Pat. Nos. 5,399,491 and 5,554,516, Kacian et al., U.S. Pat. No. 5,437,990, Burg et al., PCT Nos. WO 88/01302 and WO 88/10315, Gingeras et al., U.S. Pat. No. 5,130,238, Malek et al., U.S. Pat. Nos. 4,868,105 and 5,124,246, Urdea et al., PCT No. WO 94/03472, McDonough et al., PCT No. WO 95/03430, and Ryder et al., each of which is incorporated herein by reference). Methods that use TMA are described in detail previously (U.S. Pat. Nos. 5,399,491 and 5,554,516, each of which is incorporated herein by reference). TMA can be a substantially isothermal amplification. TMA can also be run as a biphasic amplification reaction.

The term “substantially isothermal amplification” refers to an amplification reaction that is conducted at a substantially constant temperature. The isothermal portion of the reaction may be preceded or followed by one or more steps at a variable temperature, for example, a first denaturation step and a final heat inactivation step or cooling step. It will be understood that this definition does not exclude small variations in temperature but is rather used to differentiate the isothermal amplification techniques from other amplification techniques known in the art that basically rely on “cycling temperatures” in order to generate the amplified products.

An “amplicon” or “amplification product” is a nucleic acid molecule generated in a nucleic acid amplification reaction and which is derived (amplified) from a target nucleic acid. An amplicon or amplification product contains a target nucleic acid sequence that may be of the same and/or opposite sense as the target nucleic acid.

“Relative fluorescence unit” (“RFU”) is a unit of measurement of fluorescence intensity. RFU varies with the characteristics of the detection means used for the measurement and can be used as a measurement to compare relative intensities between samples and controls.

A “detection probe oligomer,” “probe oligonucleotide,” “detection probe,” or “probe” is an oligomer that hybridizes specifically to a target sequence, including an amplified product, under conditions that promote nucleic acid hybridization, for detection of the target nucleic acid. Detection may either be direct (i.e., probe hybridized directly to the target) or indirect (i.e., a probe hybridized to an intermediate structure that links the probe to the target). A probe's target sequence generally refers to the specific sequence within a larger sequence which the probe hybridizes specifically. A detection probe may include target specific sequence(s) and non-target specific sequence(s). Such non-target specific sequences can include sequences which will confer a desired secondary or tertiary structure, such as a hairpin structure, which can be used to facilitate detection and/or amplification. The non-target specific sequence can be located at the 3′ end or 5′ end of the probe target specific sequence. A probe target specific sequence and non-target specific sequence can form a contiguous nucleotide sequence. A non-target specific sequence at one end of a probe (e.g., 3′ end or 5′ end), can be complementary to (i.e., hybridize with) another sequence in the probe, typically at or near the opposite end of the probe from the non-target specific sequence, thereby forming a hairpin or stem-loop structure. The non-target specific sequence can be perfectly complementary to a sequence in the probe or it may have 1-2 mismatches. A non-target specific sequence can be designed to be complementary with another sequence in the probe such that the probe forms an intramolecular double strand region (stem-loop structure) when the probe is not hybridized with a target nucleic acid, but does not form an intramolecular double strand region when the probe is bound to a target nucleic acid. A probe can have a detectable label. The detectable label can be joined directly or indirectly to the probe.

A “Molecular torch” or “Torch” is a type of probe and can be used to indicate whether an amplicon is present in the sample. Molecular torches include distinct regions of self-complementarity. When exposed to the target, the two self-complementary regions (fully or partially complementary) of the molecular torch melt, thus allowing for the individual nucleotides (comprising the target binding domain) to hybridize to the complementary contiguous nucleotides on the target nucleic acid sequence. Molecular torches are designed so that the target binding domain favors hybridization to the target nucleic acid sequence over the target closing domain (region of self-complementarity). The target binding domain and the target closing domain of a molecular torch include interacting labels (e.g., fluorescent dye and quencher, FRET pair), so that a different signal is produced when the molecular torch is self-hybridized, as opposed to when the molecular torch is hybridized to a target nucleic acid sequence (thereby permitting detection of probe:target duplexes in a test sample in the presence of unhybridized probe). Methods of synthesizing labels, attaching labels to nucleic acid, and detecting signals from labels are well known in the art (e.g., Sambrook et al., supra, at Chapter and U.S. Pat. Nos. 5,658,737, 5,656,207, 5,547,842, 5,283,174, and 4,581,333, and EP Pat. App. 0747706).

“Stringent hybridization conditions,” or “stringent conditions” are conditions permitting an oligomer to preferentially hybridize to a target sequence and not to nucleic acid derived from a closely related non-target nucleic acid (i.e., conditions permitting an oligomer to hybridize to its target sequence to form a stable oligomer:target hybrid, but not form a sufficient number of stable oligomer:non-target hybrids, so as to allow for amplification and/or detection of target nucleic acids but not non-targeted organisms). While the definition of stringent hybridization conditions does not vary, the actual reaction environment that can be used for stringent hybridization may vary depending upon factors including the GC content and length of the oligomer, the degree of similarity between the oligomer sequence and sequences of non-target nucleic acids that may be present in the test sample, and the target sequence. Hybridization conditions include the temperature and the composition of the hybridization reagents or solutions. Stringent hybridization conditions are readily ascertained by those having ordinary skill in the art.

A “label” or “detectable label” is a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which amplify a detectable signal). Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g., bioluminescent, phosphorescent, or chemiluminescent compound such as an acridinium ester (“AE”) compound), and fluorescent compound (i.e., fluorophore). A fluorophore may be used in combination with a quencher molecule that absorbs light emitted by the fluorophore when in close proximity to the fluorophore. Detectably labeled probes include, but are not limited to, TaqMan™ probes, AE-labeled probes, molecular torches, and molecular beacons.

A “quencher” is a molecule that absorbs light. Quenchers are commonly used in combination with a light emitting label such as a fluorophore to absorb emitted light when in close proximity to the fluorophore. Quenchers are well-known in the art and include, but are not limited to, Black Hole Quencher™ (or BHQ™, BHQ-1™, or BHQ-2™), Blackberry Quencher, Dabcyl, QSY, and Tamra™ compounds, to name a few.

“Specificity” in the context of an amplification and/or detection system, refers to the characteristic of the system which describes its ability to distinguish between target and non-target sequences dependent on sequence and assay conditions. In terms of nucleic acid amplification, specificity generally refers to the ratio of the number of specific amplicons produced to the number of side-products (e.g., the signal-to-noise ratio). In terms of detection, specificity generally refers to the ratio of signal produced from target nucleic acids to signal produced from non-target nucleic acids.

“Sensitivity” refers to the precision with which a nucleic acid amplification reaction can be detected or quantitated. The sensitivity of an amplification reaction is generally a measure of the smallest copy number of the target nucleic acid that can be reliably detected in the amplification system, and will depend, for example, on the detection assay being employed, and the specificity of the amplification reaction, e.g., the ratio of specific amplicons to side-products.

Any of the described oligonucleotides can contain at least one modified nucleotide. The modified nucleotide can be, but is not limited to, 2′-O-methyl modified nucleotide, 2′-fluoro modified nucleotide, or a 5-methyl cytosine. In some embodiments, an amplification oligonucleotide comprises two or more modified nucleotides. The two or more modified nucleotides may be the same or different. In some embodiments, thymidine nucleotides can be substituted for uridine nucleotides. In some embodiments, all thymidine nucleotides can be substituted for uridine nucleotides. In some embodiments, 5-methyl-2-deoxycytosine bases can be used to increase the stability of the duplex by raising the Tm by about 0.5°-1.3° C. for each 5′methyl-2′deoxycytosine incorporated in an oligonucleotide (relative to the corresponding unmethylated amplification oligonucleotides).

Provided herein are compositions, formulations, kits, and methods for amplifying and/or detecting SARS-CoV-2, influenza A, and/or influenza B nucleic acids in a sample. The compositions, formulations, kits, and methods provide oligonucleotides for amplification and detection of SARS-CoV-2, influenza A and/or influenza B. Other oligonucleotides may be used as TCOs for capture of SARS-CoV-2, influenza A, and/or influenza B target nucleic acid.

The methods provide for the sensitive and specific detection of SARS-CoV-2, influenza A, and/or influenza B nucleic acids in a sample. The methods include performing a nucleic acid amplification of one or more SARS-CoV-2, influenza A, and influenza B target regions, and detecting the amplified product(s) by, for example, specifically hybridizing the amplified product with a probe that provides a signal to indicate the presence of SARS-CoV-2, influenza A, and influenza B in the sample. The amplification step includes contacting the sample with one or more primers specific for a target sequence in a SARS-CoV-2, influenza A, or influenza B target nucleic acid to produce an amplified product if the SARS-CoV-2, influenza A, or influenza B nucleic acid is present in the sample. In some embodiments, detecting the amplified product uses a hybridizing step that includes contacting the amplified product with at least one probe specific for a sequence amplified by the selected amplification primers, e.g., a sequence contained in the target sequence flanked by a pair of selected primers.

In some embodiments, the oligonucleotides are configured to specifically hybridize to SARS-CoV-2, influenza A, or influenza B target nucleic acids with minimal cross-reactivity to one or more pathogens that are not SARS-CoV-2, influenza A, or influenza B. In some embodiments, the oligonucleotides, compositions, and formulations are part of a multiplex amplification system for amplifying and detecting one or more of SARS-CoV-2, influenza A, or influenza B, if present, in a sample in a single reaction.

In some embodiments, methods for utilizing any of the described primer sets, probes, TCOs, compositions, formulations, or kits are provided. Any method disclosed herein is also to be understood as a disclosure of corresponding uses of materials involved in the method directed to the purpose of the method. Any of the oligonucleotides described herein, comprising a SARS-CoV-2, influenza A, or influenza B target hybridizing sequence, and any combinations (e.g., compositions, formulations, and kits) comprising such oligonucleotides are to be understood as also disclosed for use in detecting and/or quantifying SARS-CoV-2, influenza A, and influenza B and for use in the preparation of a composition for detecting and/or quantifying SARS-CoV-2, influenza A, and influenza B.

Broadly speaking, the methods may comprise one or more of the following components: target capture, in which SARS-CoV-2, influenza A, and/or influenza B nucleic acids (e.g., from a sample, such as a clinical sample), if present, are annealed to TCOs; isolation, e.g., washing, to remove material not associated with the TCOs; amplification; and amplicon detection (including amplicon quantification), which may be performed in real time with amplification. Some embodiments involve each of the foregoing steps. In some embodiments, amplification comprises exponential amplification, optionally with a preceding linear amplification step (e.g., biphasic amplification). In some embodiments, amplification comprises exponential amplification and amplicon detection. In some embodiments, amplification and optionally detection comprises any two of the components listed above. In some embodiments, amplification and optionally detection comprises any two components listed adjacently above, e.g., washing and amplification, or amplification and detection.

Amplifying a SARS-CoV-2, an influenza A, or an influenza B target sequence utilizes an in vitro amplification reaction using at least two primers that flank a target region to be amplified. Particularly suitable oligomer combinations for amplification of SARS-CoV-2, influenza A, and influenza B target regions are described herein.

An amplification and/or detection methods in accordance with the present disclosure can further include the step of obtaining the sample to be subjected to subsequent steps of the method. In certain embodiments, “obtaining” a sample to be used includes, for example, receiving the sample at a testing facility or other location where one or more steps of the method are performed, and/or retrieving the sample from a location (e.g., from storage or other depository) within a facility where one or more steps of the method are performed.

### II. Amplification Primers and Detection Probes

In some embodiments, a primer set includes at least two primers configured for amplifying a target nucleic acid sequence. In some embodiments, a primer set includes a first primer (e.g., NT7 primer) and a second primer (e.g., promoter primer). In some embodiments, a primer set includes a first primer and a two second primers. Described below are primer sets and probes for amplifying and detecting a target nucleic acid sequence in a first region (region 1) of Influenza A, a second region (region 2) of Influenza A, a first region (region 1) of Influenza B, a second region (region 2) of Influenza B, a first region (region 1) of SARS-CoV-2, a second region (region 2) of SARS-CoV-2, and third region (region 3) of SARS-CoV-2. The primer sets for amplifying the different regions of the viruses can be used in any combination. In some embodiments, a composition, formulation or kit comprises primers sets and corresponding probes for amplifying and detecting at least two regions of SARS-CoV-2, at least one region of Influenza A, and at least one region of Influenza B in a multiplex reaction.

One skilled in the art will understand that at least one primer comprises a target hybridizing sequence in the sense orientation and at least one primer comprises a target hybridizing sequence in the antisense orientation relative to the target nucleic acid. The primers are configured such that the antisense primer is situated downstream of the sense primer and the sense primer is situated downstream of the antisense primer (i.e., the at least two primers are configured such that they flank the target region to be amplified and prime polymerization in the direction of the other primer, thereby amplifying the region between the two primers).

A. Influenza a Region 1

In some embodiments, an influenza A region 1 first primer comprises a target hybridizing region 15-30 nucleobases in length, wherein the target hybridizing region is contained within SEQ ID NO:15 or an RNA equivalent or a DNA/RNA chimeric thereof and contains the nucleotide sequence of SEQ ID NO:16 or SEQ ID NO:17 or an RNA equivalent or a DNA/RNA chimeric thereof (Table 1a). In some embodiments, a influenza A region 1 first primer comprises a target hybridizing region 15-30 nucleobases in length, wherein the target hybridizing region is contained within SEQ ID NO:15 an RNA equivalent or a DNA/RNA chimeric thereof and contains a nucleotide sequence differing by no more than 0, 1, 2, 3, 4, or nucleotides from the nucleotide sequence of SEQ ID NO:16 or SEQ ID NO:17 or an RNA equivalent or a DNA/RNA chimeric thereof. In some embodiments, the influenza A region 1 first primer comprises the nucleotide sequence of SEQ ID NO: 1, 2, 3, 4, or 5 or an RNA equivalent or a DNA/RNA chimeric thereof. In some embodiments, the influenza A region 1 first primer consists essentially of the nucleotide sequence of SEQ ID NO: 1, 2, 3, 4, or 5 or an RNA equivalent or a DNA/RNA chimeric thereof. In some embodiments, the influenza A region 1 first primer consists of the nucleotide sequence of SEQ ID NO: 1, 2, 3, 4, or 5 or an RNA equivalent or a DNA/RNA chimeric thereof. In some embodiments, the influenza A region 1 first primer comprises a nucleotide sequence having no more than 0, 1, 2, 3, 4, or 5 mismatches from the nucleotide sequence of SEQ ID NO: 1, 2, 3, 4, or 5 or an RNA equivalent or a DNA/RNA chimeric thereof. In some embodiments, the influenza A region 1 first primer comprises a nucleotide sequence having at least 80%, at least 85% at least 90%, or at least 95% homology to the nucleotide sequence of SEQ ID NO: 1, 2, 3, 4, or 5 or an RNA equivalent or a DNA/RNA chimeric thereof. An influenza A region 1 first primer can have 1 or more modified nucleotides or nucleotide analogs. In some embodiments, the influenza A region 1 first primer is an NT7 primer.

TABLE 1a
Influenza A region 1 NT7 primer sequences

| SEQ ID NO: | Sequence (5′→3′) |
|---|---|
| 15 | ggtcttgtctttagccattccatgagagcctcaaga |
| | tc |
| 16 | ctttagccattccat |
| 17 | ccattccatgagagcctc |
| 1 | GGTCTTGTCTTTAGCCATTCCAT |
| 2 | CCATTCCATGAGAGCCTCAAGATC |
| 3 | CTTTAGCCATTCCATGAGAGCCTCAAG |
| 4 | CTTGTCTTTAGCCATTCCATGAGAGCCTC |
| 5 | GGTCTTGTCTTTAGCCATTCCATG |

In some embodiments, an influenza A region 1 second primer comprises a target hybridizing region 15-30 nucleobases in length, wherein the target hybridizing region is contained within SEQ ID NO:18 an RNA equivalent or a DNA/RNA chimeric thereof and contains the nucleotide sequence of SEQ ID NO:19 or SEQ ID NO:20 an RNA equivalent or a DNA/RNA chimeric thereof (Table 1b). In some embodiments, an influenza A region 1 second primer comprises a target hybridizing region 15-30 nucleobases in length, wherein the target hybridizing region is contained within SEQ ID NO:18 an RNA equivalent or a DNA/RNA chimeric thereof and contains a nucleotide sequence differing by no more than 0, 1, 2, 3, 4, or 5 nucleotides from the nucleotide sequence of SEQ ID NO:19 or SEQ ID NO:20 or an RNA equivalent or a DNA/RNA chimeric thereof. In some embodiments, the influenza A region 1 second primer comprises the nucleotide sequence of SEQ ID NO:38, 39, 40, 41, 42, or 43 or an RNA equivalent or a DNA/RNA chimeric thereof. In some embodiments, the influenza A region 1 second primer consists essentially of the nucleotide sequence of SEQ ID NO:38, 39, 40, 41, 42, or 43 or an RNA equivalent or a DNA/RNA chimeric thereof. In some embodiments, the influenza A region 1 second primer consists of the nucleotide sequence of SEQ ID NO:38, 39, 40, 41, 42, or 43 or an RNA equivalent or a DNA/RNA chimeric thereof. In some embodiments, the influenza A region 1 second primer comprises a nucleotide sequence having no more than 0, 1, 2, 3, 4, or 5 mismatches from the nucleotide sequence of SEQ ID NO:38, 39, 40, 41, 42, or 43 or an RNA equivalent or a DNA/RNA chimeric thereof. In some embodiments, the influenza A region 1 second primer comprises a nucleotide sequence having at least 80%, at least 85% at least 90%, or at least 95% homology to the nucleotide sequence of SEQ ID NO:38, 39, 40, 41, 42, or 43 or an RNA equivalent or a DNA/RNA chimeric thereof. An influenza A region 1 second primer can have 1 or more modified nucleotides or nucleotide analogs. In some embodiments, two different second influenza A region 1 primers are used in a TMA reaction.

In some embodiments, an influenza A region 1 second primer comprises an influenza A region 1 promoter primer. In some embodiments, the influenza A region 1 promoter primer comprises the nucleotide sequence of SEQ ID NO: 28, 29, 30, 31, 32, or 33 or an RNA equivalent or a DNA/RNA chimeric thereof (Table 1b). In some embodiments, the influenza A region 1 promoter primer consists essentially of the nucleotide sequence of SEQ ID NO: 28, 29, 30, 31, 32, or 33 or an RNA equivalent or a DNA/RNA chimeric thereof. In some embodiments, the influenza A region 1 promoter primer consists of the nucleotide sequence of SEQ ID NO: 28, 29, 30, 31, 32, or 33 or an RNA equivalent or a DNA/RNA chimeric thereof. In some embodiments, the influenza A region 1 promoter primer comprises a nucleotide sequence having no more than 0, 1, 2, 3, 4, or 5 mismatches from the nucleotide sequence of SEQ ID NO: 28, 29, 30, 31, 32, or 33 or an RNA equivalent or a DNA/RNA chimeric thereof. In some embodiments, the influenza A region 1 promoter primer comprises a nucleotide sequence having at least 80%, at least 85% at least 90%, or at least 95% homology to the nucleotide sequence of SEQ ID NO:38, 39, 40, 41, 42, or 43 or an RNA equivalent or a DNA/RNA chimeric thereof. An influenza A region 1 promoter primer can have 1 or more modified nucleotides or nucleotide analogs. In some embodiments, two different influenza A region 1 promoter primers are used in a TMA reaction.

TABLE 1b
Influenza A region 1 second/promoter primer sequences

| SEQ ID NO: | Sequence (5′→3′) |
|---|---|
| 18 | cgtcaggccccctcaaagccgagatcgcacaaagact |
| 19 | caggccccctcaaagccgag |
| 20 | caaagccgagatcg |
| 38 | ATCAGGCCCCCTCAAAGCCGAGATCGC |
| 28 | AATTTAATACGACTCACTATAGGGAGAATCAGGCCCCCTCAAAGCCGAGATCGC |
| 39 | CATCAGGCCCCCTCAAAGCCGAGATC |
| 29 | AATTTAATACGACTCACTATAGGGAGACATCAGGCCCCCTCAAAGCCGAGATC |
| 40 | CAGGCCCCCTCAAAGCCGAG |
| 30 | AATTTAATACGACTCACTATAGGGAGACAGGCCCCCTCAAAGCCGAG |
| 41 | CAGGCCCCCTCAAAGCCGAGATCG |
| 31 | AATTTAATACGACTCACTATAGGGAGACAGGCCCCCTCAAAGCCGAGATCG |
| 42 | CCCTCAAAGCCGAGATCGCGCAGAG |
| 32 | AATTTAATACGACTCACTATAGGGAGACCCTCAAAGCCGAGATCGCGCAGAG |
| 43 | CAAAGCCGAGATCGCGCAGAGACT |
| 33 | AATTTAATACGACTCACTATAGGGAGACAAAGCCGAGATCGCGCAGAGACT |

In some embodiments, an influenza A region 1 probe comprises a target hybridizing region 22-40 nucleobases in length, wherein the target hybridizing region is contained within SEQ ID NO:12 or SEQ ID NO: 13 or a DNA equivalent, DNA/RNA chimeric, and/or a complement thereof and contains the nucleotide sequence of SEQ ID NO:14 or a DNA equivalent, DNA/RNA chimeric, and/or a complement thereof (Table 1c). In some embodiments, an influenza A region 1 probe comprises a target hybridizing region 22-40 nucleobases in length, wherein the target hybridizing region is contained within SEQ ID NO:12 or SEQ ID NO: 13 or a DNA, DNA/RNA chimeric, and/or a complement thereof and contains a nucleotide sequence differing by no more than 0, 1, 2, 3, 4, or 5 nucleotides from the nucleotide sequence of SEQ ID NO:14 or a DNA, DNA/RNA chimeric, and/or a complement thereof. In some embodiments, the influenza A region 1 probe comprises the nucleotide sequence of SEQ ID NO:63, 64, 65, 66, 67, or 68 or a DNA equivalent a DNA/RNA chimeric, and/or a complement thereof. In some embodiments, the influenza A region 1 probe consists essentially of the nucleotide sequence of SEQ ID NO: 63, 64, 65, 66, 67, or 68 or a DNA equivalent, a DNA/RNA chimeric, and/or a complement thereof. In some embodiments, the influenza A region 1 probe consists of the nucleotide sequence of SEQ ID NO: 63, 64, 65, 66, 67, or 68 or a DNA equivalent, a DNA/RNA chimeric, and/or a complement thereof. In some embodiments, the influenza A region 1 probe comprises a nucleotide sequence having no more than 0, 1, 2, 3, 4, or 5 mismatches from the nucleotide sequence of SEQ ID NO: 63, 64, 65, 66, 67, or 68 or a DNA equivalent, a DNA/RNA chimeric, and/or a complement thereof. In some embodiments, the influenza A region 1 probe comprises a nucleotide sequence having at least 80%, at least 85% at least 90%, or at least 95% homology to the nucleotide sequence of SEQ ID NO: 63, 64, 65, 66, 67, or 68 or a DNA equivalent, a DNA/RNA chimeric, and/or a complement thereof. An influenza A region 1 probe can have 1 or more modified nucleotides or nucleotide analogs.

In some embodiments, an influenza A region 1 probe comprises an influenza A region 1 torch. In some embodiments, the influenza A region 1 torch comprises the nucleotide sequence of SEQ ID NO: 54, 55, 56, 57, 58, or 59 or a DNA equivalent, a DNA/RNA chimeric, and/or a complement thereof (Table 1c). In some embodiments, the influenza A region 1 torch consists essentially of the nucleotide sequence of SEQ ID NO: 54, 55, 56, 57, 58, or 59 or a DNA equivalent, a DNA/RNA chimeric, and/or a complement thereof. In some embodiments, the influenza A region 1 torch consists of the nucleotide sequence of SEQ ID NO: 54, 55, 56, 57, 58, or 59 or a DNA equivalent, a DNA/RNA chimeric, and/or a complement thereof. In some embodiments, the influenza A region 1 torch comprises a nucleotide sequence having no more than 0, 1, 2, 3, 4, or 5 mismatches from the nucleotide sequence of SEQ ID NO: 54, 55, 56, 57, 58, or 59 or a DNA equivalent, a DNA/RNA chimeric, and/or a complement thereof. In some embodiments, the influenza A region 1 torch comprises a nucleotide sequence having at least 80%, at least 85% at least 90%, or at least 95% homology to the nucleotide sequence of SEQ ID NO: 54, 55, 56, 57, 58, or 59 or a DNA equivalent, a DNA/RNA chimeric, and/or a complement thereof. An influenza A region 1 torch can have 1 or more modified nucleotides or nucleotide analogs.

TABLE 1c
Influenza A region 1 probe sequences

| SEQ ID NO: | Sequence (5′→3′) |
|---|---|
| 12 | ggncnngncnnnagccannccangagagccncaagan |
| | cngngnnynnyccwgcaaakacacnnnc (n = t |
| | or u) |
| 13 | ucaagaucuguguuyuuyccwgcaaakacacuuuc |
| 14 | cuguguuyuuyccwgcaaakac |
| 63 | GCAAGAUCUGUGUUCUUUCCUGCAAAGACA |
| 54 | GCAAGAUCUGUGUUCUUUCCUGCAAAGACAUCUUGC |
| 64 | GCAAGAUCUGUGUUUUUCCCAGCAAAGACA |
| 55 | GCAAGAUCUGUGUUUUUCCCAGCAAAGACAUCUUGC |
| 65 | GAUCUGUGUUUUUCCCAGCAAAGACAUCUUC |
| 56 | GAUCUGUGUUUUUCCCAGCAAAGACAUCUUCAGAUC |
| 66 | UCUGUGUUUUUCCCAGCAAAGACAUCU |
| 57 | UCUGUGUUUUUCCCAGCAAAGACAUCUACAGA |
| 67 | GAUCUGUGUUUUUCCCAGCAAAGACA |
| 58 | GAUCUGUGUUUUUCCCAGCAAAGACAAGAUC |
| 68 | CUGUGUUUUUCCCAGCAAAGAC |
| 59 | CUGUGUUUUUCCCAGCAAAGACCACAG |

An influenza A region 1 primer set includes at least one first primer and at least 1 second primer as described above. In some embodiments, an Influenza A region 1 primer set includes at least one first primer and at least 2 different second primers.

B. Influenza a Region 2

In some embodiments, an influenza A region 2 first primer comprises a target hybridizing region 15-30 nucleobases in length, wherein the target hybridizing region is contained within SEQ ID NO: 25 or an RNA equivalent or a DNA/RNA chimeric thereof and contains the nucleotide sequence of SEQ ID NO: 26 or SEQ ID NO: 27, or an RNA equivalent or a DNA/RNA chimeric thereof (Table 1d). In some embodiments, an influenza A region 2 first primer comprises a target hybridizing region 15-30 nucleobases in length, wherein the target hybridizing region is contained within SEQ ID NO: 25 an RNA equivalent or a DNA/RNA chimeric thereof and contains a nucleotide sequence differing by no more than 0, 1, 2, 3, 4, or 5 nucleotides from the nucleotide sequence of SEQ ID NO: 26 or SEQ ID NO: 27 or an RNA equivalent or a DNA/RNA chimeric thereof. In some embodiments, the influenza A region 2 first primer comprises the nucleotide sequence of SEQ ID NO: 6, 7, 8, 9, 10, or 11 or an RNA equivalent or a DNA/RNA chimeric thereof. In some embodiments, the influenza A region 2 first primer consists essentially of the nucleotide sequence of SEQ ID NO: 6, 7, 8, 9, 10, or 11 or an RNA equivalent or a DNA/RNA chimeric thereof. In some embodiments, the influenza A region 2 first primer consists of the nucleotide sequence of SEQ ID NO: 6, 7, 8, 9, or 11 or an RNA equivalent or a DNA/RNA chimeric thereof. In some embodiments, the influenza A region 2 first primer comprises a nucleotide sequence having no more than 0, 1, 2, 3, 4, or 5 mismatches from the nucleotide sequence of SEQ ID NO: 6, 7, 8, 9, 10, or 11 or an RNA equivalent or a DNA/RNA chimeric thereof. In some embodiments, the influenza A region 2 first primer comprises a nucleotide sequence having at least 80%, at least 85% at least 90%, or at least 95% homology to the nucleotide sequence of SEQ ID NO: 6, 7, 8, 9, 10, or 11 or an RNA equivalent or a DNA/RNA chimeric thereof. An influenza A region 2 first primer can have 1 or more modified nucleotides or nucleotide analogs. In some embodiments, the influenza A region 2 first primer is an NT7 primer.

TABLE 1d
Influenza A region 2 NT7 primer sequences

| SEQ ID NO: | Sequence (5′→3′) |
|---|---|
| 25 | gggtcyccattyccattkagggcattttggataaagc |
| 26 | cattkagggcattttgga |
| 27 | cattyccattkagggc |
| 6 | CCCATTGAGGGCATTTTGGACAAAGC |
| 7 | CATTGAGGGCATTTTGGACAAAG |
| 8 | CCATTGAGGGCATTTTGGACAA |
| 9 | CATTCCCATTGAGGGCATTTTGGACA |
| 10 | CTCCATTCCCATTGAGGGCATTTTGGA |
| 11 | GGGTCTCCATTCCCATTGAGGGC |

In some embodiments, an influenza A region 2 second primer comprises a target hybridizing region 15-30 nucleobases in length, wherein the target hybridizing region is contained within SEQ ID NO:21 or an RNA equivalent or a DNA/RNA chimeric thereof and contains the nucleotide sequence of SEQ ID NO: 22 or an RNA equivalent or a DNA/RNA chimeric thereof (Table 1e). In some embodiments, an influenza A region 2 second primer comprises a target hybridizing region 15-30 nucleobases in length, wherein the target hybridizing region is contained within SEQ ID NO: 21 or an RNA equivalent or a DNA/RNA chimeric thereof and contains a nucleotide sequence differing by no more than 0, 1, 2, 3, 4, or nucleotides from the nucleotide sequence of SEQ ID NO:22 or an RNA equivalent or a DNA/RNA chimeric thereof. In some embodiments, the influenza A region 2 second primer comprises the nucleotide sequence of SEQ ID NO: 44, 45, 46, or 47 or an RNA equivalent or a DNA/RNA chimeric thereof. In some embodiments, the influenza A region 2 second primer consists essentially of the nucleotide sequence of SEQ ID NO: 44, 45, 46, or 47 or an RNA equivalent or a DNA/RNA chimeric thereof. In some embodiments, the influenza A region 2 second primer consists of the nucleotide sequence of SEQ ID NO: 44, 45, 46, or 47 or an RNA equivalent or a DNA/RNA chimeric thereof. In some embodiments, the influenza A region 2 second primer comprises a nucleotide sequence having no more than 0, 1, 2, 3, 4, or 5 mismatches from the nucleotide sequence of SEQ ID NO: 44, 45, 46, or 47 or an RNA equivalent or a DNA/RNA chimeric thereof. In some embodiments, the influenza A region 2 second primer comprises a nucleotide sequence having at least 80%, at least 85% at least 90%, or at least 95% homology to the nucleotide sequence of SEQ ID NO: 44, 45, 46, or 47 or an RNA equivalent or a DNA/RNA chimeric thereof. An influenza A region 2 second primer can have 1 or more modified nucleotides or nucleotide analogs. In some embodiments, two different second influenza A region 2 primers are used in a TMA reaction.

In some embodiments, an influenza A region 2 second primer comprises an influenza A region 2 promoter primer. In some embodiments, the influenza A region 2 promoter primer comprises the nucleotide sequence of SEQ ID NO: 34, 35, 36, or 37 or an RNA equivalent or a DNA/RNA chimeric thereof (Table 1e). In some embodiments, the influenza A region 2 promoter primer consists essentially of the nucleotide sequence of SEQ ID NO: 34, 35, 36, or 37 or an RNA equivalent or a DNA/RNA chimeric thereof. In some embodiments, the influenza A region 2 promoter primer consists of the nucleotide sequence of SEQ ID NO: 34, 36, or 37 or an RNA equivalent or a DNA/RNA chimeric thereof. In some embodiments, the influenza A region 2 promoter primer comprises a nucleotide sequence having no more than 0, 1, 2, 3, 4, or 5 mismatches from the nucleotide sequence of SEQ ID NO: 34, 35, 36, or 37 or an RNA equivalent or a DNA/RNA chimeric thereof. In some embodiments, the influenza A region 2 promoter primer comprises a nucleotide sequence having at least 80%, at least 85% at least 90%, or at least 95% homology to the nucleotide sequence of SEQ ID NO: 34, 35, 36, or 37 or an RNA equivalent or a DNA/RNA chimeric thereof. An influenza A region 2 promoter primer can have 1 or more modified nucleotides or nucleotide analogs. In some embodiments, two different influenza A region 1 promoter primers are used in a TMA reaction.

TABLE 1e
Influenza A region 2 second/promoter primer sequences

| SEQ ID NO: | Sequence (5′→3′) |
|---|---|
| 21 | gratttgtgttcacgctcaccgtgcccagtgagcg |
| 22 | gtgttcacgctcaccgtgcccag |
| 44 | GGGTTTGTGTTCACGCTCACCGTGCCCAGTG |
| 34 | AATTTAATACGACTCACTATAGGGAGAGGGTTTGTGTTCACGCTCACCGTGCCCAG |
| | TG |
| 45 | GTTTGTGTTCACGCTCACCGTGCCCAG |
| 35 | AATTTAATACGACTCACTATAGGGAGAGTTTGTGTTCACGCTCACCGTGCCCAG |
| 46 | GTTTGTGTTCACGCTCACCGTGCCCAGTGA |
| 36 | AATTTAATACGACTCACTATAGGGAGAGTTTGTGTTCACGCTCACCGTGCCCAGTG |
| | A |
| 47 | GTGTTCACGCTCACCGTGCCCAGTGAGCG |
| 37 | AATTTAATACGACTCACTATAGGGAGAGTGTTCACGCTCACCGTGCCCAGTGAGCG |

In some embodiments, an influenza A region 2 probe comprises a target hybridizing region 19-25 nucleobases in length, wherein the target hybridizing region is contained within SEQ ID NO: 23 or a DNA equivalent, a DNA/RNA chimeric, and/or a complement thereof and contains the nucleotide sequence of SEQ ID NO: 24 or a DNA equivalent, a DNA/RNA chimeric, and/or a complement thereof (Table 1f). In some embodiments, an influenza A region 2 probe comprises a target hybridizing region 19-25 nucleobases in length, wherein the target hybridizing region is contained within SEQ ID NO: 23 or a DNA equivalent, a DNA/RNA chimeric, and/or a complement thereof and contains a nucleotide sequence differing by no more than 0, 1, 2, 3, 4, or 5 nucleotides from the nucleotide sequence of SEQ ID NO: 24 or a DNA equivalent, a DNA/RNA chimeric, and/or a complement thereof. In some embodiments, the influenza A region 2 probe comprises the nucleotide sequence of SEQ ID NO: 69, 70, or 71 or a DNA equivalent, a DNA/RNA chimeric, and/or a complement thereof. In some embodiments, the influenza A region 2 probe consists essentially of the nucleotide sequence of SEQ ID NO: 69, 70, or 71 or a DNA equivalent, a DNA/RNA chimeric, and/or a complement thereof. In some embodiments, the influenza A region 2 probe consists of the nucleotide sequence of SEQ ID NO: 69, 70, or 71 or a DNA equivalent, a DNA/RNA chimeric, and/or a complement thereof. In some embodiments, the influenza A region 2 probe comprises a nucleotide sequence having no more than 0, 1, 2, 3, 4, or 5 mismatches from the nucleotide sequence of SEQ ID NO: 69, 70, or 71 a DNA equivalent, a DNA/RNA chimeric, and/or a complement thereof. In some embodiments, the influenza A region 2 probe comprises a nucleotide sequence having at least 80%, at least 85% at least 90%, or at least 95% homology to the nucleotide sequence of SEQ ID NO: 69, 70, or 71 or a DNA equivalent, a DNA/RNA chimeric, and/or a complement thereof. An influenza A region 2 probe can have 1 or more modified nucleotides or nucleotide analogs.

In some embodiments, an influenza A region 2 probe comprises an influenza A region 2 torch. In some embodiments, the influenza A region 2 torch comprises the nucleotide sequence of SEQ ID NO: 60, 61, or 62 or a DNA equivalent, a DNA/RNA chimeric, and/or a complement thereof (Table 1f). In some embodiments, the influenza A region 2 torch consists essentially of the nucleotide sequence of SEQ ID NO: 60, 61, or 62 or a DNA equivalent, a DNA/RNA chimeric, and/or a complement thereof. In some embodiments, the influenza A region 2 torch consists of the nucleotide sequence of SEQ ID NO: 60, 61, or 62 or a DNA equivalent, a DNA/RNA chimeric, and/or a complement thereof. In some embodiments, the influenza A region 2 torch comprises a nucleotide sequence having no more than 0, 1, 2, 3, 4, or 5 mismatches from the nucleotide sequence of SEQ ID NO: 60, 61, or 62 or a DNA equivalent, a DNA/RNA chimeric, and/or a complement thereof. In some embodiments, the influenza A region 2 torch comprises a nucleotide sequence having at least 80%, at least 85% at least 90%, or at least 95% homology to the nucleotide sequence of SEQ ID NO: 60, 61, or 62 or a DNA equivalent, a DNA/RNA chimeric, and/or a complement thereof. An influenza A region 2 torch can have 1 or more modified nucleotides or nucleotide analogs.

TABLE 1f
Influenza A region 2 probe sequences

| SEQ ID NO: | Sequence (5′→3′) |
|---|---|
| 23 | agcgucuacgcugcaguccucgcuc |
| 24 | cgucuacgcugcaguccuc |
| 69 | CGUCUACGCUGCAGUCCUCGCUC |
| 60 | CGUCUACGCUGCAGUCCUCGCUCGACG |
| 70 | GCGUCUACGCUGCAGUCCUCG |
| 61 | GCGUCUACGCUGCAGUCCUCGACGC |
| 71 | AGCGUCUACGCUGCAGUCCUC |
| 62 | AGCGUCUACGCUGCAGUCCUCCGCU |

An influenza A region 2 primer set includes at least one first primer and at least 1 second primer as described above. In some embodiments, an Influenza A region 2 primer set includes at least one first primer and at least 2 different second primers.

C. Influenza B Region 1

In some embodiments, an influenza B region 1 first primer comprises a target hybridizing region 20-30 nucleobases in length, wherein the target hybridizing region is contained within SEQ ID NO: 90 or an RNA equivalent or a DNA/RNA chimeric thereof and contains the nucleotide sequence of SEQ ID NO: 91 or an RNA equivalent or a DNA/RNA chimeric thereof (Table 2a). In some embodiments, an influenza B region 1 first primer comprises a target hybridizing region 20-30 nucleobases in length, wherein the target hybridizing region is contained within SEQ ID NO: 90 or an RNA equivalent or a DNA/RNA chimeric thereof and contains a nucleotide sequence differing by no more than 0, 1, 2, 3, 4, or nucleotides from the nucleotide sequence of SEQ ID NO: 91 or an RNA equivalent or a DNA/RNA chimeric thereof. In some embodiments, the influenza B region 1 first primer comprises the nucleotide sequence of SEQ ID NO: 72, 73, 74, or 75 or an RNA equivalent or a DNA/RNA chimeric thereof. In some embodiments, the influenza B region 1 first primer consists essentially of the nucleotide sequence of SEQ ID NO: 72, 73, 74, or 75 or an RNA equivalent or a DNA/RNA chimeric thereof. In some embodiments, the influenza B region 1 first primer consists of the nucleotide sequence of SEQ ID NO: 72, 73, 74, or 75 or an RNA equivalent or a DNA/RNA chimeric thereof. In some embodiments, the influenza B region 1 first primer comprises a nucleotide sequence having no more than 0, 1, 2.3, 4, or 5 mismatches from the nucleotide sequence of SEQ ID NO: 72, 73, 74, or 75 or an RNA equivalent or a DNA/RNA chimeric thereof. In some embodiments, the influenza B region 1 first primer comprises a nucleotide sequence having at least 80%, at least 85% at least 90%, or at least 95% homology to the nucleotide sequence of SEQ ID NO: 72, 73, 74, or 75 or an RNA equivalent or a DNA/RNA chimeric thereof. An influenza B region 1 first primer can have 1 or more modified nucleotides or nucleotide analogs. In some embodiments, the influenza B region 1 first primer is an NT7 primer.

TABLE 2a
Influenza B region 1 NT7 primer sequences

| SEQ ID NO: | Sequence (5′→3′) |
|---|---|
| 90 | cattccaaggcagagtctaggtcaaattctttyc |
| 91 | gcagagtctaggtcaaattc |
| 72 | GCAGAGTCTAGGTCAAATTCTTTCC |
| 73 | GCAGAGTCTAGGTCAAATTCTTTTC |
| 74 | CAAGGCAGAGTCTAGGTCAAATTCTTTC |
| 75 | CATTCCAAGGCAGAGTCTAGGTCAAATTC |

In some embodiments, an influenza B region 1 second primer comprises a target hybridizing region 17-30 nucleobases in length, wherein the target hybridizing region is contained within SEQ ID NO: 85 or an RNA equivalent or a DNA/RNA chimeric thereof and contains the nucleotide sequence of SEQ ID NO: 86 or SEQ ID NO: 87 or an RNA equivalent or a DNA/RNA chimeric thereof (Table 2b). In some embodiments, an influenza B region 1 second primer comprises a target hybridizing region 17-30 nucleobases in length, wherein the target hybridizing region is contained within SEQ ID NO: 85 or an RNA equivalent or a DNA/RNA chimeric thereof and contains a nucleotide sequence differing by no more than 0, 1, 2, 3, 4, or 5 nucleotides from the nucleotide sequence of SEQ ID NO: 86 or SEQ ID NO: 87 or an RNA equivalent or a DNA/RNA chimeric thereof. In some embodiments, the influenza B region 1 second primer comprises the nucleotide sequence of SEQ ID NO: 104, 105, 106, or 107 or an RNA equivalent or a DNA/RNA chimeric thereof. In some embodiments, the influenza B region 1 second primer consists essentially of the nucleotide sequence of SEQ ID NO: 104, 105, 106, or 107 or an RNA equivalent or a DNA/RNA chimeric thereof. In some embodiments, the influenza B region 1 second primer consists of the nucleotide sequence of SEQ ID NO: 104, 105, 106, or 107 or an RNA equivalent or a DNA/RNA chimeric thereof. In some embodiments, the influenza B region 1 second primer comprises a nucleotide sequence having no more than 0, 1, 2, 3, 4, or 5 mismatches from the nucleotide sequence of SEQ ID NO: 104, 105, 106, or 107 or an RNA equivalent or a DNA/RNA chimeric thereof. In some embodiments, the influenza B region 1 second primer comprises a nucleotide sequence having at least 80%, at least 85% at least 90%, or at least 95% homology to the nucleotide sequence of SEQ ID NO: 104, 105, 106, or 107 or an RNA equivalent or a DNA/RNA chimeric thereof. An influenza B region 1 second primer can have 1 or more modified nucleotides or nucleotide analogs. In some embodiments, two different second influenza B region 1 primers are used in a TMA reaction.

In some embodiments, an influenza B region 1 second primer comprises an influenza B region 1 promoter primer. In some embodiments, the influenza B region 1 promoter primer comprises the nucleotide sequence of SEQ ID NO: 96, 97, 98, or 99 or an RNA equivalent or a DNA/RNA chimeric thereof (Table 2b). In some embodiments, the influenza B region 1 promoter primer consists essentially of the nucleotide sequence of SEQ ID NO: 96, 97, 98, or 99 or an RNA equivalent or a DNA/RNA chimeric thereof. In some embodiments, the influenza B region 1 promoter primer consists of the nucleotide sequence of SEQ ID NO: 96, 97, 98, or 99 or an RNA equivalent or a DNA/RNA chimeric thereof. In some embodiments, the influenza B region 1 promoter primer comprises a nucleotide sequence having no more than 0, 1, 2, 3, 4, or 5 mismatches from the nucleotide sequence of SEQ ID NO: 96, 97, 98, or 99 or an RNA equivalent or a DNA/RNA chimeric thereof. In some embodiments, the influenza B region 1 promoter primer comprises a nucleotide sequence having at least 80%, at least 85% at least 90%, or at least 95% homology to the nucleotide sequence of SEQ ID NO: 96, 97, 98, or 99 or an RNA equivalent or a DNA/RNA chimeric thereof. An influenza B region 1 promoter primer can have 1 or more modified nucleotides or nucleotide analogs. In some embodiments, two different influenza B region 2 promoter primers are used in a TMA reaction.

TABLE 2b
Influenza B region 1 second/promoter primer sequences

| SEQ ID NO: | Sequence (5′→3′) |
|---|---|
| 85 | cctgctttcattgacagaagatggagaaggcaaagcag |
| 86 | cattgacagaagatggagaag |
| 87 | cagaagatggagaaggc |
| 104 | CCTGCTTTCATTGACAGAAGATGGAGAAG |
| 96 | AATTTAATACGACTCACTATAGGGAGACCTGCTTTCATTGACAGAAGATGGAGAAG |
| 105 | CTTTCATTGACAGAAGATGGAGAAGGC |
| 97 | AATTTAATACGACTCACTATAGGGAGACTTTCATTGACAGAAGATGGAGAAGGC |
| 106 | CATTGACAGAAGATGGAGAAGGCAAAGC |
| 98 | AATTTAATACGACTCACTATAGGGAGACATTGACAGAAGATGGAGAAGGCAAAGC |
| 107 | CAGAAGATGGAGAAGGCAAAGCAG |
| 99 | AATTTAATACGACTCACTATAGGGAGACAGAAGATGGAGAAGGCAAAGCAG |

In some embodiments, an influenza B region 1 probe comprises a target hybridizing region 23-35 nucleobases in length, wherein the target hybridizing region is contained within SEQ ID NO: 88 or a DNA equivalent, a DNA/RNA chimeric, and/or a complement thereof and contains the nucleotide sequence of SEQ ID NO: 89 or a DNA equivalent, a DNA/RNA chimeric, and/or a complement thereof (Table 2c). In some embodiments, an influenza B region 1 probe comprises a target hybridizing region 23-35 nucleobases in length, wherein the target hybridizing region is contained within SEQ ID NO: 88 or a DNA equivalent, a DNA/RNA chimeric, and/or a complement thereof and contains a nucleotide sequence differing by no more than 0, 1, 2, 3, 4, or 5 nucleotides from the nucleotide sequence of SEQ ID NO: 89 or a DNA equivalent, a DNA/RNA chimeric, and/or a complement thereof. In some embodiments, the influenza B region 1 probe comprises the nucleotide sequence of SEQ ID NO: 120, 121, or 122 or a DNA equivalent, a DNA/RNA chimeric, and/or a complement thereof. In some embodiments, the influenza B region 1 probe consists essentially of the nucleotide sequence of SEQ ID NO: 120, 121, or 122 or a DNA equivalent, a DNA/RNA chimeric, and/or a complement thereof. In some embodiments, the influenza B region 1 probe consists of the nucleotide sequence of SEQ ID NO: 120, 121, or 122 or a DNA equivalent, a DNA/RNA chimeric, and/or a complement thereof. In some embodiments, the influenza B region 1 probe comprises a nucleotide sequence having no more than 0, 1, 2, 3, 4, or 5 mismatches from the nucleotide sequence of SEQ ID NO: 120, 121, or 122 or a DNA equivalent, a DNA/RNA chimeric, and/or a complement thereof. In some embodiments, the influenza B region 1 probe comprises a nucleotide sequence having at least 80%, at least 85% at least 90%, or at least 95% homology to the nucleotide sequence of SEQ ID NO: 120, 121, or 122 or a DNA equivalent, a DNA/RNA chimeric, and/or a complement thereof. An influenza B region 1 probe can have 1 or more modified nucleotides or nucleotide analogs.

In some embodiments, an influenza B region 1 probe comprises an influenza B region 1 torch. In some embodiments, the influenza B region 1 torch comprises the nucleotide sequence of SEQ ID NO: 114, 115, or 116 or a DNA equivalent, a DNA/RNA chimeric, and/or a complement thereof (Table 2c). In some embodiments, the influenza B region 1 torch consists essentially of the nucleotide sequence of SEQ ID NO: 114, 115, or 116 or a DNA equivalent, a DNA/RNA chimeric, and/or a complement thereof. In some embodiments, the influenza B region 1 torch consists of the nucleotide sequence of SEQ ID NO: 114, 115, or 116 or a DNA equivalent, a DNA/RNA chimeric, and/or a complement thereof. In some embodiments, the influenza B region 1 torch comprises a nucleotide sequence having no more than 0, 1, 2, 3, 4, or 5 mismatches from the nucleotide sequence of SEQ ID NO: 114, 115, or 116 or a DNA equivalent, a DNA/RNA chimeric, and/or a complement thereof. In some embodiments, the influenza B region 1 torch comprises a nucleotide sequence having at least 80%, at least 85% at least 90%, or at least 95% homology to the nucleotide sequence of SEQ ID NO: 114, 115, or 116 or a DNA equivalent, a DNA/RNA chimeric, and/or a complement thereof. An influenza B region 1 torch can have 1 or more modified nucleotides or nucleotide analogs.

TABLE 2c
Influenza B region 1 probe sequences

| SEQ ID NO: | Sequence (5′→3′) |
|---|---|
| 88 | caccgaaccaacaguguaauuuuucugcuag |
| 89 | gaaccaacaguguaauuuuucug |
| 120 | CGAACCAACAGUGUAAUUUUUCUGCUAG |
| 114 | CGAACCAACAGUGUAAUUUUUCUGCUAGGUUCG |
| 121 | GAACCAACAGUGUAAUUUUUCUGC |
| 115 | GAACCAACAGUGUAAUUUUUCUGCGGUUC |
| 122 | CACCGAACCAACAGUGUAAUUUUUCUGC |
| 116 | CACCGAACCAACAGUGUAAUUUUUCUGCCGGUG |

An influenza B region 1 primer set includes at least one first primer and at least 1 second primer as described above. In some embodiments, an Influenza B region 1 primer set includes at least one first primer and at least 2 different second primers.

D. Influenza B Region 2

In some embodiments, an influenza B region 2 first primer comprises a target hybridizing region 13-30 nucleobases in length, wherein the target hybridizing region is contained within SEQ ID NO: 83 or an RNA equivalent or a DNA/RNA chimeric thereof and contains the nucleotide sequence of SEQ ID NO: 84 or an RNA equivalent or a DNA/RNA chimeric thereof (Table 2d). In some embodiments, an influenza B region 2 first primer comprises a target hybridizing region 13-30 nucleobases in length, wherein the target hybridizing region is contained within SEQ ID NO: 83 or an RNA equivalent or a DNA/RNA chimeric thereof and contains a nucleotide sequence differing by no more than 0, 1, 2, 3, 4, or nucleotides from the nucleotide sequence of SEQ ID NO: 84 or an RNA equivalent or a DNA/RNA chimeric thereof. In some embodiments, the influenza B region 2 first primer comprises the nucleotide sequence of SEQ ID NO: 77 or 78, or an RNA equivalent or a DNA/RNA chimeric thereof. In some embodiments, the influenza B region 2 first primer consists essentially of the nucleotide sequence of SEQ ID NO: 77 or 78 or an RNA equivalent or a DNA/RNA chimeric thereof. In some embodiments, the influenza B region 2 first primer consists of the nucleotide sequence of SEQ ID NO: 77 or 78 or an RNA equivalent or a DNA/RNA chimeric thereof. In some embodiments, the influenza B region 2 first primer comprises a nucleotide sequence having no more than 0, 1, 2, 3, 4, or 5 mismatches from the nucleotide sequence of SEQ ID NO: 77 or 78 or an RNA equivalent or a DNA/RNA chimeric thereof. In some embodiments, the influenza B region 2 first primer comprises a nucleotide sequence having at least 80%, at least 85% at least 90%, or at least 95% homology to the nucleotide sequence of SEQ ID NO: 77 or 78 or an RNA equivalent or a DNA/RNA chimeric thereof. An influenza B region 2 first primer can have 1 or more modified nucleotides or nucleotide analogs. In some embodiments, the influenza B region 2 first primer is an NT7 primer.

TABLE 2d
Influenza B region 2 NT7 primer sequences

| SEQ ID NO: | Sequence (5′→3′) |
|---|---|
| 83 | ctggtgatagtcggtgctcttgaccaaatt |
| 84 | cggtgctcttgac |
| 77 | CGGTGCTCTTGACCAAATT |
| 78 | CTGGTGATAATCGGTGCTCTTGAC |

In some embodiments, an influenza B region 2 second primer comprises a target hybridizing region 16-30 nucleobases in length, wherein the target hybridizing region is contained within SEQ ID NO: 79 or an RNA equivalent or a DNA/RNA chimeric thereof and contains the nucleotide sequence of SEQ ID NO: 80 or an RNA equivalent or a DNA/RNA chimeric thereof (Table 2e). In some embodiments, an influenza B region 2 second primer comprises a target hybridizing region 17-30 nucleobases in length, wherein the target hybridizing region is contained within SEQ ID NO: 79 or an RNA equivalent or a DNA/RNA chimeric thereof and contains a nucleotide sequence differing by no more than 0, 1, 2, 3, 4, or nucleotides from the nucleotide sequence of SEQ ID NO: 80 or an RNA equivalent or a DNA/RNA chimeric thereof. In some embodiments, the influenza B region 2 second primer comprises the nucleotide sequence of SEQ ID NO: 104, 105, 106, or 107 or an RNA equivalent or a DNA/RNA chimeric thereof. In some embodiments, the influenza B region 2 second primer consists essentially of the nucleotide sequence of SEQ ID NO: 100, 101, 102, or 103 or an RNA equivalent or a DNA/RNA chimeric thereof. In some embodiments, the influenza B region 2 second primer consists of the nucleotide sequence of SEQ ID NO: 100, 101, 102, or 103 or an RNA equivalent or a DNA/RNA chimeric thereof. In some embodiments, the influenza B region 2 second primer comprises a nucleotide sequence having no more than 0, 1, 2, 3, 4, or 5 mismatches from the nucleotide sequence of SEQ ID NO: 100, 101, 102, or 103 or an RNA equivalent or a DNA/RNA chimeric thereof. In some embodiments, the influenza B region 2 second primer comprises a nucleotide sequence having at least 80%, at least 85% at least 90%, or at least 95% homology to the nucleotide sequence of SEQ ID NO: 100, 101, 102, or 103 or an RNA equivalent or a DNA/RNA chimeric thereof. An influenza B region 2 second primer can have 1 or more modified nucleotides or nucleotide analogs. In some embodiments, two different second influenza B region 2 primers are used in a TMA reaction.

In some embodiments, an influenza B region 2 second primer comprises an influenza B region 2 promoter primer. In some embodiments, the influenza B region 2 promoter primer comprises the nucleotide sequence of SEQ ID NO: 92, 93, 94, or 95 or an RNA equivalent or a DNA/RNA chimeric thereof (Table 2e). In some embodiments, the influenza B region 2 promoter primer consists essentially of the nucleotide sequence of SEQ ID NO: 92, 93, 94, or or an RNA equivalent or a DNA/RNA chimeric thereof. In some embodiments, the influenza B region 2 promoter primer consists of the nucleotide sequence of SEQ ID NO: 92, 93, 94, or 95 or an RNA equivalent or a DNA/RNA chimeric thereof. In some embodiments, the influenza B region 2 promoter primer comprises a nucleotide sequence having no more than 0, 1, 2, 3, 4, or 5 mismatches from the nucleotide sequence of SEQ ID NO: 92, 93, 94, or or an RNA equivalent or a DNA/RNA chimeric thereof. In some embodiments, the influenza B region 2 promoter primer comprises a nucleotide sequence having at least 80%, at least 85% at least 90%, or at least 95% homology to the nucleotide sequence of SEQ ID NO: 92, 93, 94, or 95 or an RNA equivalent or a DNA/RNA chimeric thereof. An influenza B region 2 promoter primer can have 1 or more modified nucleotides or nucleotide analogs. In some embodiments, two different influenza B region 2 promoter primers are used in a TMA reaction.

TABLE 2e
Influenza B region 2 second/promoter primer sequences

| SEQ ID NO: | Sequence (5′→3′) |
|---|---|
| 79 | gaaggacattcaaagccaattcgagcagctgaaac |
| 80 | caaagccaattcgagc |
| 100 | GAAGGACATTCAAAGCCAATTCGAGC |
| 92 | AATTTAATACGACTCACTATAGGGAGAGAAGGACATTCAAAGCCAATTCGAGC |
| 101 | GGACATTCAAAGCCAATTCGAGCAG |
| 93 | AATTTAATACGACTCACTATAGGGAGAGGACATTCAAAGCCAATTCGAGCAG |
| 102 | CATTCAAAGCCAATTCGAGCAGCTG |
| 94 | AATTTAATACGACTCACTATAGGGAGACATTCAAAGCCAATTCGAGCAGCTG |
| 103 | CAAAGCCAATTCGAGCAGCTGAAAC |
| 95 | AATTTAATACGACTCACTATAGGGAGACAAAGCCAATTCGAGCAGCTGAAAC |

In some embodiments, an influenza B region 2 probe comprises a target hybridizing region 17-30 nucleobases in length, wherein the target hybridizing region is contained within SEQ ID NO: 81 or a DNA equivalent, a DNA/RNA chimeric, and/or a complement thereof and contains the nucleotide sequence of SEQ ID NO: 82 or a DNA equivalent, a DNA/RNA chimeric, and/or a complement thereof (Table 20. In some embodiments, an influenza B region 2 probe comprises a target hybridizing region 17-30 nucleobases in length, wherein the target hybridizing region is contained within SEQ ID NO: 81 or a DNA equivalent, a DNA/RNA chimeric, and/or a complement thereof and contains a nucleotide sequence differing by no more than 0, 1, 2, 3, 4, or 5 nucleotides from the nucleotide sequence of SEQ ID NO: 82 or a DNA equivalent, a DNA/RNA chimeric, and/or a complement thereof. In some embodiments, the influenza B region 2 probe comprises the nucleotide sequence of SEQ ID NO: 123, 124, or 125 or a DNA equivalent, a DNA/RNA chimeric, and/or a complement thereof. In some embodiments, the influenza B region 2 probe consists essentially of the nucleotide sequence of SEQ ID NO: 123, 124, or 125 or a DNA equivalent, a DNA/RNA chimeric, and/or a complement thereof. In some embodiments, the influenza B region 2 probe consists of the nucleotide sequence of SEQ ID NO: 123, 124, or 125 or a DNA equivalent, a DNA/RNA chimeric, and/or a complement thereof. In some embodiments, the influenza B region 2 probe comprises a nucleotide sequence having no more than 0, 1, 2, 3, 4, or 5 mismatches from the nucleotide sequence of SEQ ID NO: 123, 124, or 125 or a DNA equivalent, a DNA/RNA chimeric, and/or a complement thereof. In some embodiments, the influenza B region 2 probe comprises a nucleotide sequence having at least 80%, at least 85% at least 90%, or at least 95% homology to the nucleotide sequence of SEQ ID NO: 123, 124, or 125 or a DNA equivalent, a DNA/RNA chimeric, and/or a complement thereof. An influenza B region 2 probe can have 1 or more modified nucleotides or nucleotide analogs.

In some embodiments, an influenza B region 2 probe comprises an influenza B region 2 torch. In some embodiments, the influenza B region 2 torch comprises the nucleotide sequence of SEQ ID NO: 117, 118, or 119 or a DNA equivalent, a DNA/RNA chimeric, and/or a complement thereof (Table 20. In some embodiments, the influenza B region 2 torch consists essentially of the nucleotide sequence of SEQ ID NO: 117, 118, or 119 or a DNA equivalent, a DNA/RNA chimeric, and/or a complement thereof. In some embodiments, the influenza B region 2 torch consists of the nucleotide sequence of SEQ ID NO: 117, 118, or 119 or a DNA equivalent, a DNA/RNA chimeric, and/or a complement thereof. In some embodiments, the influenza B region 2 torch comprises a nucleotide sequence having no more than 0, 1, 2, 3, 4, or 5 mismatches from the nucleotide sequence of SEQ ID NO: 117, 118, or 119 or a DNA equivalent, a DNA/RNA chimeric, and/or a complement thereof. In some embodiments, the influenza B region 2 torch comprises a nucleotide sequence having at least 80%, at least 85% at least 90%, or at least 95% homology to the nucleotide sequence of SEQ ID NO: 117, 118, or 119 or a DNA equivalent, a DNA/RNA chimeric, and/or a complement thereof. An influenza B region 2 torch can have 1 or more modified nucleotides or nucleotide analogs.

TABLE 2f
Influenza B region 2 probe sequences

| SEQ ID NO: | Sequence (5′→3′) |
|---|---|
| 81 | caaauugsgauaagacucccaccgcaguuuca |
| 82 | sgauaagacucccaccg |
| 123 | CGAUAAGACUCCCACCGCAGUUUC |
| 117 | CGAUAAGACUCCCACCGCAGUUUCUAUCG |
| 124 | GGAUAAGACUCCCACCGCAG |
| 118 | GGAUAAGACUCCCACCGCAGUAUCC |
| 125 | CAUUGGGAUAAGACUCCCACCG |
| 119 | CAUUGGGAUAAGACUCCCACCGCAAUG |

An influenza B region 2 primer set includes at least one first primer and at least 1 second primer as described above. In some embodiments, an Influenza B region 2 primer set includes at least one first primer and at least 2 different second primers.

E. SARS-CoV-2 Region 1

In some embodiments, the SARS-CoV-2 region 1 first primer comprises the nucleotide sequence of SEQ ID NO: 126, or an RNA equivalent or a DNA/RNA chimeric thereof. In some embodiments, the SARS-CoV-2 region 1 first primer consists essentially of the nucleotide sequence of SEQ ID NO: 126 or an RNA equivalent or a DNA/RNA chimeric thereof (Table 3a). In some embodiments, the SARS-CoV-2 region 1 first primer consists of the nucleotide sequence of SEQ ID NO: 126 or an RNA equivalent or a DNA/RNA chimeric thereof. In some embodiments, the SARS-CoV-2 region 1 first primer comprises a nucleotide sequence having no more than 0, 1, 2, 3, 4, or 5 mismatches from the nucleotide sequence of SEQ ID NO: 126 or an RNA equivalent or a DNA/RNA chimeric thereof. In some embodiments, the SARS-CoV-2 region 1 first primer comprises a nucleotide sequence having at least 80%, at least 85% at least 90%, or at least 95% homology to the nucleotide sequence of SEQ ID NO: 126 or an RNA equivalent or a DNA/RNA chimeric thereof. A SARS-CoV-2 region 1 first primer can have 1 or more modified nucleotides or nucleotide analogs. In some embodiments, the SARS-CoV-2 region 1 first primer is an NT7 primer.

TABLE 3a
SARS-CoV-2 region 1 NT7 primer sequences

| SEQ ID NO: | Sequence (5′→3′) |
|---|---|
| 126 | GCTTGATGGCTTTATGGGTAGAATT |

In some embodiments, a SARS-CoV-2 region 1 second primer comprises a target hybridizing region 18-35 nucleobases in length, wherein the target hybridizing region is contained within SEQ ID NO: 140 or an RNA equivalent or a DNA/RNA chimeric thereof and contains the nucleotide sequence of SEQ ID NO: 141 or an RNA equivalent or a DNA/RNA chimeric thereof (Table 3b). In some embodiments, a SARS-CoV-2 region 1 second primer comprises a target hybridizing region 18-35 nucleobases in length, wherein the target hybridizing region is contained within SEQ ID NO: 140 or an RNA equivalent or a DNA/RNA chimeric thereof and contains a nucleotide sequence differing by no more than 0, 1, 2, 3, 4, or nucleotides from the nucleotide sequence of SEQ ID NO: 141 or an RNA equivalent or a DNA/RNA chimeric thereof. In some embodiments, the SARS-CoV-2 region 1 second primer comprises the nucleotide sequence of SEQ ID NO: 195, 196, or 197 or an RNA equivalent or a DNA/RNA chimeric thereof. In some embodiments, the SARS-CoV-2 region 1 second primer consists essentially of the nucleotide sequence of SEQ ID NO: 195, 196, or 197 or an RNA equivalent or a DNA/RNA chimeric thereof. In some embodiments, the SARS-CoV-2 region 1 second primer consists of the nucleotide sequence of SEQ ID NO: 195, 196, or 197 or an RNA equivalent or a DNA/RNA chimeric thereof. In some embodiments, the SARS-CoV-2 region 1 second primer comprises a nucleotide sequence having no more than 0, 1, 2, 3, 4, or mismatches from the nucleotide sequence of SEQ ID NO: 195, 196, or 197 or an RNA equivalent or a DNA/RNA chimeric thereof. In some embodiments, the SARS-CoV-2 region 1 second primer comprises a nucleotide sequence having at least 80%, at least 85% at least 90%, or at least 95% homology to the nucleotide sequence of SEQ ID NO: 195, 196, or 197 or an RNA equivalent or a DNA/RNA chimeric thereof. A SARS-CoV-2 region 1 second primer can have 1 or more modified nucleotides or nucleotide analogs. In some embodiments, two different second SARS-CoV-2 region 1 primers are used in a TMA reaction.

In some embodiments, a SARS-CoV-2 region 1 second primer comprises a SARS-CoV-2 region 1 promoter primer. In some embodiments, the SARS-CoV-2 region 1 promoter primer comprises the nucleotide sequence of SEQ ID NO: 167, 168, or 169 or an RNA equivalent or a DNA/RNA chimeric thereof (Table 3b). In some embodiments, the SARS-CoV-2 region 1 promoter primer consists essentially of the nucleotide sequence of SEQ ID NO: 167, 168, or 169 or an RNA equivalent or a DNA/RNA chimeric thereof. In some embodiments, the SARS-CoV-2 region 1 promoter primer consists of the nucleotide sequence of SEQ ID NO: 167, 168, or 169 or an RNA equivalent or a DNA/RNA chimeric thereof. In some embodiments, the SARS-CoV-2 region 1 promoter primer comprises a nucleotide sequence having no more than 0, 1, 2, 3, 4, or 5 mismatches from the nucleotide sequence of SEQ ID NO: 167, 168, or 169 or an RNA equivalent or a DNA/RNA chimeric thereof. In some embodiments, the SARS-CoV-2 region 1 promoter primer comprises a nucleotide sequence having at least 80%, at least 85% at least 90%, or at least 95% homology to the nucleotide sequence of SEQ ID NO: 167, 168, or 169 or an RNA equivalent or a DNA/RNA chimeric thereof. A SARS-CoV-2 region 1 promoter primer can have 1 or more modified nucleotides or nucleotide analogs. In some embodiments, two different SARS-CoV-2 region 1 promoter primers are used in a TMA reaction.

TABLE 3b
SARS-CoV-2 region 1 second/promoter primer sequences

| SEQ ID NO: | Sequence (5′→3′) |
|---|---|
| 140 | gagagttgaaaggcacatttggttgcattcatt |
| 141 | gagttgaaaggcacattt |
| 195 | GAGTTGAAAGGCACATTTGGTTG |
| 167 | AATTTAATACGACTCACTATAGGGAGAGAGTTGAAAGGCACATTTGGTTG |
| 196 | GAGAGTTGAAAGGCACATTTGGTTGCATTCATT |
| 168 | AATTTAATACGACTCACTATAGGGAGAGAGAGTTGAAAGGCACATTTGGTTGCATT |
| | CATT |
| 197 | GAGAGTTGAAAGGCACATTTGGTTGCATTC |
| 169 | AATTTAATACGACTCACTATAGGGAGAGAGAGTTGAAAGGCACATTTGGTTGCATT |
| | C |

In some embodiments, a SARS-CoV-2 region 1 probe comprises a target hybridizing region 20-30 nucleobases in length, wherein the target hybridizing region is contained within SEQ ID NO: 135, 136, or 137 or a DNA equivalent, a DNA/RNA chimeric, and/or a complement thereof and contains the nucleotide sequence of SEQ ID NO: 138 or 139 or a DNA equivalent, a DNA/RNA chimeric, and/or a complement thereof (Table 3c). In some embodiments, a SARS-CoV-2 region 1 probe comprises a target hybridizing region 20-30 nucleobases in length, wherein the target hybridizing region is contained within SEQ ID NO: 135, 136, or 137 or a DNA equivalent, a DNA/RNA chimeric, and/or a complement thereof and contains a nucleotide sequence differing by no more than 0, 1, 2, 3, 4, or 5 nucleotides from the nucleotide sequence of SEQ ID NO: 138 or 139 or a DNA equivalent, a DNA/RNA chimeric, and/or a complement thereof. In some embodiments, the SARS-CoV-2 region 1 probe comprises the nucleotide sequence of SEQ ID NO: 244, 245, or 246 or a DNA equivalent, a DNA/RNA chimeric, and/or a complement thereof. In some embodiments, the SARS-CoV-2 region 1 probe consists essentially of the nucleotide sequence of SEQ ID NO: 244, 245, or 246 or a DNA equivalent, a DNA/RNA chimeric, and/or a complement thereof. In some embodiments, the SARS-CoV-2 region 1 probe consists of the nucleotide sequence of SEQ ID NO: 244, 245, or 246 or a DNA equivalent, a DNA/RNA chimeric, and/or a complement thereof. In some embodiments, the SARS-CoV-2 region 1 probe comprises a nucleotide sequence having no more than 0, 1, 2, 3, 4, or 5 mismatches from the nucleotide sequence of SEQ ID NO: 244, 245, or 246 or a DNA equivalent, a DNA/RNA chimeric, and/or a complement thereof. In some embodiments, the SARS-CoV-2 region 1 probe comprises a nucleotide sequence having at least 80%, at least 85% at least 90%, or at least 95% homology to the nucleotide sequence of SEQ ID NO: 244, 245, or 246 or a DNA equivalent, a DNA/RNA chimeric, and/or a complement thereof. A SARS-CoV-2 region 1 probe can have 1 or more modified nucleotides or nucleotide analogs.

In some embodiments, a SARS-CoV-2 region 1 probe comprises a SARS-CoV-2 region 1 torch. In some embodiments, the SARS-CoV-2 region 1 torch comprises the nucleotide sequence of SEQ ID NO: 232, 233, 234, 266, 267, 268, 269, 270, 271, 272, 273, 274, or 275, or a DNA equivalent, a DNA/RNA chimeric, and/or a complement thereof (Table 3c). In some embodiments, the SARS-CoV-2 region 1 torch consists essentially of the nucleotide sequence of SEQ ID NO: 232, 233, 234, 266, 267, 268, 269, 270, 271, 272, 273, 274, or 275, or a DNA equivalent, a DNA/RNA chimeric, and/or a complement thereof. In some embodiments, the SARS-CoV-2 region 1 torch consists of the nucleotide sequence of SEQ ID NO: 232, 233, 234, 266, 267, 268, 269, 270, 271, 272, 273, 274, or 275, or a DNA equivalent, a DNA/RNA chimeric, and/or a complement thereof. In some embodiments, the SARS-CoV-2 region 1 torch comprises a nucleotide sequence having no more than 0, 1, 2, 3, 4, or 5 mismatches from the nucleotide sequence of SEQ ID NO: 232, 233, 234, 266, 267, 268, 269, 270, 271, 272, 273, 274, or 275, or a DNA equivalent, a DNA/RNA chimeric, and/or a complement thereof. In some embodiments, the SARS-CoV-2 region 1 torch comprises a nucleotide sequence having at least 80%, at least 85% at least 90%, or at least 95% homology to the nucleotide sequence of SEQ ID NO: 232, 233, 234, 266, 267, 268, 269, 270, 271, 272, 273, 274, or 275, or a DNA equivalent, a DNA/RNA chimeric, and/or a complement thereof. A SARS-CoV-2 region 1 torch can have 1 or more modified nucleotides or nucleotide analogs.

In some embodiments, the SARS-CoV-2 Region 1 probe comprises a nucleotide sequence contained in SEQ ID NO: 135 and comprising SEQ ID NO: 244. The SARS-CoV-2 Region 1 probe can further comprise a non-target specific sequence 0-5 residues in length at the 5′ end and/or a non-target specific sequence 0-7 residues at the 3′ end. In some embodiments, the non-target specific sequence is complementary with another sequence in the probe such that the probe forms an intramolecular double strand region (stem-loop structure) when the probe is not hybridized with a target nucleic acid. In some embodiments, a SARS-CoV-2 Region 1 probe comprising a nucleotide sequence contained in SEQ ID NO: 135 and comprising SEQ ID NO: 244 comprises SEQ ID NO: 244, SEQ ID NO: 234, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, or SEQ ID NO: 275 or a DNA equivalent, a DNA/RNA chimeric, and/or a complement thereof. The SARS-CoV-2 Region 1 probe can have 1 or more modified nucleotides or nucleotide analogs (e.g., 5-methyl C, 2′-OMe, inosine, etc).

In some embodiments, the SARS-CoV-2 Region 1 probe comprises a nucleotide sequence consisting of the nucleotide sequence of SEQ ID NO:244.

In some embodiments, the SARS-CoV-2 Region 1 probe comprises a nucleotide sequence consisting of the nucleotide sequence of SEQ ID NO:244, and a non-target specific sequence 0-5 residues in length at the 5′ end and/or a non-target specific sequence 0-7 residues at the 3′ end. In some embodiments, the non-target specific sequence is complementary with another sequence in the probe such that the probe forms an intramolecular double strand region (stem structure) when the probe is not hybridized with a target nucleic acid.

In some embodiments, the SARS-CoV-2 Region 1 probe comprises a nucleotide sequence consisting of the nucleotide sequence of SEQ ID NO:244, and a non-target specific sequence 0-2 residues in length at the 5′ end and a non-target specific sequence 5-7 residues at the 3′ end. In some embodiments, the non-target specific sequence at the 3′ end is complementary with another sequence in the probe such that the probe forms an intramolecular double strand region (stem structure) when the probe is not hybridized with a target nucleic acid.

In some embodiments, the SARS-CoV-2 Region 1 probe comprises a nucleotide sequence consisting of the nucleotide sequence of SEQ ID NO:244, and a non-target specific sequence 1-5 residues in length at the 5′ end and a non-target specific sequence 0-6 residues at the 3′ end. In some embodiments, the non-target specific sequence at the 3′ end is complementary with another sequence in the probe such that the probe forms an intramolecular double strand region (stem structure) when the probe is not hybridized with a target nucleic acid.

In some embodiments, the SARS-CoV-2 Region 1 probe comprises or consists of the nucleotide sequence of SEQ ID NO:271, wherein NNNNN and are non-target specific sequences, wherein each N is independently present or absent and if present is independently A, G, C, T, or U, wherein 4-8 residues at or near the 3′ end of the probe and/or 4-8 residues at or near the 5′ end of the probe form an intramolecular double strand region (stem structure) when the probe is not hybridized with a target nucleic acid.

In some embodiments, the SARS-CoV-2 Region 1 probe comprises or consists of the nucleotide sequence of SEQ ID NO:272, wherein NNNSS and VRDHBSS are non-target specific sequences, wherein each N is independently present or absent and if present is independently A, C, G, T or U (any residue), V is present or absent and if present is A, C, or G (i.e., not T or U), R is present or absent and if present is A or G (i.e., a purine), D is present or absent and if present is A, G, T or U (i.e., not C), H is present or absent and if present is A, C, T or U (i.e., not G), B is present or absent and if present is C, G, T or U (i.e., not A), and each S is independently is present or absent and if present is independently C or G, and wherein 4-8 residues at or near the 3′ end of the probe and/or 4-8 residues at or near the 5′ end of the probe form an intramolecular double strand region (stem structure) when the probe is not hybridized with a target nucleic acid.

In some embodiments, the SARS-CoV-2 Region 1 probe comprises or consists of the nucleotide sequence of SEQ ID NO:273, wherein SASSS and VRDHBSS are non-target specific sequences, wherein each S is independently is present or absent and if present is independently C or G, V is present or absent and if present is A, C, or G (i.e., not T or U), R is present or absent and if present is A or G (i.e., a purine), D is present or absent and if present is A, G, T or U (i.e., not C), His present or absent and if present is A, C, T or U (i.e., not G), B is present or absent and if present is C, G, T or U (i.e., not A), and wherein 4-8 residues at or near the 3′ end of the probe and/or 4-8 residues at or near the 5′ end of the probe form an intramolecular double strand region (stem structure) when the probe is not hybridized with a target nucleic acid.

In some embodiments, the SARS-CoV-2 Region 1 probe comprises or consists of the nucleotide sequence of SEQ ID NO:274, wherein N₀₋₅ and N₀₋₅ S₀₋₁ are each non-target specific sequences, wherein each N is independently A, G, C, T, or U and S is G or C, and wherein 4-8 residues at or near the 3′ end of the probe and/or 4-8 residues at or near the 5′ end of the probe form an intramolecular double strand region (stem structure) when the probe is not hybridized with a target nucleic acid.

In some embodiments, the SARS-CoV-2 Region 1 probe comprises or consists of the nucleotide sequence of SEQ ID NO:275, wherein N₀₋₅ and N₀₋₅ SS are non-target specific sequences, wherein each N is independently A, G, C, T, or U and each S is independently G or C, and wherein 4-8 residues at or near the 3′ end of the probe and/or 4-8 residues at or near the end of the probe form an intramolecular double strand region (stem structure) when the probe is not hybridized with a target nucleic acid.

In some embodiments, the SARS-CoV-2 Region 1 probe comprises or consists of the nucleotide sequence of SEQ ID NO: 234, SEQ ID NO: 266, SEQ ID NO: 267, SEQ ID NO: 268, SEQ ID NO: 267, or SEQ ID NO: 270 or a DNA equivalent, a DNA/RNA chimeric, and/or a complement thereof.

Any of the described SARS-CoV-2 Region 1 probes can have 1 or more modified nucleotides or nucleotide analogs (e.g., 5-methyl C, 2′-OMe, inosine, etc).

TABLE 3c
SARS-CoV-2 region 1 probe sequences

| SEQ ID NO: | Sequence (5′→3′) |
|---|---|
| 135 | gcnnganggcnnnangggnagaanncgancngncn |
| | anccagnngcgncaccaa (n = t or u) |
| 136 | cgaucugucuauccaguugcgucacc |
| 138 | gaucugucuauccaguugcguc |
| 137 | cgaucugucuauccaguugcgucaccaa |
| 139 | ucugucuauccaguugcguc |
| 245 | CGAUCUGUCUAUCCAGUUGCGUCAC |
| 232 | CGAUCUGUCUAUCCAGUUGCGUCACAUCG |
| 246 | GAUCUGUCUAUCCAGUUGCGUCACC |
| 233 | GAUCUGUCUAUCCAGUUGCGUCACCGAUC |
| 244 | CGAUCUGUCUAUCCAGUUGCGUC |
| 234 | CGAUCUGUCUAUCCAGUUGCGUCGAUCG |
| 266 | GCGAUCUGUCUAUCCAGUUGCGUCGAUCGC |
| 267 | GACGCCGAUCUGUCUAUCCAGUUGCGUC |
| 268 | CGAUCUGUCUAUCCAGUUGCGUCCAGAUCG |
| 269 | CGCGAUCUGUCUAUCCAGUUGCGUCGAUCGCG |
| 270 | GCGAUCUGUCUAUCCAGUUGCGUCAGAUCGC |
| 271 | NNNNNCGAUCUGUCUAUCCAGUUGCGUCNNNNNNN |
| 272 | NNNSSCGAUCUGUCUAUCCAGUUGCGUCVRDHBSS |
| 273 | SASSSCGAUCUGUCUAUCCAGUUGCGUCVRDHBSS |
| 274 | N₀₋₅CGAUCUGUCUAUCCAGUUGCGUCN₀₋₅S₀₋₁ |
| 275 | N₀₋₅CGAUCUGUCUAUCCAGUUGCGUCN₀₋₅SS |

A SARS-CoV-2 region 1 primer set includes at least one first primer and at least 1 second primer as described above. In some embodiments, a SARS-CoV-2 region 1 primer set includes at least one first primer and at least 2 different second primers.

F. SARS-CoV-2 Region 2

In some embodiments, a SARS-CoV-2 region 2 first primer comprises a target hybridizing region 16-30 nucleobases in length, wherein the target hybridizing region is contained within SEQ ID NO: 143 or an RNA equivalent or a DNA/RNA chimeric thereof and contains the nucleotide sequence of SEQ ID NO: 144 or an RNA equivalent or a DNA/RNA chimeric thereof (Table 3d). In some embodiments, a SARS-CoV-2 region 2 first primer comprises a target hybridizing region 16-30 nucleobases in length, wherein the target hybridizing region is contained within SEQ ID NO: 143 or an RNA equivalent or a DNA/RNA chimeric thereof and contains a nucleotide sequence differing by no more than 0, 1, 2, 3, 4, or nucleotides from the nucleotide sequence of SEQ ID NO: 144 or an RNA equivalent or a DNA/RNA chimeric thereof. In some embodiments, the SARS-CoV-2 region 2 first primer comprises the nucleotide sequence of SEQ ID NO: 128, 129, 130, or 131 or an RNA equivalent or a DNA/RNA chimeric thereof. In some embodiments, the SARS-CoV-2 region 2 first primer consists essentially of the nucleotide sequence of SEQ ID NO: 128, 129, 130, or 131 or an RNA equivalent or a DNA/RNA chimeric thereof. In some embodiments, the SARS-CoV-2 region 2 first primer consists of the nucleotide sequence of SEQ ID NO: 128, 129, 130, or 131 or an RNA equivalent or a DNA/RNA chimeric thereof. In some embodiments, the SARS-CoV-2 region 2 first primer comprises a nucleotide sequence having no more than 0, 1, 2, 3, 4, or 5 mismatches from the nucleotide sequence of SEQ ID NO: 128, 129, 130, or 131 or an RNA equivalent or a DNA/RNA chimeric thereof. In some embodiments, the SARS-CoV-2 region 2 first primer comprises a nucleotide sequence having at least 80%, at least 85% at least 90%, or at least 95% homology to the nucleotide sequence of SEQ ID NO: 128, 129, 130, or 131 or an RNA equivalent or a DNA/RNA chimeric thereof. A SARS-CoV-2 region 2 first primer can have 1 or more modified nucleotides or nucleotide analogs. In some embodiments, the SARS-CoV-2 region 2 first primer is an NT7 primer.

TABLE 3d
SARS-CoV-2 region 2 NT7 primer sequences

| SEQ ID NO: | Sequence (5′→3′) |
|---|---|
| 143 | gctgctcggtatatgagatctctc |
| 144 | ctcggtatatgagatc |
| 127 | GCTCGGTATATGAGATCTC |
| 128 | GCTGCTCGGTATATGAGATC |
| 129 | CTGCTCGGTATATGAGATC |
| 130 | GCTCGGTATATGAGATCTC |
| 131 | CTCGGTATATGAGATCTCTC |

In some embodiments, a SARS-CoV-2 region 2 second primer comprises a target hybridizing region 11-35 nucleobases in length, wherein the target hybridizing region is contained within SEQ ID NO: 147, 148, or 149 or an RNA equivalent or a DNA/RNA chimeric thereof and contains the nucleotide sequence of SEQ ID NO: 150, 151, or 152 or an RNA equivalent or a DNA/RNA chimeric thereof (Table 3e). In some embodiments, a SARS-CoV-2 region 2 second primer comprises a target hybridizing region 11-35 nucleobases in length, wherein the target hybridizing region is contained within SEQ ID NO: 147, 148, or 149 or an RNA equivalent or a DNA/RNA chimeric thereof and contains a nucleotide sequence differing by no more than 0, 1, 2, 3, 4, or 5 nucleotides from the nucleotide sequence of SEQ ID NO: 150, 151, or 152 or an RNA equivalent or a DNA/RNA chimeric thereof. In some embodiments, the SARS-CoV-2 region 2 second primer comprises the nucleotide sequence of SEQ ID NO: 198, 199, 200, 201, 202, 203, 204, 205, 206, 207, 208, or 209 or an RNA equivalent or a DNA/RNA chimeric thereof. In some embodiments, the SARS-CoV-2 region 2 second primer consists essentially of the nucleotide sequence of SEQ ID NO: 198, 199, 200, 201, 202, 203, 204, 205, 206, 207, 208, or 209 or an RNA equivalent or a DNA/RNA chimeric thereof. In some embodiments, the SARS-CoV-2 region 2 second primer consists of the nucleotide sequence of SEQ ID NO: 198, 199, 200, 201, 202, 203, 204, 205, 206, 207, 208, or 209 or an RNA equivalent or a DNA/RNA chimeric thereof. In some embodiments, the SARS-CoV-2 region 2 second primer comprises a nucleotide sequence having no more than 0, 1, 2, 3, 4, or 5 mismatches from the nucleotide sequence of SEQ ID NO: 198, 199, 200, 201, 202, 203, 204, 205, 206, 207, 208, or 209 or an RNA equivalent or a DNA/RNA chimeric thereof. In some embodiments, the SARS-CoV-2 region 2 second primer comprises a nucleotide sequence having at least 80%, at least 85% at least 90%, or at least 95% homology to the nucleotide sequence of SEQ ID NO: 198, 199, 200, 201, 202, 203, 204, 205, 206, 207, 208, or 209 or an RNA equivalent or a DNA/RNA chimeric thereof. A SARS-CoV-2 region 2 second primer can have 1 or more modified nucleotides or nucleotide analogs. In some embodiments, two different second SARS-CoV-2 region 2 primers are used in a TMA reaction.

In some embodiments, a SARS-CoV-2 region 2 second primer comprises a SARS-CoV-2 region 2 promoter primer. In some embodiments, the SARS-CoV-2 region 2 promoter primer comprises the nucleotide sequence of SEQ ID NO: 170, 171, 172, 173, 174, 175, 176, 177, 178, 179, 180, or 181 or an RNA equivalent or a DNA/RNA chimeric thereof (Table 3e). In some embodiments, the SARS-CoV-2 region 2 promoter primer consists essentially of the nucleotide sequence of SEQ ID NO: 170, 171, 172, 173, 174, 175, 176, 177, 178, 179, 180, or 181 or an RNA equivalent or a DNA/RNA chimeric thereof. In some embodiments, the SARS-CoV-2 region 2 promoter primer consists of the nucleotide sequence of SEQ ID NO: 170, 171, 172, 173, 174, 175, 176, 177, 178, 179, 180, or 181 or an RNA equivalent or a DNA/RNA chimeric thereof. In some embodiments, the SARS-CoV-2 region 2 promoter primer comprises a nucleotide sequence having no more than 0, 1, 2, 3, 4, or 5 mismatches from the nucleotide sequence of SEQ ID NO: 170, 171, 172, 173, 174, 175, 176, 177, 178, 179, 180, or 181 or an RNA equivalent or a DNA/RNA chimeric thereof. In some embodiments, the SARS-CoV-2 region 2 promoter primer comprises a nucleotide sequence having at least 80%, at least 85% at least 90%, or at least 95% homology to the nucleotide sequence of SEQ ID NO: 170, 171, 172, 173, 174, 175, 176, 177, 178, 179, 180, or 181 or an RNA equivalent or a DNA/RNA chimeric thereof. A SARS-CoV-2 region 2 promoter primer can have 1 or more modified nucleotides or nucleotide analogs. In some embodiments, two different SARS-CoV-2 region 2 promoter primers are used in a TMA reaction.

TABLE 3e
SARS-CoV-2 region 2 second/promoter primer sequences

| SEQ ID NO: | Sequence (5′→3′) |
|---|---|
| 147 | gaagtaagataaccattatacgctgtaacagcatcaggtgaagaaacag |
| 150 | gtaacagcatcaggtg |
| 148 | ccattatacgctgtaacagcatcaggtgaagaaacag |
| 151 | gtaacagcatcaggtgaag |
| 149 | gaagtaagataaccattatacgctgtaacagcatcag |
| 152 | gctgtaacagcatc |
| 153 | cattatacgct |
| 198 | GTAACAGCATCAGGTGAAGAAACA |
| 170 | AATTTAATACGACTCACTATAGGGAGAGTAACAGCATCAGGTGAAGAAACA |
| 199 | CTGTAACAGCATCAGGTGAAGAAAC |
| 171 | AATTTAATACGACTCACTATAGGGAGACTGTAACAGCATCAGGTGAAGAAAC |
| 200 | GCTGTAACAGCATCAGGTGAAGAAAC |
| 172 | AATTTAATACGACTCACTATAGGGAGAGCTGTAACAGCATCAGGTGAAGAAAC |
| 201 | CATTATACGCTGTAACAGCATCAGGTGAAGAAAC |
| 173 | AATTTAATACGACTCACTATAGGGAGACATTATACGCTGTAACAGCATCAGGTGAA |
| | GAAAC |
| 202 | TACGCTGTAACAGCATCAGGTGAAGAAA |
| 174 | AATTTAATACGACTCACTATAGGGAGATACGCTGTAACAGCATCAGGTGAAGAAA |
| 203 | CCATTATACGCTGTAACAGCATCAGGTGAAG |
| 175 | AATTTAATACGACTCACTATAGGGAGACCATTATACGCTGTAACAGCATCAGGTGA |
| | AG |
| 206 | GCTGTAACAGCATCAGGTGAAGAAACAG |
| 178 | AATTTAATACGACTCACTATAGGGAGAGCTGTAACAGCATCAGGTGAAGAAACAG |
| 204 | CATTATACGCTGTAACAGCATCAGGTG |
| 176 | AATTTAATACGACTCACTATAGGGAGACATTATACGCTGTAACAGCATCAGGTG |
| 205 | CATTATACGCTGTAACAGCATC |
| 177 | AATTTAATACGACTCACTATAGGGAGACATTATACGCTGTAACAGCATC |
| 207 | GAAGTAAGATAACCATTATACGCT |
| 179 | AATTTAATACGACTCACTATAGGGAGAGAAGTAAGATAACCATTATACGCT |
| 208 | GAAGTAAGATAACCATTATACGCTGTAACAGCATCAG |
| 180 | AATTTAATACGACTCACTATAGGGAGAGAAGTAAGATAACCATTATACGCTGTAAC |
| | AGCATCAG |
| 209 | GAAGTAAGATAACCATTATACGCTGTAACAGCATC |
| 181 | AATTTAATACGACTCACTATAGGGAGAGAAGTAAGATAACCATTATACGCTGTAAC |
| | AGCATC |

In some embodiments, a SARS-CoV-2 region 2 probe comprises a target hybridizing region 14-30 nucleobases in length, wherein the target hybridizing region is contained within SEQ ID NO: 142 or a DNA equivalent, a DNA/RNA chimeric, and/or a complement thereof and contains the nucleotide sequence of SEQ ID NO: 145 or 146 or a DNA equivalent, a DNA/RNA chimeric, and/or a complement thereof (Table 30. In some embodiments, a SARS-CoV-2 region 2 probe comprises a target hybridizing region 14-30 nucleobases in length, wherein the target hybridizing region is contained within SEQ ID NO: 142 or a DNA equivalent, a DNA/RNA chimeric, and/or a complement thereof and contains a nucleotide sequence differing by no more than 0, 1, 2, 3, 4, or 5 nucleotides from the nucleotide sequence of SEQ ID NO: 145 or 146 or a DNA equivalent, a DNA/RNA chimeric, and/or a complement thereof. In some embodiments, the SARS-CoV-2 region 2 probe comprises the nucleotide sequence of SEQ ID NO: 247, 248, 249, 250, 251, 252, or 258 or a DNA equivalent, a DNA/RNA chimeric, and/or a complement thereof. In some embodiments, the SARS-CoV-2 region 2 probe consists essentially of the nucleotide sequence of SEQ ID NO: 247, 248, 249, 250, 251, 252, or 258 or a DNA equivalent, a DNA/RNA chimeric, and/or a complement thereof. In some embodiments, the SARS-CoV-2 region 2 probe consists of the nucleotide sequence of SEQ ID NO: 247, 248, 249, 250, 251, 252, or 258 or a DNA equivalent, a DNA/RNA chimeric, and/or a complement thereof. In some embodiments, the SARS-CoV-2 region 2 probe comprises a nucleotide sequence having no more than 0, 1, 2, 3, 4, or 5 mismatches from the nucleotide sequence of SEQ ID NO: 247, 248, 249, 250, 251, 252, or 258 or a DNA equivalent, a DNA/RNA chimeric, and/or a complement thereof. In some embodiments, the SARS-CoV-2 region 2 probe comprises a nucleotide sequence having at least 80%, at least 85% at least 90%, or at least 95% homology to the nucleotide sequence of SEQ ID NO: 247, 248, 249, 250, 251, 252, or 258 or a DNA equivalent, a DNA/RNA chimeric, and/or a complement thereof. A SARS-CoV-2 region 2 probe can have 1 or more modified nucleotides or nucleotide analogs.

In some embodiments, a SARS-CoV-2 region 2 probe comprises a SARS-CoV-2 region 2 torch. In some embodiments, the SARS-CoV-2 region 2 torch comprises the nucleotide sequence of SEQ ID NO: 235, 236, 237, 238, 239, 240, or 257 or a DNA equivalent, a DNA/RNA chimeric, and/or a complement thereof (Table 30. In some embodiments, the SARS-CoV-2 region 2 torch consists essentially of the nucleotide sequence of SEQ ID NO: 235, 236, 237, 238, 239, 240, or 257 or a DNA equivalent, a DNA/RNA chimeric, and/or a complement thereof. In some embodiments, the SARS-CoV-2 region 2 torch consists of the nucleotide sequence of SEQ ID NO: 235, 236, 237, 238, 239, 240, or 257 or a DNA equivalent, a DNA/RNA chimeric, and/or a complement thereof. In some embodiments, the SARS-CoV-2 region 2 torch comprises a nucleotide sequence having no more than 0, 1, 2, 3, 4, or 5 mismatches from the nucleotide sequence of SEQ ID NO: 235, 236, 237, 238, 239, 240, or 257 or a DNA equivalent, a DNA/RNA chimeric, and/or a complement thereof. In some embodiments, the SARS-CoV-2 region 2 torch comprises a nucleotide sequence having at least 80%, at least 85% at least 90%, or at least 95% homology to the nucleotide sequence of SEQ ID NO: 235, 236, 237, 238, 239, 240, or 257 or a DNA equivalent, a DNA/RNA chimeric, and/or a complement thereof. A SARS-CoV-2 region 2 torch can have 1 or more modified nucleotides or nucleotide analogs.

TABLE 3f
SARS-CoV-2 region 2 probe sequences

| SEQ ID NO: | Sequence (5′→3′) |
|---|---|
| 142 | gcngcncggnanangagancncncaaagngccagcna |
| | cagnnncngnnnc (n = t or u) |
| 145 | ucucaaagugccagcuacaguuuc |
| 146 | gugccagcuacagu |
| 247 | GCUCAAAGUGCCAGCUACAGU |
| 235 | GCUCAAAGUGCCAGCUACAGUUGAGC |
| 248 | CUCAAAGUGCCAGCUACAGU |
| 236 | CUCAAAGUGCCAGCUACAGUUGAG |
| 249 | CAAAGUGCCAGCUACAGUUUC |
| 237 | CAAAGUGCCAGCUACAGUUUCCUUUG |
| 250 | CAAAGUGCCAGCUACAGUUUCUG |
| 238 | CAAAGUGCCAGCUACAGUUUCUGCUUUG |
| 251 | AGUGCCAGCUACAGUUUCUG |
| 239 | AGUGCCAGCUACAGUUUCUGCACU |
| 252 | GUGCCAGCUACAGUUUCUGUUUC |
| 240 | GUGCCAGCUACAGUUUCUGUUUCGCAC |
| 258 | CGCUCAAAGUGCCAGCUACAGU |
| 257 | CGCUCAAAGUGCCAGCUACAGUUGAGCG |

A SARS-CoV-2 region 2 primer set includes at least one first primer and at least 1 second primer as described above. In some embodiments, a SARS-CoV-2 region 2 primer set includes at least one first primer and at least 2 different second primers.

G. SARS-CoV-2 Region 3

In some embodiments, a SARS-CoV-2 region 3 first primer comprises a target hybridizing region 18-30 nucleobases in length, wherein the target hybridizing region is contained within SEQ ID NO: 155 or an RNA equivalent or a DNA/RNA chimeric thereof and contains the nucleotide sequence of SEQ ID NO: 156 or 157 or an RNA equivalent or a DNA/RNA chimeric thereof (Table 3g). In some embodiments, a SARS-CoV-2 region 3 first primer comprises a target hybridizing region 18-30 nucleobases in length, wherein the target hybridizing region is contained within SEQ ID NO: 155 or an RNA equivalent or a DNA/RNA chimeric thereof and contains a nucleotide sequence differing by no more than 0, 1, 2, 3, 4, or nucleotides from the nucleotide sequence of SEQ ID NO: 156 or 157 or an RNA equivalent or a DNA/RNA chimeric thereof. In some embodiments, the SARS-CoV-2 region 3 first primer comprises the nucleotide sequence of SEQ ID NO: 132, 133, or 134 or an RNA equivalent or a DNA/RNA chimeric thereof. In some embodiments, the SARS-CoV-2 region 3 first primer consists essentially of the nucleotide sequence of SEQ ID NO: 132, 133, or 134 or an RNA equivalent or a DNA/RNA chimeric thereof. In some embodiments, the SARS-CoV-2 region 3 first primer consists of the nucleotide sequence of SEQ ID NO: 132, 133, or 134 or an RNA equivalent or a DNA/RNA chimeric thereof. In some embodiments, the SARS-CoV-2 region 3 first primer comprises a nucleotide sequence having no more than 0, 1, 2, 3, 4, or 5 mismatches from the nucleotide sequence of SEQ ID NO: 132, 133, or 134 or an RNA equivalent or a DNA/RNA chimeric thereof. In some embodiments, the SARS-CoV-2 region 3 first primer comprises a nucleotide sequence having at least 80%, at least 85% at least 90%, or at least 95% homology to the nucleotide sequence of SEQ ID NO: 132, 133, or 134 or an RNA equivalent or a DNA/RNA chimeric thereof. A SARS-CoV-2 region 3 first primer can have 1 or more modified nucleotides or nucleotide analogs. In some embodiments, the SARS-CoV-2 region 3 first primer is an NT7 primer.

TABLE 3g
SARS-CoV-2 region 3 NT7 primer sequences

| SEQ ID NO: | Sequence (5′→3′) |
|---|---|
| 155 | cagggtgctgtagacataaacaagctt |
| 156 | ggtgctgtagacataaac |
| 157 | ctgtagacataaacaagctt |
| 132 | CAGGGTGCTGTAGACATAAAC |
| 133 | GGTGCTGTAGACATAAACAAGC |
| 134 | CTGTAGACATAAACAAGCTT |

In some embodiments, a SARS-CoV-2 region 3 second primer comprises a target hybridizing region 13-40 nucleobases in length, wherein the target hybridizing region is contained within SEQ ID NO: 160, 161, or 162 or an RNA equivalent or a DNA/RNA chimeric thereof and contains the nucleotide sequence of SEQ ID NO: 163, 164, 165, or 166 or an RNA equivalent or a DNA/RNA chimeric thereof (Table 3h). In some embodiments, a SARS-CoV-2 region 3 second primer comprises a target hybridizing region 13-40 nucleobases in length, wherein the target hybridizing region is contained within SEQ ID NO: 160, 161, or 162 or an RNA equivalent or a DNA/RNA chimeric thereof and contains a nucleotide sequence differing by no more than 0, 1, 2, 3, 4, or 5 nucleotides from the nucleotide sequence of SEQ ID NO: 163, 164, 165, or 166 or an RNA equivalent or a DNA/RNA chimeric thereof. In some embodiments, the SARS-CoV-2 region 3 second primer comprises the nucleotide sequence of SEQ ID NO: 210, 211, 212, 213, 214, 215, 216, 217, 218, 219, 220, 221, or 222 or an RNA equivalent or a DNA/RNA chimeric thereof. In some embodiments, the SARS-CoV-2 region 3 second primer consists essentially of the nucleotide sequence of SEQ ID NO: 210, 211, 212, 213, 214, 215, 216, 217, 218, 219, 220, 221, or 222 or an RNA equivalent or a DNA/RNA chimeric thereof. In some embodiments, the SARS-CoV-2 region 3 second primer consists of the nucleotide sequence of SEQ ID NO: 210, 211, 212, 213, 214, 215, 216, 217, 218, 219, 220, 221, or 222 or an RNA equivalent or a DNA/RNA chimeric thereof. In some embodiments, the SARS-CoV-2 region 3 second primer comprises a nucleotide sequence having no more than 0, 1, 2, 3, 4, or 5 mismatches from the nucleotide sequence of SEQ ID NO: 210, 211, 212, 213, 214, 215, 216, 217, 218, 219, 220, 221, or 222 or an RNA equivalent or a DNA/RNA chimeric thereof. In some embodiments, the SARS-CoV-2 region 3 second primer comprises a nucleotide sequence having at least 80%, at least 85% at least 90%, or at least 95% homology to the nucleotide sequence of SEQ ID NO: 210, 211, 212, 213, 214, 215, 216, 217, 218, 219, 220, 221, or 222 or an RNA equivalent or a DNA/RNA chimeric thereof. A SARS-CoV-2 region 3 second primer can have 1 or more modified nucleotides or nucleotide analogs. In some embodiments, two different second SARS-CoV-2 region 3 primers are used in a TMA reaction.

In some embodiments, a SARS-CoV-2 region 3 second primer comprises a SARS-CoV-2 region 3 promoter primer. In some embodiments, the SARS-CoV-2 region 3 promoter primer comprises the nucleotide sequence of SEQ ID NO: 182, 183, 184, 185, 186, 187, 188, 189, 190, 191, 192, 193, or 194 or an RNA equivalent or a DNA/RNA chimeric thereof (Table 3h). In some embodiments, the SARS-CoV-2 region 3 promoter primer consists essentially of the nucleotide sequence of SEQ ID NO: 182, 183, 184, 185, 186, 187, 188, 189, 190, 191, 192, 193, or 194 or an RNA equivalent or a DNA/RNA chimeric thereof. In some embodiments, the SARS-CoV-2 region 3 promoter primer consists of the nucleotide sequence of SEQ ID NO: 182, 183, 184, 185, 186, 187, 188, 189, 190, 191, 192, 193, or 194 or an RNA equivalent or a DNA/RNA chimeric thereof. In some embodiments, the SARS-CoV-2 region 3 promoter primer comprises a nucleotide sequence having no more than 0, 1, 2, 3, 4, or 5 mismatches from the nucleotide sequence of SEQ ID NO: 182, 183, 184, 185, 186, 187, 188, 189, 190, 191, 192, 193, or 194 or an RNA equivalent or a DNA/RNA chimeric thereof. In some embodiments, the SARS-CoV-2 region 3 promoter primer comprises a nucleotide sequence having at least 80%, at least 85% at least 90%, or at least 95% homology to the nucleotide sequence of SEQ ID NO: 182, 183, 184, 185, 186, 187, 188, 189, 190, 191, 192, 193, or 194 or an RNA equivalent or a DNA/RNA chimeric thereof. A SARS-CoV-2 region 3 promoter primer can have 1 or more modified nucleotides or nucleotide analogs. In some embodiments, two different SARS-CoV-2 region 3 promoter primers are used in a TMA reaction.

TABLE 3h
SARS-CoV-2 region 3 second/promoter primer sequences

| SEQ ID NO: | Sequence (5′→3′) |
|---|---|
| 160 | cttgagcagtagcaaaagctgcatatgatggaagggaactaaactctgaggctatagcttgtaagkktgccct |
| 163 | ctatagcttgtaagkktg |
| 164 | ctatagcttgtaa |
| 161 | cttgagcagtagcaaaagctgcatatgatggaagggaactaaac |
| 166 | aaaagctgcatatgatgga |
| 162 | aaactctgaggctatagcttgtaagkktgccct |
| 165 | gaggctatagcttgtaag |
| 210 | AGCAAAAGCTGCATATGATGGAAGGGAACTAAAC |
| 182 | AATTTAATACGACTCACTATAGGGAGAAGCAAAAGCTGCATATGATGGAAGGGAA |
| | CTAAAC |
| 211 | GCAAAAGCTGCATATGATGGAAGGGAAC |
| 183 | AATTTAATACGACTCACTATAGGGAGAGCAAAAGCTGCATATGATGGAAGGGAAC |
| 212 | CTTGAGCAGTAGCAAAAGCTGCATATGATGGAAGG |
| 184 | AATTTAATACGACTCACTATAGGGAGACTTGAGCAGTAGCAAAAGCTGCATATGAT |
| | GGAAGG |
| 222 | GCAAAAGCTGCATATGATGGAAGG |
| 194 | AATTTAATACGACTCACTATAGGGAGAGCAAAAGCTGCATATGATGGAAGG |
| 217 | CAGTAGCAAAAGCTGCATATGATGGAAGGG |
| 189 | AATTTAATACGACTCACTATAGGGAGACAGTAGCAAAAGCTGCATATGATGGAAG |
| | GG |
| 213 | GAGGCTATAGCTTGTAAGGTTGCCCT |
| 185 | AATTTAATACGACTCACTATAGGGAGAGAGGCTATAGCTTGTAAGGTTGCCCT |
| 214 | ACTCTGAGGCTATAGCTTGTAAGGTTGCCCT |
| 186 | AATTTAATACGACTCACTATAGGGAGAACTCTGAGGCTATAGCTTGTAAGGTTGCC |
| | CT |
| 215 | ACTCTGAGGCTATAGCTTGTAAGGTTG |
| 187 | AATTTAATACGACTCACTATAGGGAGAACTCTGAGGCTATAGCTTGTAAGGTTG |
| 216 | AAACTCTGAGGCTATAGCTTGTAAGGTTG |
| 188 | AATTTAATACGACTCACTATAGGGAGAAAACTCTGAGGCTATAGCTTGTAAGGTTG |
| 218 | CTATAGCTTGTAAGGTTGCCCT |
| 190 | AATTTAATACGACTCACTATAGGGAGACTATAGCTTGTAAGGTTGCCCT |
| 219 | GAGGCTATAGCTTGTAAGGTTG |
| 191 | AATTTAATACGACTCACTATAGGGAGAGAGGCTATAGCTTGTAAGGTTG |
| 220 | CTCTGAGGCTATAGCTTGTAAG |
| 192 | AATTTAATACGACTCACTATAGGGAGACTCTGAGGCTATAGCTTGTAAG |
| 221 | CTCTGAGGCTATAGCTTGTAA |
| 193 | AATTTAATACGACTCACTATAGGGAGACTCTGAGGCTATAGCTTGTAA |

In some embodiments, a SARS-CoV-2 region 3 probe comprises a target hybridizing region 22-35 nucleobases in length, wherein the target hybridizing region is contained within SEQ ID NO: 154 or 158 or a DNA equivalent, a DNA/RNA chimeric, and/or a complement thereof and contains the nucleotide sequence of SEQ ID NO: 159 or a DNA equivalent, a DNA/RNA chimeric, and/or a complement thereof (Table 3i). In some embodiments, a SARS-CoV-2 region 3 probe comprises a target hybridizing region 22-35 nucleobases in length, wherein the target hybridizing region is contained within SEQ ID NO: 154 or 158 or a DNA equivalent, a DNA/RNA chimeric, and/or a complement thereof and contains a nucleotide sequence differing by no more than 0, 1, 2, 3, 4, or 5 nucleotides from the nucleotide sequence of SEQ ID NO: 159 or a DNA equivalent, a DNA/RNA chimeric, and/or a complement thereof. In some embodiments, the SARS-CoV-2 region 3 probe comprises the nucleotide sequence of SEQ ID NO: 253, 254, or 255 or a DNA equivalent, a DNA/RNA chimeric, and/or a complement thereof. In some embodiments, the SARS-CoV-2 region 3 probe consists essentially of the nucleotide sequence of SEQ ID NO: 253, 254, or 255 or a DNA equivalent, a DNA/RNA chimeric, and/or a complement thereof. In some embodiments, the SARS-CoV-2 region 3 probe consists of the nucleotide sequence of SEQ ID NO: 253, 254, or 255 or a DNA equivalent, a DNA/RNA chimeric, and/or a complement thereof. In some embodiments, the SARS-CoV-2 region 3 probe comprises a nucleotide sequence having no more than 0, 1, 2, 3, 4, or 5 mismatches from the nucleotide sequence of SEQ ID NO: 253, 254, or 255 or a DNA equivalent, a DNA/RNA chimeric, and/or a complement thereof. In some embodiments, the SARS-CoV-2 region 3 probe comprises a nucleotide sequence having at least 80%, at least 85% at least 90%, or at least 95% homology to the nucleotide sequence of SEQ ID NO: 253, 254, or 255 or a DNA equivalent, a DNA/RNA chimeric, and/or a complement thereof. A SARS-CoV-2 region 3 probe can have 1 or more modified nucleotides or nucleotide analogs.

In some embodiments, a SARS-CoV-2 region 3 probe comprises a SARS-CoV-2 region 3 torch. In some embodiments, the SARS-CoV-2 region 3 torch comprises the nucleotide sequence of SEQ ID NO: 241, 242, or 243 or a DNA equivalent, a DNA/RNA chimeric, and/or a complement thereof (Table 3i). In some embodiments, the SARS-CoV-2 region 3 torch consists essentially of the nucleotide sequence of SEQ ID NO: 241, 242, or 243 or a DNA equivalent, a DNA/RNA chimeric, and/or a complement thereof. In some embodiments, the SARS-CoV-2 region 3 torch consists of the nucleotide sequence of SEQ ID NO: 241, 242, or 243 or a DNA equivalent, a DNA/RNA chimeric, and/or a complement thereof. In some embodiments, the SARS-CoV-2 region 3 torch comprises a nucleotide sequence having no more than 0, 1, 2, 3, 4, or 5 mismatches from the nucleotide sequence of SEQ ID NO: 241, 242, or 243 or a DNA equivalent, a DNA/RNA chimeric, and/or a complement thereof. In some embodiments, the SARS-CoV-2 region 3 torch comprises a nucleotide sequence having at least 80%, at least 85% at least 90%, or at least 95% homology to the nucleotide sequence of SEQ ID NO: 241, 242, or 243 or a DNA equivalent, a DNA/RNA chimeric, and/or a complement thereof. A SARS-CoV-2 region 3 torch can have 1 or more modified nucleotides or nucleotide analogs.

TABLE 3i
SARS-CoV-2 region 3 probe sequences

| SEQ ID NO: | Sequence (5′→3′) |
|---|---|
| 154 | cagggngcngnagacanaaacaagcnnngngaagaaa |
| | ngcnggacaacagggcaac (n = t or u) |
| 158 | gcuuugugaagaaaugcuggacaacagggcamm |
| 159 | gugaagaaaugcuggacaacag |
| 253 | GCUUUGUGAAGAAAUGCUGGACAACAG |
| 241 | GCUUUGUGAAGAAAUGCUGGACAACAGAAGC |
| 254 | UGUGAAGAAAUGCUGGACAACAGGG |
| 242 | UGUGAAGAAAUGCUGGACAACAGGGCACA |
| 255 | GUGAAGAAAUGCUGGACAACAG |
| 243 | GUGAAGAAAUGCUGGACAACAGUCAC |

A SARS-CoV-2 region 3 primer set includes at least one first primer and at least 1 second primer as described above. In some embodiments, a SARS-CoV-2 region 3 primer set includes at least one first primer and at least 2 different second primers.

### III. Target Capture Oligos

In some embodiments, the methods further include purifying the SARS-CoV-2, influenza A, and/or influenza B target nucleic acids from other components in the sample, e.g., before an amplification. Such purification may include methods of separating and/or concentrating organisms or components thereof, such as nucleic acid, contained in a sample from other sample components, or removing or degrading non-nucleic acid sample components, e.g., protein, carbohydrate, salt, lipid, etc. In some embodiments, a target nucleic acid is captured specifically or non-specifically and separated from other sample components. Non-specific target capture methods may involve selective precipitation of nucleic acids from a substantially aqueous mixture, adherence of nucleic acids to a support that is washed to remove other sample components, or other means of physically separating nucleic acids from a mixture that contains, or is suspected of containing, SARS-CoV-2, influenza A, and/or influenza B nucleic acid from other sample components.

Target capture typically occurs in a solution phase mixture that contains one or more target capture oligonucleotides (TCOs) that hybridize to the SARS-CoV-2, influenza A, and/or influenza B target sequence under hybridizing conditions. For embodiments comprising a capture probe tail, the SARS-CoV-2, influenza A, or influenza B-target nucleic acid:capture-probe complex is captured by adjusting the hybridization conditions so that the capture probe tail hybridizes to an immobilized probe. The immobilized probe can comprise particulate solid support, such as paramagnetic beads. Specific and non-specific target capture methods are also described, e.g., in U.S. Pat. No. 6,110,678 and International Patent Application Pub. No. WO 2008/016988, each incorporated by reference herein. SARS-CoV-2, influenza A, and/or influenza B TCOs are described below.

Isolation can follow capture, where, for example, the complex on the solid support is separated from other sample components. Isolation can be accomplished by any appropriate technique, e.g., washing a support associated with the SARS-CoV-2, influenza A, and/or influenza B target sequences one or more times (e.g., two or three times) to remove other sample components and/or unbound oligomer. In embodiments using a particulate solid support, such as paramagnetic beads, particles associated with the SARS-CoV-2, influenza A, and/or influenza B targets may be suspended in a washing solution and retrieved from the washing solution. Methods of retrieving include, but are not limited to, the use of magnetic attraction. To limit the number of handling steps, the SARS-CoV-2, influenza A, and/or influenza B target nucleic acids may be amplified by simply mixing the target sequences in the complex on the support with amplification oligomers and proceeding with amplification steps.

Sample preparation may also include pooling a plurality of samples into a single pooled batch. In some embodiments, pooling comprises combining an aliquot of two or more samples. In some embodiments, 2-200 samples are pooled.

A. Influenza A

In some embodiments, an influenza A target capture oligonucleotide comprises a target hybridizing region 26-30 nucleobases in length, wherein the target hybridizing region comprises the nucleotide sequence of SEQ ID NO: 51, 52, or 53 or an RNA equivalent, a DNA/RNA chimeric, and/or complement thereof (Table 1g). In some embodiments, an influenza A target capture oligonucleotide comprises a target hybridizing region 26-30 nucleobases in length, wherein the target hybridizing region comprises a nucleotide sequence differing by no more than 0, 1, 2, 3, 4, or 5 nucleotides from the nucleotide sequence of SEQ ID NO: 51, 52, or 53 or an RNA equivalent, a DNA/RNA chimeric, and/or complement thereof. In some embodiments, the influenza A target capture oligonucleotide comprises the nucleotide sequence of SEQ ID NO: 51, 52, or 53 or an RNA equivalent or a DNA/RNA chimeric thereof. In some embodiments, the target hybridizing region of the influenza A target capture oligonucleotide consists essentially of the nucleotide sequence of SEQ ID NO: 51, 52, or 53 or an RNA equivalent or a DNA/RNA chimeric thereof. In some embodiments, the target hybridizing region of the influenza A target capture oligonucleotide consists of the nucleotide sequence of SEQ ID NO: 51, 52, or 53 or an RNA equivalent or a DNA/RNA chimeric thereof In some embodiments, the influenza A target capture oligonucleotide comprises a nucleotide sequence having no more than 0, 1, 2, 3, 4, or 5 mismatches from the nucleotide sequence of SEQ ID NO: 51, 52, or 53 or an RNA equivalent or a DNA/RNA chimeric thereof. In some embodiments, the influenza A target capture oligonucleotide comprises a nucleotide sequence having at least 80%, at least 85% at least 90%, or at least 95% homology to the nucleotide sequence of SEQ ID NO: 51, 52, or 53 or an RNA equivalent or a DNA/RNA chimeric thereof. An influenza A target capture oligonucleotide can have 1 or more modified nucleotides or nucleotide analogs.

In some embodiments, an influenza A region 1 target capture oligonucleotide is linked to a TₙAₘ sequence, wherein n in an integer from 0 to 3 and m in an integer from 14 to 50. In some embodiments, n is 3 and m is 30. In some embodiments, an influenza target capture oligonucleotide comprises the nucleotide sequence of SEQ ID NO: 48, 49, or 50 or an RNA equivalent or a DNA/RNA chimeric thereof (Table 1g). In some embodiments, an influenza target capture oligonucleotide consists essentially of the nucleotide sequence of SEQ ID NO: 48, 49, or 50 or an RNA equivalent or a DNA/RNA chimeric thereof. In some embodiments, an influenza target capture oligonucleotide consists of the nucleotide sequence of SEQ ID NO: 48, 49, or 50 or an RNA equivalent or a DNA/RNA chimeric thereof. In some embodiments, an influenza target capture oligonucleotide comprises a nucleotide sequence having no more than 0, 1, 2, 3, 4, or 5 mismatches from the nucleotide sequence of SEQ ID NO: 48, 49, or 50 or an RNA equivalent or a DNA/RNA chimeric thereof. In some embodiments, an influenza target capture oligonucleotide comprises a nucleotide sequence having at least 80%, at least 85% at least 90%, or at least 95% homology to the nucleotide sequence of SEQ ID NO: 48, 49, or 50 or an RNA equivalent or a DNA/RNA chimeric thereof. An influenza A region 1 promoter primer can have 1 or more modified nucleotides or nucleotide analogs.

TABLE 1g
Influenza A Target Capture oligo sequences

| SEQ ID NO: | Sequence (5′→3′) |
|---|---|
| 51 | CUCACCGUGCCCAGUGAGCGAGGACU |
| 48 | CUCACCGUGCCCAGUGAGCGAGGACUTTTAAAAAAAAAAAAAAAAAAAAAAAAA |
| | AAAAA |
| 52 | GGUGCACUUGCCAGUUGCAUGGGCCUCAU |
| 49 | GGUGCACUUGCCAGUUGCAUGGGCCUCAUTTTAAAAAAAAAAAAAAAAAAAAAA |
| | AAAAAAAA |
| 53 | GCACUUGCCAGUUGCAUGGGCCUCAUAUAC |
| 50 | GCACUUGCCAGUUGCAUGGGCCUCAUAUACTTTAAAAAAAAAAAAAAAAAAAAA |
| | AAAAAAAAA |

B. Influenza B

In some embodiments, an influenza B target capture oligonucleotide comprises a target hybridizing region 25-30 nucleobases in length, wherein the target hybridizing region comprises the nucleotide sequence of SEQ ID NO: 111, 112, or 113 or an RNA equivalent, a DNA/RNA chimeric, and/or complement thereof (Table 2g). In some embodiments, an influenza B target capture oligonucleotide comprises a target hybridizing region 25-30 nucleobases in length, wherein the target hybridizing region comprises a nucleotide sequence differing by no more than 0, 1, 2, 3, 4, or 5 nucleotides from the nucleotide sequence of SEQ ID NO: 111, 112, or 113 or an RNA equivalent, a DNA/RNA chimeric, and/or complement thereof. In some embodiments, the influenza B target capture oligonucleotide comprises the nucleotide sequence of SEQ ID NO: 111, 112, or 113 or an RNA equivalent or a DNA/RNA chimeric thereof. In some embodiments, the target hybridizing region of the influenza B target capture oligonucleotide consists essentially of the nucleotide sequence of SEQ ID NO: 111, 112, or 113 or an RNA equivalent or a DNA/RNA chimeric thereof. In some embodiments, the target hybridizing region of the influenza B target capture oligonucleotide consists of the nucleotide sequence of SEQ ID NO: 111, 112, or 113 or an RNA equivalent or a DNA/RNA chimeric thereof. In some embodiments, the influenza B target capture oligonucleotide comprises a nucleotide sequence having no more than 0, 1, 2, 3, 4, or 5 mismatches from the nucleotide sequence of SEQ ID NO: 111, 112, or 113 or an RNA equivalent or a DNA/RNA chimeric thereof. In some embodiments, the influenza B target capture oligonucleotide comprises a nucleotide sequence having at least 80%, at least 85% at least 90%, or at least 95% homology to the nucleotide sequence of SEQ ID NO: 111, 112, or 113 or an RNA equivalent or a DNA/RNA chimeric thereof. An influenza B target capture oligonucleotide can have 1 or more modified nucleotides or nucleotide analogs.

In some embodiments, an influenza B region 1 target capture oligonucleotide is linked to a TₙAₘ sequence, wherein n in an integer from 0 to 3 and m in an integer from 14 to 50. In some embodiments, n is 3 and m is 30. In some embodiments, an influenza target capture oligonucleotide comprises the nucleotide sequence of SEQ ID NO: 108, 109, or 110 or an RNA equivalent or a DNA/RNA chimeric thereof (Table 2g). In some embodiments, an influenza target capture oligonucleotide consists essentially of the nucleotide sequence of SEQ ID NO: 108, 109, or 110 or an RNA equivalent or a DNA/RNA chimeric thereof. In some embodiments, an influenza target capture oligonucleotide consists of the nucleotide sequence of SEQ ID NO: 108, 109, or 110 or an RNA equivalent or a DNA/RNA chimeric thereof. In some embodiments, an influenza target capture oligonucleotide comprises a nucleotide sequence having no more than 0, 1, 2, 3, 4, or 5 mismatches from the nucleotide sequence of SEQ ID NO: 108, 109, or 110 or an RNA equivalent or a DNA/RNA chimeric thereof. In some embodiments, an influenza target capture oligonucleotide comprises a nucleotide sequence having at least 80%, at least 85% at least 90%, or at least 95% homology to the nucleotide sequence of SEQ ID NO: 108, 109, or 110 or an RNA equivalent or a DNA/RNA chimeric thereof. An influenza B region 1 promoter primer can have 1 or more modified nucleotides or nucleotide analogs.

TABLE 2g
Influenza B Target Capture oligo sequences

| SEQ ID NO: | Sequence (5′→3′) |
|---|---|
| 111 | CUGAUAUACAGAAAGCACUAAUUGG |
| 108 | CUGAUAUACAGAAAGCACUAAUUGGTTTAAAAAAAAAAAAAAAAAAAAAAAAAA |
| | AAAA |
| 112 | GUGGAGGAUGAAGAAGAUGGCCAUCG |
| 109 | GUGGAGGAUGAAGAAGAUGGCCAUCGTTTAAAAAAAAAAAAAAAAAAAAAAAAA |
| | AAAAA |
| 113 | GAGGAUGAAGAAGAUGGCCAUCGGAUC |
| 110 | GAGGAUGAAGAAGAUGGCCAUCGGAUCTTTAAAAAAAAAAAAAAAAAAAAAAAA |
| | AAAAAA |

C. SARS-CoV-2

In some embodiments, a SARS-CoV-2 target capture oligonucleotide comprises a target hybridizing region 22-30 nucleobases in length, wherein the target hybridizing region comprises the nucleotide sequence of SEQ ID NO: 228, 229, 230, 231, or 256 or an RNA equivalent, a DNA/RNA chimeric, and/or complement thereof (Table 3j). In some embodiments, a SARS-CoV-2 target capture oligonucleotide comprises a target hybridizing region 22-30 nucleobases in length, wherein the target hybridizing region comprises a nucleotide sequence differing by no more than 0, 1, 2, 3, 4, or 5 nucleotides from the nucleotide sequence of SEQ ID NO: 228, 229, 230, 231, or 256 or an RNA equivalent, a DNA/RNA chimeric, and/or complement thereof. In some embodiments, the SARS-CoV-2 target capture oligonucleotide comprises the nucleotide sequence of SEQ ID NO: 228, 229, 230, 231, or 256 or an RNA equivalent or a DNA/RNA chimeric thereof. In some embodiments, the target hybridizing region of the SARS-CoV-2 target capture oligonucleotide consists essentially of the nucleotide sequence of SEQ ID NO: 228, 229, 230, 231, or 256 or an RNA equivalent or a DNA/RNA chimeric thereof. In some embodiments, the target hybridizing region of the SARS-CoV-2 target capture oligonucleotide consists of the nucleotide sequence of SEQ ID NO: 228, 229, 230, 231, or 256 or an RNA equivalent or a DNA/RNA chimeric thereof. In some embodiments, the SARS-CoV-2 target capture oligonucleotide comprises a nucleotide sequence having no more than 0, 1, 2, 3, 4, or 5 mismatches from the nucleotide sequence of SEQ ID NO: 228, 229, 230, 231, or 256 or an RNA equivalent or a DNA/RNA chimeric thereof. In some embodiments, the SARS-CoV-2 target capture oligonucleotide comprises a nucleotide sequence having at least 80%, at least 85% at least 90%, or at least 95% homology to the nucleotide sequence of SEQ ID NO:38, 228, 229, 230, 231, or 256 or an RNA equivalent or a DNA/RNA chimeric thereof. A SARS-CoV-2 target capture oligonucleotide can have 1 or more modified nucleotides or nucleotide analogs.

In some embodiments, a SARS-CoV-2 region 1 target capture oligonucleotide is linked to a TₙAₘ sequence, wherein n in an integer from 0 to 3 and m in an integer from 14 to 50. In some embodiments, n is 3 and m is 30. In some embodiments, an influenza target capture oligonucleotide comprises the nucleotide sequence of SEQ ID NO: 223, 224, 225, 226, or 227 or an RNA equivalent or a DNA/RNA chimeric thereof (Table 3j). In some embodiments, an influenza target capture oligonucleotide consists essentially of the nucleotide sequence of SEQ ID NO: 223, 224, 225, 226, or 227 or an RNA equivalent or a DNA/RNA chimeric thereof. In some embodiments, an influenza target capture oligonucleotide consists of the nucleotide sequence of SEQ ID NO: 223, 224, 225, 226, or 227 or an RNA equivalent or a DNA/RNA chimeric thereof. In some embodiments, an influenza target capture oligonucleotide comprises a nucleotide sequence having no more than 0, 1, 2, 3, 4, or 5 mismatches from the nucleotide sequence of SEQ ID NO: 223, 224, 225, 226, or 227 or an RNA equivalent or a DNA/RNA chimeric thereof. In some embodiments, an influenza target capture oligonucleotide comprises a nucleotide sequence having at least 80%, at least 85% at least 90%, or at least 95% homology to the nucleotide sequence of SEQ ID NO: 223, 224, 225, 226, or 227 or an RNA equivalent or a DNA/RNA chimeric thereof. A SARS-CoV-2 region 1 promoter primer can have 1 or more modified nucleotides or nucleotide analogs.

TABLE 3j
SARS-CoV-2 Target Capture oligo sequences

| SEQ ID NO: | Sequence (5′→3′) |
|---|---|
| 228 | CAAGUAGUGGCACCUUCUUUAGUC |
| 223 | CAAGUAGUGGCACCUUCUUUAGUCTTTAAAAAAAAAAAAAAAAAAAAAAAAAAA |
| | AAA |
| 229 | GGUGGAAUGUGGUAGGAUUACU |
| 224 | GGUGGAAUGUGGUAGGAUUACUTTTAAAAAAAAAAAAAAAAAAAAAAAAAAAA |
| | AA |
| 256 | GGAAAGCAAAACAGAAAGUAGUG |
| 225 | GGAAAGCAAAACAGAAAGUAGUGTTTAAAAAAAAAAAAAAAAAAAAAAAAAAAA |
| | AA |
| 230 | GCCUGCUCAUAAGCUUCUUGAG |
| 226 | GCCUGCUCAUAAGCUUCUUGAGTTTAAAAAAAAAAAAAAAAAAAAAAAAAAAAA |
| | A |
| 231 | CAACAGCCUGCUCAUAAGCUUCUU |
| 227 | CAACAGCCUGCUCAUAAGCUUCUUTAAAAAAAAAAAAAAAAAAAAAAAAAAAAA |
| | A |

In some embodiments, the compositions, formulations, or kits contain one or more positive controls. The positive control can be a nucleic acid containing a target sequence for SARS-CoV-2 region 1, SARS-CoV-2 region 2, SARS-CoV-2 region 3, influenza A region 1, influenza A region 2, influenza B region 1, or influenza B region 2. The positive control can be, but is not limited to DNA. RNA, plasmid, or an in vitro transcript. Exemplary in vitro transcripts are provided in Table 4.

TABLE 4
In vitro transcript sequences used for testing the capture,
amplification and/or detection oligos, compositions, or kits.

| SEQ ID NO: | Sequence (5′→3′) |
|---|---|
| 259 | SARS-CoV-2 Region 1: |
| | GGGCGAAUUGGGUACCGGGCCCCCCCUCGAGGUCGACGGUAUCGAUAAGCUUGA |
| | UAUCGAAUUCCUGCAGCCCGGGGGAUCCAGCUUGGCACUGAUCCUUAUGAAGAU |
| | UUUCAAGAAAACUGGAACACUAAACAUAGCAGUGGUGUUACCCGUGAACUCAUG |
| | CGUGAGCUUAACGGAGGGGCAUACACUCGCUAUGUCGAUAACAACUUCUGUGGC |
| | CCUGAUGGCUACCCUCUUGAGUGCAUUAAAGACCUUCUAGCACGUGCUGGUAAA |
| | GCUUCAUGCACUUUGUCCGAACAACUGGACUUUAUUGACACUAAGAGGGGUGUA |
| | UACUGCUGCCGUGAACAUGAGCAUGAAAUUGCUUGGUACACGGAACGUUCUGAA |
| | AAGAGCUAUGAAUUGCAGACACCUUUUGAAAUUAAAUUGGCAAAGAAAUUUGA |
| | CACCUUCAAUGGGGAAUGUCCAAAUUUUGUAUUUCCCUUAAAUUCCAUAAUCAA |
| | GACUAUUCAACCAAGGGUUGAAAAGAAAAAGCUUGAUGGCUUUAUGGGUAGAA |
| | UUCGAUCUGUCUAUCCAGUUGCGUCACCAAAUGAAUGCAACCAAAUGUGCCUUU |
| | CAACUCUCAUGAAGUGUGAUCAUUGUGGUGAAACUUCAUGGCAGACGGGCGAUU |
| | UUGUUAAAGCCACUUGCGAAUUUUGUGGCACUGAGAAUUUGACUAAAGAAGGU |
| | GCCACUACUUGUGGUUACUUACCCCAAAAUGCUGUUGUUAAAAUUUAUUGUCCA |
| | GCAUGUCACAAUUCAGAAGUAGGACCUGAGCAUAGUCUUGCCGAAUACCAUAAU |
| | GAAUCUGGCUUGAAAACCAUUCUUCGUAAGGGUGGUCGCACUAUUGCCUUUGGA |
| | GGCUGUGUGUUCUCUUAUGUUGGUUGCCAUAACAAGUGUGCCUAUUGGGUUCCA |
| | CGUGCUAGCGCUAACAUAGGUUGUAACCAUACAGGUGUUGUUGGAGAAGGUUCC |
| | GAAGGUCUUAAUGACAACCUUCUUGAAAUACUCCAAAAAGAGAAAGUCAACAUC |
| | AAUAUUGUUGCGGCC |
| 260 | SARS-CoV-2 Region 2: |
| | GGGCGAAUUGGGUACCGGGCCCCCCCUCGAGGUCGACGGUAUCGAUAAGCUUGA |
| | UAUCGAAUUCCUGCAGCCCGGGGGAUCCCAGUAAAACAACUGUAGCGUCACUUA |
| | UCAACACACUUAACGAUCUAAAUGAAACUCUUGUUACAAUGCCACUUGGCUAUG |
| | UAACACAUGGCUUAAAUUUGGAAGAAGCUGCUCGGUAUAUGAGAUCUCUCAAAG |
| | UGCCAGCUACAGUUUCUGUUUCUUCACCUGAUGCUGUUACAGCGUAUAAUGGUU |
| | AUCUUACUUCUUCUUCUAAAACACCUGAAGAACAUUUUAUUGAAACCAUCUCAC |
| | UUGCUGGUUCCUAUAAAGAUUGGUCCUAUUCUGGACAAUCUACACAACUAGGUA |
| | UAGAAUUUCUUAAGAGAGGUGAUAAAAGUGUAUAUUACACUAGUAAUCCUACC |
| | ACAUUCCACCUAGAUGGUGAAGUUAUCACCUUUGACAAUCUUAAGACACUUCUU |
| | UCUUUGAGAGAAGUGAGGACUAUUAAGGUGUUUACAACAGUAGACAACAUUAA |
| | CCUCCACACGCAAGUUGUGGACAUGUCAAUGACAUAUGGACAACAGUUUGGUCC |
| | AACUUAUUUGGAUGGAGCUGAUGUUACUAAAAUAAAACCUCAUAAUUCACAUGA |
| | AGGUAAAACAUUUUAUGUUUUACCUAAUGAUGACACUCUACGUGUUGAGGCUUU |
| | UGAGUACUACCACACAACUGAUCCUAGUUUUCUGGGUAGGUACAUGUCAGCAUU |
| | AAAUCACACUAAAAAGUGGAAAUACCCACAAGUUAAUGGUUUAACUUCUAUUAA |
| | AUGGGCAGAUAACAACUGUUAUCUUGCCACUGCAUUGUUAACACUCCAACAAAU |
| | AGAGUUGAAGUUUAAUCCACCUGCUCUACAAGAUGCUUAUUACAGAGCAAGGGC |
| | UGGUGAAGCUGCUAACUUUUGUGCACUUAUCUUAGCCUACUGUAAUAAGACAGU |
| | AGGUGAGUUAGGUGAUGUUAGAGAAACAAUGAGUUACUUGUUUCAACAUGCCA |
| | AUUUAGAUUCUUGCAAAAGAGUCUUGAACGUGGUGUGUAAAACUUGUGGACAA |
| | CAGCAGACAACCCUUAAGGGUGUAGAAGCUGUUAUGUACAUGGGCACACUUUCU |
| | UAUGAACAAUUUAAGAAAGGUGUUCAGCGGCC |
| 261 | SARS-CoV-2 Region 3: |
| | GGGCGAAUUGGGUACCGGGCCCCCCCUCGAGGUCGACGGUAUCGAUAAGCUUGA |
| | UAUCGAAUUCUUUCACUACUUUCUGUUUUGCUUUCCAUGCAGGGUGCUGUAGAC |
| | AUAAACAAGCUUUGUGAAGAAAUGCUGGACAACAGGGCAACCUUACAAGCUAUA |
| | GCCUCAGAGUUUAGUUCCCUUCCAUCAUAUGCAGCUUUUGCUACUGCUCAAGAA |
| | GCUUAUGAGCAGGCUGUUGCUAAUGGUGAUUCUGAAGUUGUUCUUAAAAAGUU |
| | GAAGAAGUCUUUGAAUGUGGCUAAAUCUGAAUUUGACCGUGAUGCAGCCAUGCA |
| | ACGUAAGUUGGAAAAGAUGGCUGAUCAAGCUAUGACCCAAAUGCGGCC |
| 262 | Influenza A (H1N1): |
| | GGGCGAAUUGGGUACCGGGCCCCCCCUCGAGGUCGACGGUAUCGAUAAGCUUAU |
| | CUGUCUGUGAGACCGAUGCUGUGAAUCAGCAAUCUGUUCACAAGUGGCACACAC |
| | UAGACCAAAAGCAGCUUCUGUGGUCACUGUUCCCAUCCUGUUGUAUAUGAGGCC |
| | CAUGCAACUGGCAAGUGCACCAGUUGAAUAGCUUAGUGACACCUCCUUGGCCCC |
| | AUGGAACGUUAUUUCUCUUUUGAGCUUCUUGUAUAGUUUAACUGCUCUAUCCAU |
| | GUUGUUCGGGUCCCCAUUCCCAUUUAGGGCAUUUUGGACAAAGCGUCUACGCUG |
| | CAGUCCUCGCUCACUGGGCACGGUGAGCGUGAACACAAAUCCUAAAAUUCCCUU |
| | AGUCAGAGGUGACAAGAUUGGUCUUGUCUUUAGCCAUUCCAUGAGAGCCUCAAG |
| | AUCUGUGUUCUUUCCUGCAAAGACACUUUCCAGUCUCUGCGCGAUCUCGGCUUU |
| | GAGGGGGCCUGACGGGAUGAUAGAAAGAACGUACGUUUCGACCUCGGUUAGAAG |
| | ACUCAUGCGGCC |
| 263 | Influenza A (H3N2): |
| | GGGCGAAUUGGGUACCGGGCCCCCCCUCGAGGUCGACGGUAUCGAUAAGCUUAU |
| | CUGCCUAUGAGACCUGUGCUGGGAGUCAGCAAUCUGCUCACAUGUUGCACAUAC |
| | CAGGCCAAAUGCCACUUCAGUGGUUACAGCCCCCAUCCUAUUGUAUAUGAGGCC |
| | CAUGCAACUGGCAAGUGCACCAGCGGAAUAACUGAGAGCUAUUUCUUUGGCCCC |
| | AUGGAACGUUAUCUCUCUCUUAAGUUUCCUAUACAGUUUAACUGCUUUGUCCAU |
| | GUUAUUUGGGUCUCCAUUCCCAUUGAGGGCAUUUUGGACAAAGCGUCUACGCUG |
| | CAGUCCUCGCUCACUGGGCACGGUGAGCGUGAACACAAACCCCAAAAUCCCCUU |
| | AGUCAGAGGUGACAGAAUUGGUCUUGUCUUUAGCCAUUCCAUGAGAGCCUCAAG |
| | AUCUGUGUUUUUCCCAGCAAAGACAUCUUCAAGUCUCUGCGCGAUCUCGGCUUU |
| | GAGGGGGCCUGAUGGAACGAUAGAGAGAACAUACGUUUCGACCUCGGUUAGAAG |
| | GCUCAUGCGGCC |
| 264 | Influenza B (M1): |
| | GGGCGAAUUGGGUACCGGGCCCCCCCUCGAGGUCGACGGUAUCGAUAAGCUUUU |
| | AUAGGUAUUUCUUCACAAGAGCUGAAUUUCCCAUAGAGCUCUGCUUUAGCACUU |
| | CCAUUACAUCCUUUGCAAUUCCUUCCCCAUUCUUCUGACUUGCCCCAAGGGAUC |
| | UCAAUACUCCAAUGUUGCUUUGCAGUUCUUCUGCCAGUUUUUGGACGUCUUCUC |
| | CUUUUCCCAUUCCAUUCAUUGUUUUUGCUGUGUUCAUAGCUGAGACCAUCUGCA |
| | UUUCCCGUCUCACUCCUGGCACUGAGGAUCUCGCUGCUCUGCUAUGAGCUCUGU |
| | GUGAAUGUGAUGCUUGUUUUUCGCACAAAGCACAGAGCGUUCCUAGUUUUACUU |
| | GCAUUGAAUAAUUUCCAGGAUUCAGGUACAUGACCAUGAGACAAUAUAGUAGCG |
| | CUUAGCUUUCAUGGCCUUCUGCUAUUUCAAAUGCUUCAUGAAAGCUCACACAUU |
| | UUCUCAUUUUUCUCUCAGCCAGAGUCAGGCCCUUCUUUUUUGUUGCUGUUGUUC |
| | CCAUUCCCGAUAAGGGCUCUGUGAUGAAUCUUCUUUUUCUUUCCUGGUCUUUGG |
| | GUUUUAAAAAGCAGAUAGAGGCACCAAUUAGUGCUUUCUGUAUAUCAGUUAAGC |
| | AUCUUUUGUUUUUUAUCCAUUCCAAGGCAGAGUCUAGGUCAAAUUCUUUCCCAC |
| | CGAACCAACAGUGUAAUUUUUCUGCUAGUUCUGCUUUGCCUUCUCCAUCUUCUG |
| | UCAAUGAAAGCAGGUAGGCAAUUGUGUCUCCAAACAGCGACAUGCGGCC |
| 265 | Influenza B (NS): |
| | GGGCGAAUUGGGUACCGGGCCCCCCCUCGAGGUCGACGGUAUCGAUAAGCUUCU |
| | AAUUGUCUCCCUCUUCUGGUGAUAAUCGGUGCUCUUGACCAAAUUGGGAUAAGA |
| | CUCCCACCGCAGUUUCAGCUGCUCGAAUUGGCUUUGAAUGUCCUUCAUUAAGAC |
| | GCUCGAAGAGUGAAUUGAGGAUCCGAUGGCCAUCUUCUUCAUCCUCCACUGUAA |
| | GAUCAUCAGUAGCAACAAGUUUAGCAACAAGCCUUCCACUCUGGUCAUAUGCAU |
| | UCAAUCUAUGCAGAGUUGAUAAGGACUUGUAUCCAUUGGGCGGCC |

### IV. Multiplex Amplification Systems

A “multiplex” amplification reaction, such as an isothermal amplification reaction, is characterized in that two or more different amplification products, or amplicons, are generated by means of using two or more sets of amplification primers in the same amplification reaction. A multiplex amplification reaction includes two or more primer sets (e.g., two or more first (NT7) primer and second (promoter) primers sets) for amplifying two target sequences. A multiplex amplification system comprises a composition, formulations, reaction mix, or kit for performing a multiplex amplification and/or detection reaction. In addition to primer sets, a multiplex amplification system can further comprise two or more probes for detecting the corresponding amplicons, and/or two or more TCOs.

In some embodiments, a multiplex amplification system for amplifying and/or detecting the presence or absence of SARS-CoV-2, Influenza A, and Influenza B includes:
(a) at least two primer sets for amplifying target sequences in two or more of SARS-CoV-2, Influenza A, and Influenza B;
(b) least two primer sets for amplifying target sequences in each of SARS-CoV-2 and Influenza A;
(c) at least two primer sets for amplifying target sequences in each of SARS-CoV-2 and Influenza B;
(d) at least two primer sets for amplifying target sequences in each of Influenza A and Influenza B;
(e) at least three primer sets for amplifying target sequences in each of SARS-CoV-2, Influenza A, and Influenza B; or
(f) at least four primer sets for amplifying each of a first target nucleic acid sequence of SARS-CoV-2, a second target nucleic acid sequence of SARS-CoV-2, a target nucleic acid sequence of Influenza A, and a target nucleic acid sequence of Influenza B.

The primer set(s) for amplifying a target sequence in SARS-CoV-2 can be a primer set for amplifying a target sequence of SARS-CoV-2 region 1, SARS-CoV-2 region 2, or SARS-CoV-2 region 3, or a combination thereof. The primer set for amplifying a target sequence in Influenza A can be a primer set for amplifying a target sequence of Influenza A region 1 or Influenza A region 2. The primer set for amplifying a target sequence in Influenza B can be a primer set for amplifying a target sequence of Influenza B region 1 or Influenza B region 2. Nucleic acid sequences for primer sets and probes for amplification and detection of SARS-CoV-2 region 1, SARS-CoV-2 region 2, SARS-CoV-2 region 3, Influenza A region 1, Influenza A region 2, Influenza B region 1, and Influenza B region 2 are described above. Any of the above multiplex amplification systems can further comprise a primer set for amplifying an internal control sequence and optionally a probe for detecting the internal control amplicon, and further optionally a TCO for capturing the internal control sequence.

TABLE 5
Multiplex amplification system combinations. Each of the combinations below may further
include a primer set and optionally a probe and further optionally a TCO for amplifying and
detecting and capturing an internal control target sequence.

| # | Set 1 | Set 2 | Set 3 | Set 4 |
|---|---|---|---|---|
| 1 | SARS-COV-2 region 1 | Influenza A region 1 | | |
| 2 | SARS-COV-2 region 1 | Influenza A region 2 | | |
| 3 | SARS-COV-2 region 2 | Influenza A region 1 | | |
| 4 | SARS-COV-2 region 2 | Influenza A region 2 | | |
| 5 | SARS-COV-2 region 3 | Influenza A region 1 | | |
| 6 | SARS-COV-2 region 3 | Influenza A region 2 | | |
| 7 | SARS-COV-2 region 1 | Influenza B region 1 | | |
| 8 | SARS-COV-2 region 1 | Influenza B region 2 | | |
| 9 | SARS-COV-2 region 2 | Influenza B region 1 | | |
| 10 | SARS-COV-2 region 2 | Influenza B region 2 | | |
| 11 | SARS-COV-2 region 3 | Influenza B region 1 | | |
| 12 | SARS-COV-2 region 3 | Influenza B region 2 | | |
| 13 | SARS-COV-2 region 1 | SARS-COV-2 region 2 | Influenza A region 1 | |
| 14 | SARS-COV-2 region 1 | SARS-COV-2 region 2 | Influenza A region 2 | |
| 15 | SARS-COV-2 region 1 | SARS-COV-2 region 3 | Influenza A region 1 | |
| 16 | SARS-COV-2 region 1 | SARS-COV-2 region 3 | Influenza A region 2 | |
| 17 | SARS-COV-2 region 2 | SARS-COV-2 region 3 | Influenza A region 1 | |
| 18 | SARS-COV-2 region 2 | SARS-COV-2 region 3 | Influenza A region 2 | |
| 19 | SARS-COV-2 region 1 | SARS-COV-2 region 2 | Influenza B region 1 | |
| 20 | SARS-COV-2 region 1 | SARS-COV-2 region 2 | Influenza B region 2 | |
| 21 | SARS-COV-2 region 1 | SARS-COV-2 region 3 | Influenza B region 1 | |
| 22 | SARS-COV-2 region 1 | SARS-COV-2 region 3 | Influenza B region 2 | |
| 23 | SARS-COV-2 region 2 | SARS-COV-2 region 3 | Influenza B region 1 | |
| 24 | SARS-COV-2 region 2 | SARS-COV-2 region 3 | Influenza B region 2 | |
| 25 | Influenza A region 1 | Influenza B region 1 | | |
| 26 | Influenza A region 1 | Influenza B region 2 | | |
| 27 | Influenza A region 2 | Influenza B region 1 | | |
| 28 | Influenza A region 2 | Influenza B region 2 | | |
| 29 | SARS-COV-2 region 1 | SARS-COV-2 region 2 | Influenza A region 1 | Influenza B region 1 |
| 30 | SARS-COV-2 region 1 | SARS-COV-2 region 2 | Influenza A region 1 | Influenza B region 2 |
| 31 | SARS-COV-2 region 1 | SARS-COV-2 region 2 | Influenza A region 2 | Influenza B region 1 |
| 32 | SARS-COV-2 region 1 | SARS-COV-2 region 2 | Influenza A region 2 | Influenza B region 2 |
| 33 | SARS-COV-2 region 1 | SARS-COV-2 region 3 | Influenza A region 1 | Influenza B region 1 |
| 34 | SARS-COV-2 region 1 | SARS-COV-2 region 3 | Influenza A region 1 | Influenza B region 2 |
| 35 | SARS-COV-2 region 1 | SARS-COV-2 region 3 | Influenza A region 2 | Influenza B region 1 |
| 36 | SARS-COV-2 region 1 | SARS-COV-2 region 3 | Influenza A region 2 | Influenza B region 2 |
| 37 | SARS-COV-2 region 2 | SARS-COV-2 region 3 | Influenza A region 1 | Influenza B region 1 |
| 38 | SARS-COV-2 region 2 | SARS-COV-2 region 3 | Influenza A region 1 | Influenza B region 2 |
| 39 | SARS-COV-2 region 2 | SARS-COV-2 region 3 | Influenza A region 2 | Influenza B region 1 |
| 40 | SARS-COV-2 region 2 | SARS-COV-2 region 3 | Influenza A region 2 | Influenza B region 2 |

### V. Detection

A detection step may be performed using any of a variety of known techniques to detect a signal specifically associated with an amplified target sequence, such as, e.g., by hybridizing the amplification product with a labeled detection probe and detecting a signal resulting from the labeled probe (including from label released from the probe following hybridization in some embodiments). In some embodiments, the labeled probe comprises a second moiety, such as a quencher or other moiety that interacts with the first label. Detection may be performed after the amplification reaction is completed or may be performed simultaneously with amplifying the target region, e.g., in real time. In some embodiments, amplified product is detected near or at the end of the amplification step. In some embodiments, a linear detection probe is used to provide a signal to indicate hybridization of the probe to the amplified product. One example of such detection uses a luminescently labeled probe that hybridizes to target nucleic acid. The luminescent label is then hydrolyzed from non-hybridized probe. Detection is performed by chemiluminescence using a luminometer. (See, e.g., International Patent Application Pub. No. WO 89/002476, incorporated by reference herein). In some embodiments, the detection is done in real time. In some embodiments, the detection probe is a hairpin probe. A hairpin probe can be, but is not limited to, a molecular beacon, molecular torch, or hybridization switch probe that is labeled with a reporter moiety that is detected when the probe binds to amplified product (e.g., a dual-labeled hairpin probe comprising both a fluorescent label and a quenching moiety). In some embodiments, the detection probe is a linear oligomer such as, e.g., an oligomer labeled with both a fluorophore and a quenching moiety (e.g., a TaqMan probe). Such probes may comprise target hybridizing sequences and non-target hybridizing sequences. Various forms of such probes have been described previously (see, e.g., U.S. Pat. Nos. 5,210,015; 5,487,972; 5,118,801; 5,312,728; 5,925,517; 6,150,097; 6,849,412; 6,835,542; 6,534,274; and 6,361,945; and US Patent Application Pub. Nos. 20060068417A1 and 20060194240A1; each incorporated by reference herein).

In some embodiments, detection is performed at time intervals. Detection can be done by measuring fluorescence at regular time intervals. Time intervals can be, but are not limited to: 1-60 sec, 1-120 sec, 1-180 sec, 1-240 sec, or 1-300 sec. In some embodiments, the time interval is 5, 10, 15, 20, 25, 30, 35, 40, 45, 50, 55, or 60 sec. For detection performed at regular time intervals, each interval is referred to as a cycle. Detection can be performed for 20-240 cycles, 30-210 cycles, 40-180 cycles, 50-150 cycles, or 60-120 cycles. For example, detection every 30 sec for 60 minutes constitutes 120 cycles. Detection may occur at the beginning or end of a cycle. Detection can also be performed continuously.

Compositions, formulations, and kits for detection of the SARS-CoV-2, Influenza A, and/or Influenza B may include probes that bind to an internal control that is not a SARS-CoV-2, Influenza A, or Influenza B nucleic acid that is amplified and detected in the same assay reaction mixtures or a parallel assay reaction mixture, by using amplification and detection oligomers specific for the IC sequence. IC nucleic acid sequences can be, e.g., a DNA plasmid, an RNA template sequence (e.g., an in vitro transcript), or a synthetic nucleic acid that is spiked into a sample. Alternatively, the IC nucleic acid sequence may be a cellular component, which may be from exogenous cellular sources or endogenous cellular sources relative to the specimen. In these instances, an internal control nucleic acid is co-amplified with the SARS-CoV-2, Influenza A, and/or Influenza B nucleic acids in the amplification reaction mixtures. The internal control amplification product and the SARS-CoV-2, Influenza A, and/or Influenza B target sequence amplification products can be detected independently.

In certain embodiments, amplification and detection of a signal from an amplified IC sequence demonstrates that the assay reagents, equipment, conditions, and performance of assay steps were functioning and used properly in the assay if no signal is obtained for the intended target nucleic acids (e.g., samples that test negative for SARS-CoV-2, Influenza A, and Influenza B). An IC may also be used as an internal calibrator for the assay when a quantitative result is desired, i.e., the signal obtained from the IC amplification and detection is used to set a parameter used in an algorithm for quantitating the amount of target nucleic acid in a sample based on the signal obtained for an amplified target sequence. ICs are also useful for monitoring the integrity of one or more steps in an assay. The primers and probe for the IC target sequence are configured and synthesized by using any well-known method provided that the primers and probe function for amplification of the IC target sequence and detection of the amplified IC sequence using substantially the same assay conditions used to amplify and detect the target sequences. In some embodiments, that include a target capture-based purification step, a target capture probe specific for the IC target is included in the assay in the target capture step so that the IC is treated in the assay in a manner analogous to that for the intended target nucleic acid in all of the assay steps.

The probes utilized in a multiplex amplification system can be labeled such that any one probe species (or probe for detecting a species) can be distinguished from other probe species (or probes for detecting other species) in a multiplex detection assay. In some embodiments, a probe for detecting SARS-CoV-2, region 1, 2, and/or 3, can be distinguished from probes for detecting influenza A, influenza B, and optionally an internal control amplicon. In compositions, reaction mixes or kits for detecting at least two SARS-CoV-2 amplicons, the probes can utilize the same label. In some embodiments, the probe for detecting influenza A, region 1 and/or 2, can be distinguished from probes for detecting SARS-CoV-2, influenza B, and optionally an internal control amplicon. In some embodiments, the probe for detecting influenza B, region 1 and/or 2, can be distinguished from probes for detecting SARS-CoV-2, influenza A. and optionally an internal control amplicon. In some embodiments, the probe for detecting an internal control amplicon, can be distinguished from probes for detecting SARS-CoV-2, influenza A, and influenza B. The labels can be, but are not limited to, fluorophores, and fluorophore/quencher combinations (i.e., FRET hybridization probes as described in Matthews and Kricka, Analytical Biochemistry, vol. 169 (1988), pp: 1-25)).

### VI. Formulations and Kits

Described are formulations and kits for determining the presence or absence of SARS-CoV-2, Influenza A, and/or Influenza B in a sample. In some embodiments, the formulations and kits comprise at least one primer set for amplifying a target sequence in SARS-CoV-2. In some embodiments, the formulations and kits comprise at least one primer set for amplifying a target sequence in Influenza A. In some embodiments, the formulations and kits comprise at least one primer set for amplifying a target sequence in Influenza B. In some embodiments, the formulations and kits comprise at least one primer set for amplifying a target sequence in SARS-CoV-2 and at least one primer set for amplifying a target sequence in Influenza A. In some embodiments, the formulations and kits comprise at least one primer set for amplifying a target sequence in SARS-CoV-2 and at least one primer set for amplifying a target sequence in Influenza B. In some embodiments, the formulations and kits comprise at least one primer set for amplifying a target sequence in Influenza A and at least one primer set for amplifying a target sequence in Influenza B. In some embodiments, the formulations and kits comprise at least one primer set for amplifying a target sequence in SARS-CoV-2, at least one primer set for amplifying a target sequence in Influenza A, and at least one primer set for amplifying a target sequence in Influenza B. In some embodiments, the formulations and kits comprise at least one primer set for amplifying a first target sequence in SARS-CoV-2, at least one primer set for amplifying a second target sequence in SARS-CoV-2, at least one primer set for amplifying a target sequence in Influenza A, and at least one primer set for amplifying a target sequence in Influenza B. In some embodiments, the formulations and kits further comprise an organic buffer.

A primer set can comprise at least one first primer, such as an NT7 primer and at least one second primer, such as a promoter primer. A primer set is used to amplify a target sequence. In some embodiments, a primer set comprises an NT7 primer and a promoter primer. In some embodiments, a primer set comprises an NT7 primer and two different promoter primers. A primer set for amplifying a target sequence in SARS-CoV-2 can be selected from the primer sequences in Tables 3a, 3b, 3d, 3e, 3g, and 3h. A primer set for amplifying a target sequence in Influenza A can be selected from the primer sequences in Tables 1a, 1b, 1d, and 1e. A primer set for amplifying a target sequence in Influenza B can be selected from the primer sequences in Tables 2a, 2b, 2d, and 2e. A kit can contain any of the described NT7 primers and promoter primers described for Influenza A region 1 or 2, Influenza B region 1 or 2, or SARS-CoV-2 regions 1, 2, or 3.

In some embodiments, the formulations and kits further contain one or more probes for detecting the amplified product(s). A kit can include a probe for detection a SARS-CoV-2 amplicon, an influenza A amplicon, an influenza B amplicon, or a combination thereof

In some embodiments, a kit contains one or more of: a Target Capture Reagent (TCR), an amplification (AMP) reagent, and a promoter reagent. A kit may further contain one or more of: buffer, enzyme reagent, DNA polymerase, reverse transcriptase, RNA polymerase, dNTPs, NTPs, Sample Transport Medium, Target Capture Wash Solution, Target Enhancer Reagent, and a reconstitution reagent.

In some embodiments, a biphasic multiplex composition comprises oligonucleotides comprising each of SEQ ID NO: 1, SEQ ID NO: 72, SEQ ID NO: 126, SEQ ID NO: 127, SEQ ID NO: 38, SEQ ID NO: 106, SEQ ID NO: 195 and/or SEQ ID NO: 197, SEQ ID NO: 200, SEQ ID NO: 63 and/or SEQ ID NO: 64, SEQ ID NO: 122, and SEQ ID NO: 247; and at least one probe oligonucleotide having a target hybridizing sequence contained in SEQ ID NO: 135 and comprising SEQ ID NO: 244. In some embodiments, a SARS-CoV-2 Region 1 probe comprising a nucleotide sequence contained in SEQ ID NO: 135 and comprising SEQ ID NO: 244 comprises SEQ ID NO: 244, SEQ ID NO: 234, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, or SEQ ID NO: 275.

In some embodiments, a biphasic multiplex composition comprises: (a) an amplification reagent comprising non-T7 primers comprising SEQ ID NO: 1, SEQ ID NO: 72, SEQ ID NO: 126, and SEQ ID NO: 127; (b) a promoter reagent comprising primers comprising SEQ ID NO: 38, SEQ ID NO: 106, SEQ ID NO: 195 and/or SEQ ID NO: 197, and SEQ ID NO: 200, and probe oligonucleotides comprising SEQ ID NO: 63 and/or SEQ ID NO: 64, SEQ ID NO: 122, and SEQ ID NO: 247, and at least one probe oligonucleotide having a target hybridizing sequence contained in SEQ ID NO: 135 and comprising SEQ ID NO: 244. In some embodiments, a SARS-CoV-2 Region 1 probe comprising a nucleotide sequence contained in SEQ ID NO: 135 and comprising SEQ ID NO: 244 comprises SEQ ID NO: 244, SEQ ID NO: 234, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, or SEQ ID NO: 275.

In some embodiments, a biphasic multiplex composition comprises: (a) a target capture reagent comprising target capture oligonucleotides comprising SEQ ID NO: 51, SEQ ID NO: 111, SEQ ID NO: 228, SEQ ID NO: 229 and primers comprising SEQ ID NO: 38, SEQ ID NO: 106, SEQ ID NO: 195 and/or SEQ ID NO: 197, and SEQ ID NO: 200; (b) an amplification reagent comprising non-T7 primers comprising SEQ ID NO: 1, SEQ ID NO: 72, SEQ ID NO: 126, and SEQ ID NO: 127; (c) and a promoter reagent comprising primers comprising SEQ ID NO: 38, SEQ ID NO: 106, SEQ ID NO: 195 and/or SEQ ID NO: 197, SEQ ID NO: 200, probe oligonucleotides comprising SEQ ID NO: 63 and/or SEQ ID NO: 65, SEQ ID NO: 122, and SEQ ID NO: 247, and at least one probe oligonucleotide having a target hybridizing sequence contained in SEQ ID NO: 135 and comprising SEQ ID NO: 244. In some embodiments, a SARS-CoV-2 Region 1 probe comprising a nucleotide sequence contained in SEQ ID NO: 135 and comprising SEQ ID NO: 244 comprises SEQ ID NO: 244, SEQ ID NO: 234, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, or SEQ ID NO: 275.

Each of the primers of the target capture reagent and/or each of the primers of the promoter reagent can further contain a RNA polymerase promoter sequence, such as a T7 promoter sequence. In some embodiments, SEQ ID NO: 38, SEQ ID NO: 106, SEQ ID NO: 195 and/or SEQ ID NO: 197, and SEQ ID NO: 200 each further contains a RNA polymerase promoter sequence. The RNA polymerase promoter sequence can be, but is not limited to, a T7 RNA polymerase promoter sequence.

Each of the probe oligonucleotides of the promoter reagent can comprise a non-target specific sequence at the 3′ terminus and/or the 5′ terminal that is complementary with another sequence in the probe such that the probe forms an intramolecular double strand region (stem-loop structure) when the probe is not hybridized with a target nucleic acid. In some embodiments, one or more of SEQ ID NO: 63, SEQ ID NO: 64, SEQ ID NO: 122, and SEQ ID NO: 247 further contains a 5′ or 3′ non-target specific sequence 1-7 nucleotides in length.

In some embodiments, a biphasic multiplex composition comprises SEQ ID NO: 167 and/or SEQ ID NO: 169, SEQ ID NO: 172, SEQ ID NO: 235, SEQ ID NO: 28, SEQ ID NO: 55 and/or SEQ ID NO: 54, SEQ ID NO: 98, SEQ ID NO: 116, and at least one probe oligonucleotide having a target hybridizing sequence contained in SEQ ID NO: 135 and comprising SEQ ID NO: 244. The primers of the promoter reagent can further contain a RNA polymerase promoter sequence, such as a T7 promoter sequence. In some embodiments, a SARS-CoV-2 Region 1 probe comprising a nucleotide sequence contained in SEQ ID NO: 135 and comprising SEQ ID NO: 244 comprises SEQ ID NO: 244, SEQ ID NO: 234, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, or SEQ ID NO: 275.

In some embodiments, a biphasic multiplex composition comprises an amplification reagent comprising SEQ ID NO: 1, SEQ ID NO: 72, SEQ ID NO: 126, and SEQ ID NO: 127; and a promoter reagent comprising SEQ ID NO: 28, SEQ ID NO: 98, SEQ ID NO: 167 and/or SEQ ID NO: 169, SEQ ID NO: 172, SEQ ID NO: 54, SEQ ID NO: 55, SEQ ID NO: 116, and SEQ ID NO: 235, and at least one probe oligonucleotide having a target hybridizing sequence contained in SEQ ID NO: 135 and comprising SEQ ID NO: 244. In some embodiments, a SARS-CoV-2 Region 1 probe comprising a nucleotide sequence contained in SEQ ID NO: 135 and comprising SEQ ID NO: 244 comprises SEQ ID NO: 244, SEQ ID NO: 234, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, or SEQ ID NO: 275.

In some embodiments, a biphasic multiplex composition comprises a target capture reagent comprising SEQ ID NO: 48, SEQ ID NO: 108, SEQ ID NO: 223, SEQ ID NO: 224, SEQ ID NO: 28, SEQ ID NO: 98, SEQ ID NO: 167 and/or SEQ ID NO: 169, and SEQ ID NO: 172; an amplification reagent comprising SEQ ID NO: 1, SEQ ID NO: 72, SEQ ID NO: 126, and SEQ ID NO: 127; and a promoter reagent comprising SEQ ID NO: 28, SEQ ID NO: 98, SEQ ID NO: 167 and/or SEQ ID NO: 169, SEQ ID NO: 172 SEQ ID NO: 54, SEQ ID NO: SEQ ID NO: 116, and SEQ ID NO: 235, and at least one probe oligonucleotide having a target hybridizing sequence contained in SEQ ID NO: 135 and comprising SEQ ID NO: 244. In some embodiments, a SARS-CoV-2 Region 1 probe comprising a nucleotide sequence contained in SEQ ID NO: 135 and comprising SEQ ID NO: 244 comprises SEQ ID NO: 244, SEQ ID NO: 234, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, or SEQ ID NO: 275.

Any of the formulations or reagents may be provided as an aqueous solution. An aqueous solution my further comprise a surfactant. Particularly suitable surfactants include, for example, polyethylene glycol mono [4-(1,1,3,3-tetramethylbutyl) phenyl] ether and polyoxyethylene sorbitan fatty acid esters (e.g., polysorbate 20, polysorbate 40, or polysorbate 60). In some embodiments, a surfactant in an aqueous detection probe formulation is a non-linear surfactant (i.e., a surfactant having a branched chain structure) such as, for example, a polyoxyethylene sorbitan fatty acid ester (e.g., polysorbate 20, polysorbate 40, or polysorbate or digitonin. An aqueous formulation as above for amplification or detection of a target nucleic acid may further include a bulking agent such as, e.g., trehalose, raffinose, or a combination thereof. In some embodiments, an aqueous formulation as above contains an inorganic salt such as, e.g., magnesium, potassium, or sodium; in some such variations, the concentration of the inorganic salt is 4 mM or less. A particularly suitable organic buffer for an aqueous formulation as above is Tris (2-amino-2-(hydroxymethyl)-1,3-propanediol). In some embodiments, one or more of formulations or reagents is provided in a dried or lyophilized form.

In a related aspect, for long-term storage, an aqueous formulation as described herein may be dried (e.g., lyophilized). By way of example, the aqueous formulation or a frozen version of the aqueous formulation may be aliquoted into, e.g., vials, ampules, or other containers, and dried (e.g., lyophilized) according to procedures known in the art. The dried product typically appears as a powder or a cake or a sphere. The containers are then sealed. In some embodiments, the dried formulation aliquots are transferred to wells of a multi-well plate and the multi-well plate is then sealed. Methods of preparing such dried formulations from the aqueous formulation, as well as the dried formulations prepared by such methods, are additional aspects of the instant disclosure. In some embodiments, there is provided a dried formulation that enables reconstitution into an aqueous formulation as described herein. Dried formulations for amplification or detection of a target nucleic acid typically contain, in addition to one or more amplification oligomers and/or detection probes as described herein, a bulking agent such as, e.g., trehalose, raffinose, or a combination thereof. In some embodiments further comprising an inorganic salt, the percent mass of the inorganic salt to the mass of the dried formulation is 0.249% or less, 0.222% or less, or 0.195% or less. Methods of preparing a dried formulation from a lyophilized formulation as described herein are also encompassed by the instant disclosure; such methods generally include dissolving the dried formulation in a suitable diluent (e.g., an organic buffer or water) to provide a reconstituted formulation.

In some embodiments, reaction mixture in accordance with the present disclosure includes one or both of (1) one or more primer sets as described herein for amplification of SARS-CoV-2, Influenza A, and/or Influenza B target sequences and (2) one or more probe as described herein for determining the presence or absence of a SARS-CoV-2; Influenza A, and/or Influenza B amplification product. The reaction mixture may further include several optional components such as, for example, a TCO. For an amplification reaction mixture, the reaction mixture will typically include other reagents suitable for performing in vitro amplification such as, e.g., buffers, salt solutions, appropriate nucleotide triphosphates (e.g., dATP, dCTP, dGTP, and dTTP; and/or ATP, CTP, GTP and UTP), and/or enzymes (e.g., a thermostable DNA polymerase, or reverse transcriptase and/or RNA polymerase), and will typically include test sample components. In addition, for a reaction mixture that includes a detection probe together with a primer set, selection of primers and probe for a reaction mixture are linked by a common target region (i.e., the reaction mixture will include a probe that binds to a sequence amplifiable by primer set of the reaction mixture). In some embodiments, a reaction mixture comprises an aqueous formulation as described above. In some embodiments, a reaction mixture is reconstituted with water, a reconstitution reagent, or an organic buffer from a dried formulation as described above. In some embodiments, a reaction mixture comprises a TCO and a primer. In some embodiments, a reaction mixture comprises a primer. In some embodiments, a reaction mixture comprises a primer set. In some embodiments, a reaction mixture comprises a probe. In some embodiments, a reaction mixture comprises a primer and a probe. In some embodiments, a reaction mixture comprises a primer set and a probe. In some embodiments, the reaction mixture comprises a TCO, a primer set, and a probe.

Also provided herein are kits for practicing the methods as described herein. A kit in accordance with the present disclosure includes one or both of (1) one or more primer sets as described herein for amplification of SARS-CoV-2, Influenza A, and/or Influenza B target sequences and (2) one or more probe as described herein for determining the presence or absence of a SARS-CoV-2, Influenza A, and/or Influenza B amplification product. The kits may further include several optional components such as, for example, a TCO. Other reagents that may be present in the kits include reagents suitable for performing in vitro amplification such as, e.g., buffers, salt solutions, appropriate nucleotide triphosphates (e.g., dATP, dCTP, dGTP, dTTP; and/or ATP, CTP, GTP and UTP), and/or enzymes (e.g., a thermostable DNA polymerase, or a reverse transcriptase and/or RNA polymerase). Oligomers as described herein may be packaged in a variety of different embodiments, and those skilled in the art will appreciate that the disclosure embraces many different kit configurations. For example, a kit may include primers sets for only one target nucleic acid, or it may include amplification oligomers for multiple target regions. In addition, for a kit that includes a probe together with a primer set, selection of primers and probes for a kit are linked by a common target region (i.e., the kit will include a probe that binds to a sequence amplifiable by a primer set of the kit). In certain embodiments, the kit further includes a set of instructions for practicing methods in accordance with the present disclosure, where the instructions may be associated with a package insert and/or the packaging of the kit or the components thereof.

Any method disclosed herein is also to be understood as a disclosure of corresponding uses of materials involved in the method directed to the purpose of the method. Any of the oligonucleotide and any combinations (e.g., kits and compositions) comprising such an oligonucleotides are to be understood as also disclosed for use in amplifying and/or detecting SARS-CoV-2, Influenza A, and/or Influenza B, and for use in the preparation of a composition for amplifying and/or detecting SARS-CoV-2, Influenza A, and/or Influenza B.

The compositions, kits, formulations, reaction mixtures, and methods are further illustrated by the following non-limiting examples.

### EXAMPLES

### Example 1. Real-Time Amplification and Detection of Virus Using Different

Combinations of Primers and Probe. This example describes screening experiments testing primer and probe combinations for the real-time amplification and detection of coronavirus. Reactions are generally prepared and performed as presented herein and as follows.

Several primer and probe mixtures (PPR mixes) are prepared in a microfuge tube or other suitable receptable or container to include a forward primer, a reverse primer, and a dual labeled hydrolysis detection probe. The internal control PPR mix comprises 0.625 μM of each primer and 0.5 μM of the probe, while the virus PPR mixes contain 1.25 μM of each of the primers and the probe. These PPR mixes also contain 150 mM of KCl, 10 mM MgCl₂, and are brought to final volume using 10 mM TRIS. The internal control (IC) detection probe is labeled with Quasar 705 and Black Hole Quencher 2 and each virus detection probe is labeled with FAM and Black Hole Quencher 1 (all available from BioSearch Technologies, Inc., Novato, CA or Glen Research, Inc., Sterling, VA).

An equal volume of the internal control PPR mix (275 μL) is added each of the virus mixtures (275 μL) to provide 1.25×PPR mixes (550 μL total volume). Each of the 1.25×PPR mixes is then overlaid with 250 μL of oil. Synthetic virus target nucleic acids (in vitro test sequences) are prepared from a stock concentration to provide 1,000 copies per reaction in a 5 μL aliquot (200 copies/μL). Dilutions are made into a sample transport media (containing lithium lauryl sulfate (LLS), EDTA, and sodium phosphate). Amplification and detection reactions are set up at 12 reactions per condition; 6 reactions positive for the target nucleic acid and 6 reactions negative for the target nucleic acid. Negative reactions include sample transport media without the virus target nucleic acid.

The resulting amplification curves are evaluated for differences in Ct and RFU signal for the positive samples, and background RFU for the negative samples.

### Example 2. Multi-Phase (BiPhasic) Amplification/Detection

“Sample Transport Medium” or “STM” is a phosphate-buffered solution (pH 6.7) that included EDTA, EGTA, and lithium lauryl sulfate (LLS).

“Target Capture Reagent” or “TCR” is a HEPES-buffered solution (pH 6.4) that includes lithium chloride and EDTA, together with 125 mg/ml of magnetic particles (1 micron SERA-MAG™ MG-CM particles, Seradyn, Inc. Indianapolis, IN) with (dT)14 oligonucleotides covalently bound thereto. TCR contains multiple oligos that may include one or more TCOs and one or more promoter primers. For multiplex amplification, a TCR contains a TCO and a T7 promoter primer configured to hybridize to each target nucleic acid to be amplified and/or detected. In some embodiments, the target nucleic acids to be amplified and/or detected are selected from the group consisting of: SARS-CoV-2 regions 1, 2, and 3, Influenza A regions 1 and 2, and Influenza B regions 1 and 2.

“Amplification Reagent,” “AMP Reagent,” or “AR” is a Tris-buffered solution (pH 7-8, pH 7.5+5, or pH 7.0, pH 7.1, pH 7.2, pH 7.3, pH 7.4, pH7.5, pH 7.6, pH 7.7, pH 7.8, pH 7.9, or pH 8) that includes magnesium chloride, potassium chloride, four deoxyribonucleotide triphosphates (dATP, dCTP, dGTP, and dTTP), four ribonucleotide triphosphates (NTPs: ATP, CTP, GTP, and UTP) and one or more non-promoter (NT7) primers. For multiplex amplification, an AMP Reagent contains a non-promoter primer configured to hybridize to each target nucleic acid to be amplified and/or detected. In some embodiments, the target nucleic acids to be amplified and/or detected are selected from the group consisting of: SARS-CoV-2 regions 1, 2, and 3, Influenza A regions 1 and 2, and Influenza B regions 1 and 2. In some embodiments, AMP Reagent further comprises reverse transcriptase, RNA polymerase, salts and cofactors. The reverse transcriptase can be, but it not limited to MMLV reverse transcriptase (RT). The RNA polymerase can be, but is not limited to, T7 RNA polymerase.

“Promoter Reagent” or “PR” is a Tris buffered solution that includes magnesium chloride, potassium chloride, four deoxyribonucleotide triphosphates (dATP, dCTP, dGTP, and dTTP), four ribonucleotide triphosphates (NTPs: ATP, CTP, GTP, and UTP), one or more promoter primers, and one or more probes. The promoter primer in the Promoter Reagent targets the same target nucleic acid as the promoter primer in the TCR. The promoter primer may have the same sequence as the promoter primer in the TCR or it may have a sequence that is different from the promoter primer in the TCR. For multiplex amplification, a Promoter Reagent contains a promoter primer configured to hybridize to each target nucleic acid to be amplified and/or detected. In some embodiments, the target nucleic acids to be amplified and/or detected are selected from the group consisting of: SARS-CoV-2 regions 1, 2, and 3, Influenza A regions 1 and 2, and Influenza B regions 1 and 2. In some embodiments, for multiplex amplification, the Promoter Reagent contains a probe configured to hybridize to each target nucleic acid to be amplified and/or detected. In some embodiments, the target nucleic acids to be amplified and/or detected are selected from the group consisting of: SARS-CoV-2 regions 1, 2, and 3, Influenza A regions 1 and 2, and Influenza B regions 1 and 2. In some embodiments, Promoter Reagent further comprises reverse transcriptase, RNA polymerase, salts and cofactors. The reverse transcriptase can be, but it not limited to MMLV reverse transcriptase (RT). The RNA polymerase can be, but is not limited to, T7 RNA polymerase.

In some embodiments, one or more of the TCR, AR and PR are lyophilized reagents, each of which is reconstituted just prior to use.

“Target Capture Wash Solution” or “TC Wash Solution” is a HEPES-buffered solution (pH 7-8, pH 7.5±5, or pH 7.5) that included sodium chloride, EDTA, 0.3% (v/v) absolute ethanol, 0.02% (w/v) methyl paraben, 0.01% (w/v) propyl paraben, and 0.1% (w/v) sodium lauryl sulfate.

“Enzyme Reagent”, as used in amplification or pre-amplification reaction mixtures, are HEPES-buffered solutions (pH 6.5-8, pH 7.015, or pH 6.5, pH 6.6, pH 6.7, pH 6.8, pH 6.9, pH 7.0, pH 7.1, pH 7.2, pH 7.3, pH 7.4, pH7.5, pH 76, pH 7.7, pH 7.9, or pH 8) that include MMLV reverse transcriptase (RT), T7 RNA polymerase, salts and cofactors.

A T7 (promoter) primer and TCO are hybridized to the target sequence during target capture, followed by removal of excess T7 primer during a wash step prior to a first amplification reaction. In some embodiments, a TCO is hybridized to the target sequence during target capture. Excess TCO may also be removed during a wash step prior to a first amplification reaction.

During the first amplification phase, AMP reagent, and optionally Enzyme Reagent, is introduced. In the presence of reverse transcriptase, the T7 primer hybridized to the captured target is extended, creating a cDNA copy. The NT7 primer subsequently hybridizes to the cDNA and is extended, filling in the promoter region of the T7 primer and creating an active, double-stranded DNA template. T7 polymerase then produces multiple RNA transcripts from the template. The NT7 primer subsequently hybridized to the RNA transcripts and is extended, producing promoterless cDNA copies of the target RNA template. The RNA strands are degraded by RNase activity of the reverse transcriptase. Because no free T7 primer is available in the phase 1 amplification mixture, the reaction does not proceed further. The second phase is started with the addition of Promoter Reagent, thus initiating exponential amplification and detection of the cDNA pool produced in phase 1.

For multiplex amplification and detection, a TCO, one or two T7 primers, an NT7 primer, and a probe for amplification and detection of each target nucleic acid in the sample is used. The oligonucleotides may amplify one or more different sequence in the same target nucleic acid, may amplify sequences in different target nucleic acids, or a combination thereof. The different target nucleic acids may be from the same or different organisms.

Plate Setup: In some embodiments, four different plates are set up for use on two automated KingFisher devices. The reactions can also be carried out in tubes or other containers.
1. Plate 1 (TCR plate) contains the sample. Target Capture Reagent (e.g., 100 μL) is added to this plate. The TCO and T7 primer hybridize to target nucleic acid (e.g., 400 μL sample). The TCO:target nucleic acid:T7 primer (pre-amplification hybrid) are captured using magnetic beads (capture probe on solid support) using a magnet. For single phase TMA, T7 primer may be absent from the TCR mixture. In some embodiments, sample is combined with TCR containing TCO and, for biphasic amplification, T7 primer. In some embodiments, the mixture of sample and TCR is incubated at elevated temperature for a time duration to facilitate TCO and T7 primer hybridization to the target nucleic acid. The TCO is also hybridized to the magnetic beads. The target nucleic acid with the hybridized TCO and T7 primer (pre-amplification hybrid) is captured by using a magnet to separate the magnetic beads. The mixture of sample and TCR is then removed from the tube and beads are washed twice with Aptima wash buffer.
2. Plate 2 is a deep-well plate and holds 200-500 APTIMA wash buffer. The Aptima wash buffer contains detergent and alcohol used to wash the captured pre-amplification hybrid.
3. Plate 3 contains 200-500 APTIMA wash buffer and is used to provide a second wash of the captured pre-amplification hybrid.
4. Plate 4 contains 50 μL/well AMP reagent. In some embodiments, the AMP reagent contains buffer, salt, dNTPs, NTPs and one or more NT7 primers.

Target Capture and isolation: For BiPhasic TMA, TCO(s) and T7 primer(s) are added to a sample containing or suspected of containing the target nucleic acid. TCO, and T7 primer are incubated with the target nucleic acid for a period of time to allow hybridization of these oligomers to the target nucleic acid to form a pre-amplification hybrid. The pre-amplification hybrid is isolated (captured) using magnetic particles having a binding partner, such as a poly(dT), and purified (washed) to remove excess or non-hybridized oligomers.
1. Plate 1 (TCR plate) is placed into a heat block and heated to 60-65° C. (e.g., 62° C.) for 20-30 min. followed by incubation at lower temperatures (e.g., 23° C.) for 20 min-2 h. In some embodiments, the TCR plate is covered with a 65° C. lid to prevent condensation from forming on the tops of the wells. The captured pre-amplification hybrid is then transferred to Plate 2.
2. After the first wash (about 10 min), a deep well comb/magnet cover is added to the Plate 2 to capture the pre-amplification hybrid. The captured pre-amplification hybrid is transferred to Plate 3.
3. After an optional second wash, a small comb (magnet cover) is added to Plate 3 to capture the pre-amplification hybrid. The washed pre-amplification hybrid is captured and transferred to Plate 4. The 4th plate is processed for real-time isothermal amplification and detection.

BiPhasic Transcription Mediated Amplification and Real Time detection.

First Phase Amplification: AMP Reagent (e.g., 50 μL) containing NT7 primer(s), enzymes, dNTPs, and NTPs, is added to the purified the pre-amplification hybrids. The mixture is incubated for a period of time to allow formation of a first amplification product.
1. Incubate AMP plate containing NT7 primer and purified pre-amplification hybrids, at about 42-44° C. for 5-15 minutes (e.g., 43° C. for 5 minutes).
2. Add 25 μL of Enzyme reagent containing Reverse transcriptase and T7 RNA polymerase, seal, and mix (e.g., 1400 RPMs for 1 minute); incubate about 5 minutes at about 42-44° C. to generate a first amplification product.

Second Phase Amplification: Promoter Reagent containing T7 primer and probe, such as a Torch, is added to the first amplification product and incubated for a period of time to allow formation of a second amplification product. In some embodiments, one or more NT7 primers are added during the second phase amplification.
3. Add 25 μL Promoter Reagent to each well, seal, and mix (e.g., 1400 RPMs for 1 minute). In some embodiments, the Promoter Reagent contains buffer, salt, surfactant, dNTPs, NTPs, one or more T7 primers, Torch probe(s) and optionally more NT7 primers.
4. Run reaction program: incubate at 42-43° C. for 30-60 min (e.g., 120 cycles of 30 seconds) with label detection (collection) at the end of each cycle.

Detection: Amplification of the target nucleic acid sequence is detected in real time by recording fluorescent signal from the probe at regular intervals.

The following examples were performed generally as described herein and exemplified in examples 1 and/or 2.

### Example 3. Singleplex CoV Amplification and Detection Oligonucleotide Combinations

The purpose of this experiment was to test the performance of several candidate torch oligonucleotides and T7 promoter primers in a biphasic, real-time, TMA assay. Testing was done as singleplex reactions for each condition. The following reaction conditions were prepared.

A series of target capture reagents were prepared to contain a target capture oligonucleotide (SEQ ID NO: 223) and one of two T7 promoter primers (SEQ ID NOs: 168 and 169). These target capture reagents were each then split into three to accommodate separately testing along with 3 different promoter reagents, each containing a different torch oligonucleotide. Each of these three promoter reagents contained a T7 promoter primer (SEQ ID NO: 167) and one of three different torch oligonucleotides (SEQ ID NOs: 232, 233 and 234). An amplification reagent was also prepared to contain a non-T7 primer (SEQ ID NO: 126). These reagents were prepared, and the reactions were performed as is generally described above in Example 2.

Each of the different reaction conditions were run in replicates of five (5) for wells containing sample transport media alone (negative control wells) or containing a target nucleic acid at various concentrations. The target nucleic acid for this experiment was an in vitro transcript (SEQ ID NO: 259) at 30 copies, 100 copies, 300 copies or 15,000 copies per mL. Results are presented below.

TABLE 3-1

| | | TCR | | | | | | |
|---|---|---|---|---|---|---|---|---|
| | | SEQ ID NOs: 223 and 169 | | | SEQ ID NOs: 223 and 168 | | | |
| | | Amp Reagent | | | | | | |
| | | SEQ ID NO 126 | | | | | | |
| | | Promoter Reagent | | | | | | |
| | | SEQ ID | SEQ ID | SEQ ID | SEQ ID | SEQ ID | SEQ ID | |
| | | NOs: 167 | NOs: 167 | NOs: 167 | NOs: 167 | NOs: 167 | NOs: 167 | |
| | | and 232 | and 233 | and 234 | and 232 | and 233 | and 234 | |
| 0 | copies/mL | 100% (5/5) | 100% (5/5) | 20% (1/5) | 100% (5/5) | 100% (5/5) | 20% | (1/5) |
| 30 | copies/mL | 100% (5/5) | 100% (5/5) | 100% (5/5) | 100% (5/5) | 100% (5/5) | 100% | (5/5) |
| 100 | copies/mL | 100% (5/5) | 100% (5/5) | 100% (5/5) | 100% (5/5) | 100% (5/5) | 0% | (0/5) |
| | | 100% (5/5) | | | | | | |
| 300 | copies/mL | 100% (5/5) | 100% (5/5) | 100% (5/5) | 100% (5/5) | 100% (5/5) | 100% | (5/5) |
| 15,000 | copies/mL | 100% (5/5) | 100% (5/5) | 100% (5/5) | 100% (5/5) | 100% (5/5) | 100% | (5/5) |

These conditions showed positive signals in from 1 to 5 replicates of each of the negative control wells. False positive results are possibly due to the torch molecules exhibiting intramolecular and/or intermolecular interactions. Despite these false positive results, the conditions containing molecular torch SEQ ID NO: 234 showed low background to signal in the positive wells (negative reaction well RFU from about 0 to 1500, compared to positive reaction well RFU from about 7,500 to 11,000), thus out-performing combinations containing the other two torches. The T7 promoter primers in both target capture reagents (SEQ ID NOs: 168 and 169) performed equally well

A second experiment was performed to test additional torch and T7 promoter primer combinations. Testing was again done as singleplex reactions for each condition. In this second experiment, the target capture reagent contained SEQ ID NO: 224 and one of SEQ ID NO: 180 or SEQ ID NO: 181; the amplification reagent contained SEQ ID NO: 127; and the promoter reagent contained SEQ ID NO: 179 and one of SEQ ID NO: 237, SEQ ID NO: 238, SEQ ID NO: 239, or SEQ ID NO: 240.

Each of the different reaction conditions were run in replicates of five (5) for wells containing sample transport media alone (negative control wells) or containing a target nucleic acid at various concentrations. The target nucleic acid for this experiment was an in vitro transcript (SEQ ID NO: 260) at 30 copies, 100 copies, or 300 copies or 15,000 copies per mL. Results are presented below.

TABLE 3-2

| | | TCR | | | | | | | | | | | | | | | |
|---|---|---|---|---|---|---|---|---|---|---|---|---|---|---|---|---|---|
| | | SEQ ID NOs: 224 and 181 | | | | | | | | SEQ ID NOs: 224 and 180 | | | | | | | |
| | | Amp Reagent | | | | | | | | | | | | | | | |
| | | SEQ ID NO: 127 | | | | | | | | | | | | | | | |
| | | Promoter Reagent* | | | | | | | | | | | | | | | |
| | | 179 and | | 179 and | | 179 and | | 179 and | | 179 and | | 179 and | | 179 and | | 179 and | |
| | | 238 | | 239 | | 240 | | 237 | | 238 | | 239 | | 240 | | 237 | |
| 0 | copies/mL | 0% | (0/5) | 0% | (0/5) | 0% | (0/5) | 0% | (0/5) | 0% | (0/5) | 0% | (0/5) | 0% | (0/5) | 0% | (0/5) |
| 30 | copies/mL | 80% | (4/5) | 100% | (5/5) | 80% | (4/5) | 100% | (5/5) | 100% | (5/5) | 100% | (5/5) | 80% | (4/5) | 80% | (4/5) |
| 100 | copies/mL | 100% | (5/5) | 100% | (5/5) | 100% | (5/5) | 80% | (4/5) | 100% | (5/5) | 100% | (5/5) | 80% | (4/5) | 80% | (4/5) |
| 300 | copies/mL | 100% | (5/5) | 100% | (5/5) | 100% | (5/5) | 60% | (3/5) | 100% | (5/5) | 100% | (5/5) | 100% | (5/5) | 100% | (5/5) |

| | | | | | | | | | | | | | |
|---|---|---|---|---|---|---|---|---|---|---|---|---|---|
| 15,000 | copies/mL | Not tested | Not tested | Not tested | Not tested | 100% | (5/5) | 100% | (5/5) | 100% | (5/5) | 100% | (5/5) |

*Numbers in the Promoter Reagent row are SEQ ID NOs.

These conditions showed no false positive signals. Conditions containing either promoter primer in the target capture reagent and the molecular torch SEQ ID NO: 239 performed well, as did the condition containing promoter primer SEQ ID NO: 180 in the target capture reagent and torch SEQ ID NO: 238. The conditions containing torch SEQ ID NO: 239, though, showed fast TTimes (13 minutes to 21 minutes) and high RFU values (1,000 to 4,000) over a low background. Furthermore, conditions containing torch SEQ ID NO: 239 showed a low spread of these TTime and RFU values between the replicates and across the target concentrations.

### Example 4. SARS-CoV-2 Multiplex Assay

An experiment was performed to amplify and detect SARS-CoV-2 target nucleic acids in a multiplex reaction configuration wherein two separate target sequences of the SARS-CoV-2 target nucleic acid are each detected. The two target sequences were represented in the reactions using in vitro transcripts (“IVTs” SEQ ID NOs: 259 and 260). The multiplex target capture reagent contained SEQ ID NOs: 223 and 224 as target capture oligomers and SEQ ID NOs: 169 and 181 as T7 promoter primers. The multiplex amplification reagent contained SEQ ID NOs: 126 and 131 as non-T7 primers. The promoter reagent contained SEQ ID NOs: 167 and 181 as T7 promoter primers and SEQ ID NOs: 232 and 237 as molecular torch oligomers. SEQ ID NO: 232 was labeled with Fam/Dabcyl and SEQ ID NO: 237 was labeled with ROX/BHQ-2. Reactions were run in replicates of three, the replicates contained either 30, 100, or 300 copies/mL of the SEQ ID NO: 259 IVT; 30, 100, or 300 copies/mL of the SEQ ID NO: 260 WT; or 30, 100, or 300 copies/mL of both the SEQ ID NOs: 259 and 260 IVTs. The reactions were set up and run as is generally described above for multiplex, biphasic, real-time TMA reactions.

Results (shown below) in the FAM channel showed fast average TTimes and strong average fluorescent signal (background cutoff was 1,000 RFU). However, the FAM channel system showed false positives in reaction wells containing the non-target nucleic acid and in the negative control reaction wells, suggesting an intermolecular and/or intramolecular interaction with SEQ ID NO: 232 in this system. Results in the ROX channel showed a slower average TTimes than was seen for the FAM channel system. Also, the ROX channel results showed a weaker average fluorescent signal between 3,300 and 4,100.

TABLE 4-1

| | | R1 | R2 |
|---|---|---|---|
| | | (SEQ | (SEQ |
| | | ID | ID |
| reagent | oligo | NOS) | NOs) |
| TCR | TCR | 223 | 224 |
| | Promoter primer | 169 | 181 |
| Amp Reagent | Non-T7 primer | 126 | 131 |
| Promoter Reagent | Promoter primer | 167 | 181 |
| | torch | 232 | 237 |

TABLE 4-2

| | FAM Channel | FAM Channel | ROX Channel | ROX Channel |
|---|---|---|---|---|
| Target | Avg TTime | Avg RFU | Avg TTime | Avg RFU |
| 30 copies/mL | 13.9 minutes | 12,434 RFU | neg | neg |
| SEQ ID NO: R1 | | | | |
| 100 copies/mL | 13.3 minutes | 12,489 RFU | neg | neg |
| SEQ ID NO: R1 | | | | |
| 300 copies/mL | 12.6 minutes | 11,647 RFU | neg | neg |
| SEQ ID NO: R1 | | | | |
| 30 copies/mL | 14.5 minutes | 13,779 RFU | 19.1 minutes | 3,763 RFU |
| SEQ ID NO: R2 | | | | |
| 100 copies/mL | 14.6 minutes | 13,349 RFU | 17.9 minutes | 4,025 RFU |
| SEQ ID NO: R2 | | | | |
| 300 copies/mL | 14.9 minutes | 13,032 RFU | 16.8 minutes | 3,493 RFU |
| SEQ ID NO: R2 | | | | |
| 30 copies/mL | 14.1 minutes | 13,079 RFU | 18.7 minutes | 3,756 RFU |
| SEQ ID NOs: R1 + R2 | | | | |
| 100 copies/mL | 13.6 minutes | 12,625 RFU | 17.5 minutes | 3,701 RFU |
| SEQ ID NOs: R1 + R2 | | | | |
| 300 copies/mL | 13.1 minutes | 11,961 RFU | 16.5 minutes | 3,357 RFU |
| SEQ ID NOs: R1 + R2 | | | | |
| Neg Control | 14.6 minutes | 12,410 RFU | neg | neg |

### Example 5. Multiplex Assays Having Varied Torches, Non-T7 Primers and T7 Promoter Primers for Detecting SARS-CoV-2

A series of multiplex amplification and detection reactions were prepared, each of the reactions containing a different T7 promoter primer and a different Non-T7 primer. These multiplex reactions each amplify and detect different target sequences within a SARS-CoV-2 target nucleic acid. Target sequences of the SARS-CoV-2 target nucleic acids were represented by SEQ ID NOs: 259 and 260. The reactions were performed in replicates of three and containing 30 copies/mL of SEQ ID NO: 259, 30 copies/mL or 300 copies/mL of SEQ ID NO: 260, or sample transport media alone as a negative control. The following multiplex reaction conditions were prepared. The target capture reagent contained SEQ ID NOs: 223 and 224 as target capture oligonucleotides and contained SEQ ID NO: 169 combined with one of SEQ ID NOs: 170 and 172 as T7 promoter primers. The amplification reagent contained SEQ ID NO: 126 and one of SEQ ID NOs: 129 and 130 as non-T7 primers. The promoter reagent contained SEQ ID NO: 232 combined with one of SEQ ID NOs: 236 and 237 as torch oligonucleotides and SEQ ID NO: 167 combined with one of SEQ ID NOs: 170 and 172 as T7 promoter primers. The reactions were set up and run as is generally described above for multiplex, biphasic, real-time TMA reactions. Background signal cutoff was 1,000 RFU.

As shown in the results below, the FAM channel showed higher average RFU than was seen in the ROX channel (about 7,000 to 13,000 RFU compared to about 3,000 to 4,200 RFU). Average TTimes were also faster for the FAM channel system compared to the ROX channel system. Both systems showed weak false positive signals, suggesting intermolecular and/or intramolecular interactions occurring with the torch oligonucleotides. In this example, the condition containing SEQ ID NOs: 126, 130, 167, 169, 172, 223, 224, 232, and 237 showed the best performance and lowest background signal.

TABLE 5-1

| | FAM Channel | FAM Channel | ROX Channel | ROX Channel |
|---|---|---|---|---|
| Conditions | Avg TTime (min) | Avg RFU | Avg TTime (min) | Avg RFU |
| SEQ ID NOs: | 0 c/mL = 24.4 | 0 c/mL = 1,348 | 0 c/mL = 0 | 0 c/mL = 931 |
| 126, 130, 167, | 30 c/mL = 11.3 | 30 c/mL = 13,179 | 30 c/mL = 0 | 30 c/mL = 839 |
| 169, 172, 223, | 30 c/mL = 19.5 | 30 c/mL = 1,037 | 30 c/mL = 16.4 | 30 c/mL = 3,999 |
| 224, 232, 236 | 300 c/mL = 20.5 | 300 c/mL = 1,036 | 300 c/mL = 14.0 | 300 c/mL = 3,859 |
| SEQ ID NOS: | 0 c/mL = 18.1 | 0 c/mL = 2,214 | 0 c/mL = 0 | 0 c/mL = 896 |
| 126, 130, 167, | 30 c/mL = 11.2 | 30 c/mL = 12,016 | 30 c/mL = 0 | 30 c/mL = 798 |
| 169, 170, 223, | 30 c/mL = 19.6 | 30 c/mL = 1,285 | 30 c/mL = 15.4 | 30 c/mL = 4,464 |
| 224, 232, 236 | 300 c/mL = 16.2 | 300 c/mL = 1,380 | 300 c/mL = 13.3 | 300 c/mL = 3,788 |
| SEQ ID NOS: | 0 c/mL = 0 | 0 c/mL = 321 | 0 c/mL = 0 | 0 c/mL = 1,385 |
| 126, 129, 167, | 30 c/mL = 10.9 | 30 c/mL = 7,347 | 30 c/mL = 0 | 30 c/mL = 525 |
| 169, 172, 223, | 30 c/mL = 0 | 30 c/mL = 116 | 30 c/mL = 17.6 | 30 c/mL = 3,010 |
| 224, 232, 236 | 300 c/mL = 0 | 300 c/mL = 226 | 300 c/mL = 14.7 | 300 c/mL = 4.308 |
| SEQ ID NOs: | 0 c/mL = 0 | 0 c/mL = 242 | 0 c/mL = 0 | 0 c/mL = 1,373 |
| 126, 130, 167, | 30 c/mL = 11.0 | 30 c/mL = 12,362 | 30 c/mL = 0 | 30 c/mL = 1,362 |
| 169, 172, 223, | 30 c/mL = 0 | 30 c/mL = 157 | 30 c/mL = 16.4 | 30 c/mL = 3,820 |
| 224, 232, 237 | 300 c/mL = 0 | 300 c/mL = 125 | 300 c/mL = 13.8 | 300 c/mL = 4,281 |

TTime and RFU Data cells recite, in descending order sample transport media, 30 copies/mL of SEQ ID NO: 259, 30 copies/mL of SEQ ID NO: 260 and 300 copies/mL of SEQ ID NO: 260.

### Example 6. Multiplex Assays Having Varied Torch Oligonucleotide Components

A series of multiplex amplification and detection reactions were prepared, each of the reactions containing a different torch oligonucleotide. These multiplex reactions each amplify and detect different target sequences within a SARS-CoV-2 target nucleic acid. The following multiplex reaction conditions were prepared. The target capture reagent contained SEQ ID NOs: 223 and 224 as target capture oligonucleotides and SEQ ID NOs: 169 and 172 as T7 promoter primers. The amplification reagent contained SEQ ID NOs: 126 and 127 as non-T7 primers. The promoter reagent contained SEQ ID NOs: 167 and 172 as T7 promoter primers and SEQ ID NO: 232 combined with one of SEQ ID NOs: 235, 236 and 257 as torch oligonucleotides. SEQ ID NOs: 259 and 260 were used as target sequences (30 copies/mL and 300 copies/mL), sample transport media alone was used as negative control, and each condition was run in replicates of five (5). Background signal cutoff was 1,000 RFU. The reactions were set up and run as is generally described above for multiplex, biphasic, real-time TMA reactions.

As shown in the results below, the average fluorescent signal was higher in the FAM channel than in the ROX channel. The condition containing SEQ ID NO: 235 molecular torch showed elevated backgrounds in both of the ROX and FAM channels. Conditions containing SEQ ID NO: 236 or SEQ ID NO: 257 showed lower background signal than did the condition containing SEQ ID NO: 235. The condition containing SEQ ID NO: 236 molecular torch showed the best overall performance in this experiment with strong average fluorescent output and low background.

TABLE 6-1

| | FAM Channel | FAM Channel | ROX Channel | ROX Channel |
|---|---|---|---|---|
| Conditions* | Avg TTime (min) | Avg RFU | Avg TTime (min) | Avg RFU |
| Torch | 30 c/mL = 22.7 | 30 c/mL = 9,806 | 30 c/mL = 0 | 30 c/mL = 219 |
| SEQ ID NO: 235 | 300 c/mL = 20.0 | 300 c/mL = 10,145 | 300 c/mL = 0 | 300 c/mL = 234 |
| | 30 c/mL = 29.0 | 30 c/mL = 1,400 | 30 c/mL = 21.3 | 30 c/mL = 4,155 |
| | 300 c/mL = 28.2 | 300 c/mL = 2,625 | 300 c/mL = 19.6 | 300 c/mL = 4,273 |
| | 0 c/mL = 0 | 0 c/mL = 264 | 0 c/mL = 0 | 0 c/mL = 286 |
| Torch | 30 c/mL = 23.1 | 30 c/mL = 9,824 | 30 c/mL = 0 | 30 c/mL = 60 |
| SEQ ID NO: 236 | 300 c/mL = 20.5 | 300 c/mL = 9,522 | 300 c/mL = 0 | 300 c/mL = 46 |
| | 30 c/mL = 0 | 30 c/mL = 114 | 30 c/mL = 21.6 | 30 c/mL = 3,610 |
| | 300 c/mL = 0 | 300 c/mL = 144 | 300 c/mL = 20.1 | 300 c/mL = 3,681 |
| | 0 c/mL = 0 | 0 c/mL = 301 | 0 c/mL = 0 | 0 c/mL = 97 |
| Torch | 30 c/mL = 22.7 | 30 c/mL = 9,876 | 30 c/mL = 0 | 30 c/mL = 83 |
| SEQ ID NO: 257 | 300 c/mL = 20.5 | 300 c/mL = 9,653 | 300 c/mL = 0 | 300 c/mL = 67 |
| | 30 c/mL = 0 | 30 c/mL = 104 | 30 c/mL = 21.5 | 30 c/mL = 2,873 |
| | 300 c/mL = 0 | 300 c/mL = 218 | 300 c/mL = 20.0 | 300 c/mL = 2,775 |
| | 0 c/mL = 0 | 0 c/mL = 10 | 0 c/mL = 0 | 0 c/mL = 31 |

*Each condition contained the same collection of oligonucleotides as described in this example except for the varied torch shown in this table. TTime and RFU Data cells recite, in descending order 30 copies/mL of SEQ ID NO: 259, 300 copies/mL of SEQ ID NO: 259, 30 copies/mL of SEQ ID NO: 260 and 300 copies/mL of SEQ ID NO: 260.

### Example 7. Singleplex Reactions Having Varied Oligonucleotides for the Amplification and Detection of Influenza A

A series of reagents were prepared to contain various T7 promoter primers, non-T7 primers and torch oligonucleotides for singleplex amplification and detection reactions targeting influenza A. Each singleplex reaction was a biphasic, real-time TMA reaction prepared using a target capture reagent, amplification reagent and promoter reagent, as is described herein. The oligonucleotides used in these reagents were as follows:

TABLE 7-1

| key | T7 Promoter Primer |
|---|---|
| 1 | SEQ ID NO: 30 |
| 2 | SEQ ID NO: 29 |
| 3 | SEQ ID NO: 31 |
| 4 | SEQ ID NO: 28 |
| 5 | SEQ ID NO: 32 |
| 6 | SEQ ID NO: 33 |
| | Non-T7 Primer |
| a | SEQ ID NO: 5 |
| b | SEQ ID NO: 4 |
| c | SEQ ID NO: 3 |
| d | SEQ ID NO: 2 |
| | Torch |
| i | SEQ ID NO: 56 |
| ii | SEQ ID NO: 59 |
| iii | SEQ ID NO: 58 |
| iv | SEQ ID NO: 57 |

Two plates were set up with the following oligonucleotide configuration (using the key from the above table), one plate containing an influenza A H3N2 target nucleic acid represented by the IVT SEQ ID NO: 263, another plate containing an influenza A H1N1 target nucleic acid represented by SEQ ID NO: 262.

TABLE 7-2

| | 1 | 2 | 3 | 4 | 5 | 6 | 7 | 8 | 9 | 10 | 11 | 12 |
|---|---|---|---|---|---|---|---|---|---|---|---|---|
| A | 1ai | 2ai | 3ai | 4ai | 5ai | 6ai | 1aii | 2aii | 3aii | 4aii | 5aii | 6aii |
| B | 1bi | 2bi | 3bi | 4bi | 5bi | 6bi | 1bii | 2bii | 3bii | 4bii | 5bii | 6bii |
| C | 1ci | 2ci | 3ci | 4ci | 5ci | 6ci | 1cii | 2cii | 3cii | 4cii | 5cii | 6cii |
| D | 1di | 2di | 3di | 4di | 5di | 6di | 1dii | 2dii | 3dii | 4dii | 5dii | 6dii |
| E | 1aiii | 2aiii | 3aiii | 4aiii | 5aiii | 6aiii | 1aiv | 2aiv | 3aiv | 4aiv | 5aiv | 6aiv |
| F | 1biii | 2biii | 3biii | 4biii | 5biii | 6biii | 1biv | 2biv | 3biv | 4biv | 5biv | 6biv |
| G | 1ciii | 2ciii | 3ciii | 4ciii | 5ciii | 6ciii | 1civ | 2civ | 3civ | 4civ | 5civ | 6civ |
| H | 1diii | 2diii | 3diii | 4diii | 5diii | 6diii | 1div | 2div | 3div | 4div | 5div | 6div |

Results are presented below and show that torch iii performed well with the H3N2 target nucleic acid, but it showed weak performance with the H1N1 target nucleic acid. Similarly, torches ii and iv performed well with the H3N2 target nucleic acid, but only torch ii performed well with the H1N1 target nucleic acid. The non-T7 primers a-d performed well in all combinations and with both target nucleic acids. The T7 promoter primers 2, 3, 4, and 6 performed well with both target nucleic acids, with T7 promoter primer 4 showing the best performance. T7 promoter primer 1 showed weak performance and T7 promoter primer 5 failed to perform in all combinations showing high TTimes and low RFU.

TABLE 7-3

| | SEQ ID | SEQ ID | SEQ ID | SEQ ID |
|---|---|---|---|---|
| Oligo | NO:263 | NO:263 | NO:262 | NO:262 |
| Combination | RFU | TTime | RFU | TTime |
| 1ai | 39,024 | 10.17 | 17,641 | 13.15 |
| 2ai | 39,029 | 8.66 | 17,430 | 10.54 |
| 3ai | 40,858 | 8.54 | 17.850 | 11.47 |
| 4ai | 39,041 | 7.02 | 16.401 | 8.40 |
| 5ai | 31,925 | 39.57 | 6.311 | 42.08 |
| 6ai | 39,763 | 7.68 | 15,946 | 10.91 |
| 1aii | 41,583 | 10.26 | 24,398 | 13.81 |
| 2aii | 42,105 | 8.45 | 24.095 | 11.23 |
| 3aii | 42,462 | 9.00 | 22,267 | 11.70 |
| 4aii | 42,380 | 6.98 | 22,115 | 8.76 |
| 5aii | 35,042 | 35.52 | 13,536 | 41.80 |
| 6ai | 41,805 | 8.16 | 19.013 | 11.40 |
| 1bi | 40,187 | 9.72 | 16,874 | 13.11 |
| 2bi | 40,475 | 8.51 | 16.045 | 10.17 |
| 3bi | 39,749 | 9.01 | 16,644 | 10.54 |
| 4bi | 39,723 | 6.98 | 16.665 | 8.33 |
| 5bi | 39,544 | 37.09 | 12,372 | 40.23 |
| 6bi | 40,368 | 8.16 | 16,958 | 10.29 |
| 1bii | 42,769 | 10.50 | 20,517 | 13.60 |
| 2bii | 42,979 | 8.18 | 24,653 | 10.47 |
| 3bii | 41,670 | 9.12 | 23,087 | 10,99 |
| 4bii | 42,821 | 7.01 | 22.037 | 8.67 |
| 5bii | 40,447 | 35.16 | 19.119 | 39.08 |
| 6bii | 42,015 | 8.00 | 19.064 | 10.51 |
| 1ci | 40,191 | 11,06 | 18.041 | 13.62 |
| 2ci | 40,635 | 8.13 | 17,170 | 10.45 |
| 3ci | 41,804 | 9.75 | 17.614 | 11.36 |
| 4ci | 41,568 | 7.44 | 16,885 | 8.96 |
| 5ci | 41,432 | 36.25 | 11,785 | 40.13 |
| 6ci | 39,123 | 7.93 | 21,554 | 9.30 |
| 1cii | 41,835 | 10.69 | 20,798 | 14.09 |
| 2cii | 42,592 | 8.44 | 20,710 | 10.44 |
| 3cii | 42,562 | 9.59 | 22.685 | 11.40 |
| 4cii | 42,883 | 7.19 | 15,237 | 8.91 |
| 5cii | 38,549 | 37.80 | 14,636 | 39.79 |
| 6cii | 42,573 | 7.68 | 20,487 | 9.63 |
| 1di | 39,402 | 10.58 | 15,323 | 12.82 |
| 2di | 39,864 | 8.70 | 15.609 | 10.27 |
| 3di | 40,430 | 9.13 | 16.436 | 10.50 |
| 4di | 37,019 | 7.48 | 16.234 | 7.93 |
| 5di | 38,089 | 36.91 | 13,304 | 40.46 |
| 6di | 40,695 | 8.11 | 16,701 | 9.91 |
| 1dii | 40,452 | 11.12 | 19.087 | 13.56 |
| 2dii | 42,565 | 8.93 | 20,784 | 10.36 |
| 3dii | 40,759 | 9.59 | 22.592 | 11.44 |
| 4dii | 42,874 | 7.46 | 22,050 | 8.66 |
| 5dii | 40,387 | 37.01 | 18,134 | 42.58 |
| 6dii | 42,198 | 8.46 | 20,172 | 10.35 |
| laiii | 43,652 | 11.78 | 12.285 | 17.53 |
| 2aiii | 44,772 | 9.76 | 13,975 | 14.24 |
| 3aiii | 46,888 | 10.19 | 14.102 | 14.99 |
| 4aiii | 48,785 | 7.65 | 15,208 | 11.84 |
| 5aiii | 33,639 | 40.31 | 1.371 | 45.30 |
| 6aiii | 45,992 | 9.07 | 13,030 | 15.08 |
| laiv | 34,402 | 10.22 | 22.914 | 13.34 |
| 2aiv | 34,580 | 9.15 | 22.554 | 10.96 |
| 3aiv | 35,891 | 9.23 | 21,387 | 11.52 |
| 4aiv | 35,396 | 7.22 | 19,174 | 9.26 |
| 5aiv | 26,562 | 37.60 | 7,758 | 42.54 |
| 6aiv | 35,987 | 8.16 | 21,203 | 10,49 |
| 1biii | 42,224 | 11.61 | 14.252 | 17.34 |
| 2biii | 45,004 | 9.52 | 14,147 | 13.19 |
| 3biii | 46,044 | 10.02 | 13,898 | 14.41 |
| 4biii | 46,690 | 7.95 | 14.996 | 11.14 |
| 5biii | 42,449 | 38.86 | 3.014 | 42.68 |
| 6biii | 45,958 | 8.94 | 14,911 | 13.66 |
| 1biv | 35,397 | 10.09 | 18,286 | 12.86 |
| 2biv | 34,724 | 8.16 | 17.334 | 10.30 |
| 3biv | 35,383 | 9.02 | 19.566 | 10.88 |
| 4biv | 35,338 | 7.03 | 18.863 | 8.69 |
| 5biv | 31,000 | 35.51 | 12.857 | 42.09 |
| 6biv | 35,562 | 8.07 | 19,466 | 10.18 |
| 1ciii | 37,376 | 12,28 | 11,699 | 19.36 |
| 2ciii | 43,184 | 9.64 | 12,747 | 14.63 |
| 3ciii | 47,397 | 10.73 | 13,062 | 16.68 |
| 4ciii | 46,539 | 8.58 | 14.717 | 12.73 |
| 5ciii | 42,293 | 40.40 | 2.805 | 42.55 |
| 6ciii | 46,776 | 9.07 | 16,623 | 13.85 |
| 1civ | 35,933 | 10.76 | 20,043 | 13.76 |
| 2civ | 36,548 | 8.59 | 21,579 | 10.34 |
| 3civ | 37,503 | 9.55 | 20.085 | 11.67 |
| 4civ | 36,583 | 7.19 | 21.236 | 8.89 |
| 5civ | 30,767 | 37.94 | 11,057 | 41.10 |
| 6civ | 37,883 | 8.12 | 19,506 | 9.61 |
| 1diii | 42,252 | 11.69 | 14.022 | 17.35 |
| 2diii | 41,251 | 9.67 | 13,969 | 13.44 |
| 3diii | 42,890 | 10.56 | 14,610 | 14.75 |
| 4diii | 43,958 | 8.20 | 15.230 | 11.34 |
| 5diii | 41,927 | 38.50 | 4.524 | 45.07 |
| 6diii | 44,164 | 9.18 | 15.132 | 13.98 |
| 1div | 35,907 | 10.26 | 18.631 | 12.92 |
| 2div | 36,109 | 8.70 | 17,979 | 10.20 |
| 3div | 36,528 | 9.16 | 18,162 | 10.85 |
| 4div | 35.968 | 7.45 | 17,744 | 8.41 |
| 5div | 32,029 | 37.45 | 13,240 | 42.68 |
| 6div | 36,023 | 8.11 | 17.463 | 9.44 |

### Example 8. Singleplex Reactions Having Varied Oligonucleotides for the Amplification and Detection of Influenza B

A series of reagents were prepared to contain various T7 promoter primers, non-T7 primers and torch oligonucleotides for singleplex amplification and detection reactions targeting influenza B. Each singleplex reaction was a biphasic, real-time TMA reaction prepared using a target capture reagent, amplification reagent and promoter reagent, as is described herein. The oligonucleotides used in these reagents were as follows:

TABLE 8-1

| key | T7 Promoter Primer |
|---|---|
| 1 | SEQ ID NO: 99 |
| 2 | SEQ ID NO: 98 |
| 3 | SEQ ID NO: 97 |
| 4 | SEQ ID NO: 96 |
| | Non-T7 Primer |
| a | SEQ ID NO: 74 |
| b | SEQ ID NO: 72 |
| c | SEQ ID NO: 75 |
| | Torch |
| i | SEQ ID NO: 114 |
| ii | SEQ ID NO: 116 |
| iii | SEQ ID NO: 115 |

A plate was set up with the following oligonucleotide configuration (using the key from the above table), one plate containing an influenza B target nucleic acid represented by the IVT SEQ ID NO: 264.

TABLE 8-2

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | |
|---|---|---|---|---|---|---|---|---|
| A | 1ai | 2ai | 3ai | 4ai | 1aii | 2aii | 3aii | 4aii |
| B | 1bi | 2bi | 3bi | 4bi | 1bii | 2bii | 3bii | 4bii |
| C | 1ci | 2ci | 3ci | 4ci | 1cii | 2cii | 3cii | 4cii |
| D | | | | | | | | |
| E | 1aiii | 2aiii | 3aiii | 4aiii | | | | |
| F | 1biii | 2biii | 3biii | 4biii | | | | |
| G | 1ciii | 2ciii | 3ciii | 4ciii | | | | |
| H | | | | | | | | |

Results are presented below and show that all combinations had fast TTimes, some under 10 minutes, and combination 2aii under 5 minutes. Torches 2 and 3 showed the strongest amplification curves (not shown here) and combinations using these torches all had RFU values above 20,000 (except combination 4ciii).

TABLE 8-3

| | SEQ ID | SEQ ID |
|---|---|---|
| Oligo | NO:264 | NO:264 |
| Combination | TTime | RFU |
| 1ai | 5.90 | 23,975 |
| 2ai | 5.49 | 14,061 |
| 3ai | 7.23 | 14,942 |
| 4ai | 7.95 | 11,880 |
| 1aii | 7.83 | 36,387 |
| 2aii | 4.25 | 34,325 |
| 3aii | 9.65 | 38,713 |
| 4aii | 8.90 | 41,465 |
| 1aiii | 8.04 | 35,843 |
| 2aiii | 6.35 | 26,395 |
| 3aiii | 9.91 | 28,263 |
| 4aiii | 8.97 | 27,640 |
| 1bi | 6.46 | 19,878 |
| 2bi | 5.58 | 17,902 |
| 3bi | 10.01 | 14,250 |
| 4bi | 10.05 | 9,585 |
| 1bii | 8.37 | 27,015 |
| 2bii | 6.68 | 20,593 |
| 3bii | 9.65 | 36,481 |
| 4bii | 9.88 | 39,104 |
| 1biii | 8.55 | 23,786 |
| 2biii | 6.97 | 19,218 |
| 3biii | 10.14 | 21,771 |
| 4biii | 10.03 | 21,537 |
| 1ci | 7.90 | 19,917 |
| 2ci | 5.32 | 20,077 |
| 3ci | 9.76 | 19,041 |
| 4ci | 8.35 | 18,476 |
| 1cii | 8.27 | 29,696 |
| 2cii | 5.91 | 26,814 |
| 3cii | 9.99 | 30,212 |
| 4cii | 9.42 | 36,071 |
| 1ciii | 8.56 | 25,493 |
| 2ciii | 6.40 | 24,054 |
| 3ciii | 10.76 | 28,267 |
| 4ciii | 9.42 | 15,104 |

### Example 9. Multiplex Assay to Determine the Performance of Primers and Probes for the Detection of SARS-CoV-2 in the Presence of Target Capture Oligomers, Primers and Probes for the Detection of Each of Influenza A and Influenza B

A multiplex reaction was prepared to test the performance of primers and probes for amplifying and detecting two regions of a SARS-CoV-2 target nucleic acid in the presence of primers and probes for detecting influenza target nucleic acids. The oligonucleotides used in this experiment were as follows: in the target capture reagent was SEQ ID NOs: 48, 108, 223, and 224 (target capture oligonucleotides) and SEQ ID NOs: 28, 98, 169, and 172 (T7 promoter primers); in the amplification reagent was SEQ ID NOs: 1, 72, 126, and 127 (non-T7 primers); and in the promoter reagent was SEQ ID NOs: 28, 98, 167, and 172 (T7 promoter primers) and SEQ ID NOs: 54, 55, 116, 234, and 235 (torch oligomers). SEQ ID NOs: 259 and 260 were used as target nucleic acids (5 copies/mL, 10 copies/mL, 20 copies/mL, 30 copies/mL, and 100 copies/mL). Negative reactions were sample transport media alone. Each reaction condition was run in replicates of twenty (20).

TABLE 9-1

| | SEQ ID NO: 259 | SEQ ID NO: 260 |
|---|---|---|
| 0 copies/mL | 0% (0/20) | 0% (0/20) |
| 5 copies/mL | 70% (14/20) | 75% (15/20) |
| 10 copies/mL | 95% (19/20) | 80% (16/20) |
| 20 copies/mL | 95% (19/20) | 100% (20/20) |
| 30 copies/mL | 100% (20/20) | 95% (19/20) |
| 100 copies/mL | 100% (20/20) | 100% (20/20) |

In this example, the limit of detection for the multiplex reaction is 13.9 copies/mL of SEQ ID NO: 259 and 17.2 copies/mL of SEQ ID NO: 260, as determined by Probit analysis with a 95% probability.

### Example 10. Multiplex Assay to Determine the Performance of Primers and Probes for the Detection of SARS-CoV-2 and Influenza A

A multiplex reaction was prepared to test the performance of primers and probes for amplifying and detecting two regions of a SARS-CoV-2 target nucleic acid in the presence of primers and probes for detecting influenza A target nucleic acids. The oligonucleotides used in this experiment were as follows: in the target capture reagent was SEQ ID NOs: 50, 223, and 227 (target capture oligonucleotides) and SEQ ID NOs: 28, 169, and 183 (T7 promoter primers); in the amplification reagent was SEQ ID NOs: 4, 126, and 134 (non-T7 primers); and in the promoter reagent was SEQ ID NOs: 28, 167, and 194 (T7 promoter primers) and SEQ ID NOs: 57, 234, and 243 (torch oligomers—all FAM labeled). SEQ ID NOs: 259, 261, 262, and 263 were used as target nucleic acids (30 copies/μL and 100 copies/μL). Negative reactions were sample transport media alone. Each reaction condition was run in replicates of five (5). Results for this assay were as follows (reporting average TTimes): SEQ ID NO: 259 at 30 c/μL=17.7 min and at 100 c/μL=16.7 min; SEQ ID NO: 261 at 30 c/μL=13.22 min and at 100 c/μL=12.1 min; SEQ ID NO: 262 at 30 c/μL=20.2 min and at 100 c/μL=18.4 min; and SEQ ID NO: 263 at 30 c/μL=19.5 min and at 100 c/μL=17.7 min. These results show that the multiplex system has a fast TTime. Amplification reaction curves (not shown) showed robust amplification for each of the SARS-CoV-2 target nucleic acids. Amplification reaction curves for both influenza A target nucleic acids showed inconsistent amplification curves for each of the replicates (“fanning”), which indicates a less robust amplification reaction.

### Example 11. Multiplex Assay to Determine the Performance of the Assay in the Presence of Challenge Organisms

The purpose of this experiment was to test the performance of a multiplex assay for the amplification and detection of SARS-CoV-2, influenza A, and influenza B in the presence of 15 different pools of challenge organisms. The multiplex assay was set-up and performed as a biphasic, real-time TMA reaction, as is described above. Oligonucleotides were as follows: SEQ ID NOs: 48, 108, 223, and 224 as target capture oligonucleotides; SEQ ID NOs: 28, 98, 167, 169, and 172 as T7 promoter primers; SEQ ID NOs: 1, 72, 126, and 127 as non-T7 primers; and SEQ ID NOs: 54, 55, 116, 234, and 235 as torch oligomers.

A target capture oligonucleotide, T7 promoter primer, non-T7 primer, and a torch oligonucleotide configured to target an internal control nucleic acid were included (sequences not shown). The oligonucleotides were combined into a target capture reagent, an amplification reagent, and a promoter reagent, as described above. Torch oligomers targeting the SARS-CoV-2 target nucleic acids were labeled with FAM/Dabcyl. Torch oligomers targeting the influenza A target nucleic acids were labeled with ROX/Acridine. Torch oligomers targeting the influenza B target nucleic acids were labeled with HEX/Dabcyl. Torch oligomers targeting the internal control were labeled with Cy5/Blackberry Quencher. Fifty-nine (59) challenge organisms were grouped into 15 pools, as is shown below. Negative control wells were sample transport media only.

TABLE 11-1

| | | Target | |
|---|---|---|---|
| Pool Number | Microorganisms | Concentration | Units |
| Neg Control | Sample Transport Media | n/a | n/a |
| SARS/Flu IVTs | SEQ ID NOs: 259, 260, 262, 263, | | |
| | and264 | | |
| SARS IVT | SEQ ID NO: 259 | 100 | copies/mL |
| SARS IVT | SEQ ID NO: 260 | 100 | copies/mL |
| 1 | Adenovirus 1* | 1.00E+06 | TCID50/mL |
| | Adenovirus 7a* | 1.00E+04 | TCID50/mL |
| | CMV Strain AD 169 | 5.00E+03 | TCID50/mL |
| 2 | Bordetella bronchiseptica | 1.00E+06 | CFU/mL |
| | Bordetella pertussis | 1.00E+06 | CFU/mL |
| | Candida albicans | 1.00E+06 | CFU/mL |
| | Chlamydia pneumonia | 1.00E+05 | CFU/mL |
| | Chlamydia trachomatis** | 1.00E+06 | CFU/mL |
| 3 | Coxsackie B4 | 1.00E+03 | TCID50/mL |
| | Coxsackie B5/10/2006 | 1.00E+05 | TCID50/mL |
| 4 | Corynebacterium diphtheria | 1.00E+06 | CFU/mL |
| | E. coli | 2.00E+06 | CFU/mL |
| | Haemophilus influenzae | 1.00E+06 | CFU/mL |
| 5 | Echovirus 11* | 1.00E+06 | TCID50/mL |
| | Echovirus 2* | 1.00E+06 | TCID50/mL |
| | Echovirus 3 | 1.00E+04 | TCID50/mL |
| | Echovirus 6 | 5.00E+04 | TCID50/mL |
| 6 | EBV** | 1.00E+06 | TCID50/mL |
| | Enterovirus (e.g. EV68) | 1.00E+06 | TCID50/mL |
| | Rhinovirus | 1.00E+04 | TCID50/mL |
| | Varicella Zoster Virus | 5.00E+03 | TCID50/mL |
| 7 | Human coronavirus 229E | 1.00E+07 | TCID50/mL |
| | Human coronavirus HKU1 | 1.00E+06 | c/mL |
| | Human coronavirus NL63 | 1.00E+03 | TCID50/mL |
| | Human coronavirus OC43 | 1.00E+04 | TCID50/mL |
| | MERS | 1.00E+03 | TCID50/mL |
| | SARS | 1.00E+06 | c/mL |
| 8 | HPIV-1\hat{ }\hat{ } | 1.00E+06 | TCID50/mL |
| | HPIV-2 | 1.00E+06 | TCID50/mL |
| | HPIV-3* | 1.00E+05 | TCID50/mL |
| | HPIV-4a | 2.00E+03 | TCID50/mL |
| 9 | hMPV-9 Subtype A1 | 1.00E+06 | TCID50/mL |
| | HSV-1 Macinytre Strain | 5.00E+05 | TCID50/mL |
| | RSV A\hat{ } | 1.00E+05 | TCID50/mL |
| | RSV B\hat{ } ch93(18) | 1.00E+06 | TCID50/mL |
| 10 | Klebsiella pneumonia | 1.00E+06 | CFU/mL |
| | Lactobacillus plantarum | 1.00E+06 | CFU/mL |
| | Legionella pneumophila | 1.00E+06 | CFU/mL |
| | Tatlockia micdadei (Legionella | 1.00E+06 | CFU/mL |
| | micdadei) | | |
| 11 | Measles/7/2000\hat{ }\hat{ } | 1.00E+04 | TCID50/mL |
| | Mumps virus\hat{ }\hat{ } | 1.00E+06 | TCID50/mL |
| 12 | Moraxella catarrhalis | 1.00E+06 | CFU/mL |
| | Mycobacterium intracellulare | 1.00E+08 | rRNA c/mL |
| | Mycobacterium tuberculosis | 1.00E+09 | CFU/mL |
| | Mycoplasma pneumoniae | 1.00E+06 | CFU/mL |
| 13 | Neisseria gonorrhea | 1.00E+06 | CFU/mL |
| | Neisseria meningitides | 1.00E+06 | CFU/mL |
| | Neisseria mucosa | 1.00E+06 | CFU/mL |
| 14 | Pneumocystis jirovecii (PJP) | 1.00E+06 | CFU/mL |
| | Proteus mirabilis | 1.00E+06 | CFU/mL |
| | Proteus vulgaris | 1.00E+06 | CFU/mL |
| | Pseudomonas aeruginosa | 1.00E+06 | CFU/mL |
| 15 | Staphlycoccus aureus | 1.00E+06 | CFU/mL |
| | Staphlycoccus epidermidis | 1.00E+06 | CFU/mL |
| | Streptococcus pneumonia | 1.00E+05 | CFU/mL |
| | Streptococcus pyogenes | 1.00E+06 | CFU/mL |
| | Streptococcus salivarius | 1.00E+06 | CFU/mL |

The negative control, triple positive control, and the two SARS control conditions were each tested in replicates of five (5). Each pool of challenge organisms was tested in a single replicate. Results are presented below as RFU and TTime for each condition (averaged for conditions tested in multiple replicates).

TABLE 11-2

| | FAM Channel | | HEX Channel | | ROX Channel | | Cy5.5 Channel | |
|---|---|---|---|---|---|---|---|---|
| | RFU | TTime | RFU | TTime | RFU | TTime | RFU | TTime |
| Panel | (Avg) | (Avg) | (Avg) | (Avg) | (Avg) | (Avg) | (Avg) | (Avg) |
| Neg Control | 78.68 | | −267.12 | | −237.08 | | 3364.88 | 16.51 |
| SARS/Flu IVTs | 13850.76 | 18.52 | 5325.32 | 23.45 | 7099.96 | 21.95 | 3197.64 | 16.59 |
| SARS IVT | 6343.92 | 18.23 | −231.76 | | −248.52 | | 3232.80 | 16.55 |
| SARS IVT | 6930.88 | 20.72 | −214.00 | | −239.88 | | 3193.84 | 16.53 |
| POOL1 | 89.60 | | −274.80 | | −209.00 | | 3564.20 | 16.70 |
| POOL2 | 69.00 | | −316.20 | | −259.20 | | 3487.40 | 16.26 |
| POOL3 | 30.60 | | −247.60 | | −264.20 | | 3169.60 | 16.59 |
| POOL4 | 56.80 | | −243.80 | | −206.40 | | 3035.40 | 16.62 |
| POOL5 | 50.80 | | −301.40 | | −258.00 | | 3060.00 | 16.72 |
| POOL6 | 83.00 | | −247.80 | | −195.00 | | 3980.80 | 16.44 |
| POOL7 | 82.60 | | −295.40 | | −239.40 | | 3688.80 | 16.62 |
| POOL8 | −30.60 | | −311.80 | | −333.20 | | 3493.40 | 16.53 |
| POOL9 | 55.60 | | −309.40 | | −297.40 | | 3663.60 | 16.73 |
| POOL10 | 41.80 | | −312.00 | | −262.80 | | 3154.80 | 16.44 |
| POOL11 | 107.60 | | −300.00 | | 288.40 | | 3921.40 | 16.41 |
| POOL12 | 72.00 | | −265.40 | | −210.60 | | 3545.80 | 16.27 |
| POOL13 | 79.60 | | −278.80 | | −237.20 | | 3668.40 | 16.39 |
| POOL14 | 37.00 | | −299.40 | | −282.60 | | 3614.20 | 16.75 |
| POOL15 | 59.40 | | −339.00 | | −287.20 | | 3776.80 | 16.39 |

There were no false positive results in any of the fifteen (15) pools of challenge organisms. The control reactions (negative control, triple positive control, and single positive SARS-CoV-2 controls) performed as expected showing robust positive signals, fast TTimes, and no false positives.

### Example 12. Probe Oligonucleotides for Detecting Sars-CoV-2, Region 1

Additional SARS-CoV-2 probe oligomers, (R1 Torch 9 (SEQ ID NO: 270), R1 Torch 10, (SEQ ID NO: 269) and R1 Torch 11 (SEQ ID NO: 268), that bind SARS-CoV-2 region 1 amplicon were generated and tested. Each of R1 Torches 9, 10, and 11 have a 7 bp stem loop structure. R1 Torches 9, 10, and 11 each also contain a 3′ terminal GC or CG dinucleotide. Samples were prepared and tested as in Example 9 using several oligonucleotide combinations comprising SEQ ID NOs: 28, 25, 54, 55, 98, 167, 172, 116, and 235 combined with one of SEQ ID NOs: 268, 269, or 270. Amplification were performed as described above using 34 mM MgCl₂ and 10% nucleotides. Samples were run on a Panther system. In addition, the samples contained oligonucleotides for amplifying and detecting an internal control.

TABLE 12-1
Multiplex assay containing SAR-COV-2 probe R1 Torch 9.

| | | | Oligo |
|---|---|---|---|
| Reagent | Target | Description | type |
| RI Torch 9 | Region 1 | SEQ ID NO:167 | T7 |
| | | SEQ ID NO:270 | Torch |
| | Region 2 | SEQ ID N:172 | T7 |
| | | SEQ ID NO:235 | Torch |
| | Flu A | SEQ ID NO:28 | T7 |
| | | SEQ ID NO:55 | Torch |
| | | SEQ ID NO:54 | Torch |
| | Flu B | SEQ ID NO:98 | T7 |
| | | SEQ ID NO:116 | Torch |

TABLE 12-2
Multiplex assay containing SAR-COV-2 probe R1 Torch 10.

| | | | Oligo |
|---|---|---|---|
| Reagent | Target | Description | type |
| R1 Torch 10 | Region 1 | SEQ ID NO:167 | T7 |
| | | SEQ ID NO:269 | Torch |
| | Region 2 | SEQ ID N:172 | T7 |
| | | SEQ ID NO:235 | Torch |
| | Flu A | SEQ ID NO:28 | T7 |
| | | SEQ ID NO:55 | Torch |
| | | SEQ ID NO:54 | Torch |
| | Flu B | SEQ ID NO:98 | T7 |
| | | SEQ ID NO:116 | Torch |

TABLE 12-3
Multiplex assay containing SAR-COV-2 probe R1 Torch 11.

| | | | Oligo |
|---|---|---|---|
| Reagent | Target | Description | type |
| R1 Torch 11 | Region 1 | SEQ ID NO:167 | T7 |
| | | SEQ ID NO:268 | Torch |
| | Region 2 | SEQ ID N:172 | T7 |
| | | SEQ ID NO:235 | Torch |
| | Flu A | SEQ ID NO:128 | T7 |
| | | SEQ ID NO:55 | Torch |
| | | SEQ ID NO:54 | Torch |
| | Flu B | SEQ ID NO:98 | T7 |
| | | SEQ ID NO:116 | Torch |

TABLE 12-4
Detection using FAM flourophore

| | | | | | RFU Range | | |
|---|---|---|---|---|---|---|---|
| Panel | Target | Conc. | Units | Condition | N | Mean | Std Dev |
| C33A_1e5 | C33A | 1.00E+05 | cells/mL | TORCH 9 | 10 | −71.26 | 10.92 |
| | | | | TORCH 10 | 10 | −101.00 | 17.60 |
| | | | | TORCH 11 | 10 | −113.62 | 10.51 |
| PostCtrl | Wuhan CoV-1 Transcript | 1.50E+03 | cp/mL | TORCH 9 | 5 | 4054.92 | 387.16 |
| | Flu A H1N1 Transcript | 4.40E+03 | cp/mL | TORCH 10 | 5 | 3912.73 | 224.14 |
| | Flu B M1 Transcript | 5.70E+03 | cp/mL | TORCH 11 | 5 | 5936.68 | 544.29 |
| QC2 | Wuhan CoV-2 Transcript | | cp/mL | TORCH 9 | 5 | 7036.08 | 443.45 |
| | | 1.50E+04 | | TORCH 10 | 5 | 5615.80 | 308.03 |
| | | | | TORCH 11 | 5 | 6772.04 | 386.28 |
| R1_3xLoD | Wuhan CoV-1 Transcript | 42 | cp/mL | TORCH 9 | 5 | 3578.68 | 492.20 |
| | | | | TORCH 10 | 5 | 4017.28 | 329.21 |
| | | | | TORCH 11 | 5 | 5458.20 | 434.44 |
| STM | none | | | TORCH 9 | 5 | −68.36 | 6.11 |
| | | | | TORCH 10 | 5 | −111.04 | 24.65 |
| | | | | TORCH 11 | 5 | −120.40 | 6.76 |

All three candidates had low background with the C33A panels, a human HPV cell line that does not contain SARS-CoV-2. All three probe oligo nucleotides specifically detected SARS-CoV-2 in samples containing the virus. Of the three Region 1 SARS-CoV-2 probe oligonucleotides tested in this example. R1 Torch 11 had the highest specific signal.

### Example 13. Probe Oligonucleotides for Detecting Sars-CoV-2, Region 1

SARS-CoV-2 probe oligomers, (R1 Torch 3 (SEQ ID NO: 234), R1 Torch 4 (SEQ ID NO: 266), R1 Torch 7, (SEQ ID NO: 267) and R1 Torch 11 (SEQ ID NO: 268), which bind SARS-CoV-2 region 1 amplicon, were tested in multiplex biphasic TMA reactions. Samples were prepared and tested as in Example 9 using AMP mixture of example 9 and several oligonucleotide combinations comprising SEQ ID NOs: 28, 25, 54, 55, 98, 167, 172, 116, and 235 combined with one of SEQ ID NOs: 266, 267, or 268. Amplification reactions were performed as above using 34 mM MgCl₂+10% nucleotides. Samples were run on a Panther system. In addition, the samples contained oligonucleotides for amplifying and detecting an internal control. The probe oligomers were tested in the presence of higher levels of human transcriptome to analyze background and false positivity rate. 318 reps were run form R1 Torch 4 and R1 Torch 7. 212 reps were run form R1 Torch 11.

TABLE 13-1

| | | | Oligo |
|---|---|---|---|
| Reagent | Target | Description | type |
| RI Torch 4 | Region 1 | SEQ ID NO:167 | T7 |
| | | SEQ ID NO:266 | Torch |
| | Region 2 | SEQ ID NO:172 | T7 |
| | | SEQ ID NO:235 | Torch |
| | Flu A | SEQ ID NO:28 | T7 |
| | | SEQ ID NO:55 | Torch |
| | | SEQ ID NO:54 | Torch |
| | Flu B | SEQ ID NO:98 | T7 |
| | | SEQ ID NO:116 | Torch |

TABLE 13-2

| | | | Oligo |
|---|---|---|---|
| Reagent | Target | Description | type |
| R1 Torch 7 | Region 1 | SEQ ID NO:167 | T7 |
| | | SEQ ID NO:267 | Torch |
| | Region 2 | SEQ ID NO:172 | T7 |
| | | SEQ ID NO:235 | Torch |
| | Flu A | SEQ ID NO:28 | T7 |
| | | SEQ ID NO:55 | Torch |
| | | SEQ ID NO:54 | Torch |
| | Flu B | SEQ ID NO:98 | T7 |
| | | SEQ ID NO:116 | Torch |

TABLE 13-3

| | | | Oligo |
|---|---|---|---|
| Reagent | Target | Description | type |
| R1 Torch 11 | Region 1 | SEQ ID NO:167 | T7 |
| | | SEQ ID NO:268 | Torch |
| | Region 2 | SEQ ID NO:172 | T7 |
| | | SEQ ID NO:235 | Torch |
| | Flu A | SEQ ID NO:28 | T7 |
| | | SEQ ID NO:55 | Torch |
| | | SEQ ID NO:54 | Torch |
| | Flu B | SEQ ID NO:98 | T7 |
| | | SEQ ID NO:116 | Torch |

R1 Torch 3, R1 Torch 4, R1 Torch 7, and R1 Torch 11 were each effective in detecting SARS. R1 Torch 3 exhibited a higher false positive rate than R1 Torch 4, R1 Torch 7, and R1 Torch 11 (Table 13-4). R1 Torches 4, 7, and 11 also had lower background and less fanning than Torch 3. R1 Torches 4 and 11 exhibited less background (e.g., fanning) R1 Torch 7. R1 Torch 4 exhibited the least false positive. However, all four torches were effective in detecting SARS-CoV-2.

TABLE 13-4
FAM Detection of SARS from MTS-Insource samples using
various Region 1 SARS-COV-2 probe oligonucleotides.

| | Total | No. of | False Positive | RFU range | |
|---|---|---|---|---|---|
| Condition | Samples Run | Positives | Rate | Mean | Std Dev |
| R1 Torch 3 | 1162 | 62 | 5.34% | 280.39 | 1097.97 |
| R1 Torch 4 | 318 | 3 | 0.94% | −49.75 | 590.11 |
| R1 Torch 7 | 318 | 7 | 2.2% | 55.21 | 944.25 |
| R1 Torch 11 | 212 | 3 | 1.42% | 2.06 | 1181.65 |

1. A composition or kit for determining the presence or absence of SARS-CoV-2, influenza A, and/or influenza B in a sample, said composition or kit comprising one or more of the following primer sets:
(a) a first primer set comprising first and second SARS-CoV-2 region 1 primers capable of amplifying a target region of a SARS-CoV-2 target nucleic acid wherein,
(i) the SARS-CoV-2 region 1 first primer comprises a nucleotide sequence having no more than 0, 1, 2, 3, 4, or 5 mismatches from the nucleotide sequence of SEQ ID NO: 126 or an RNA equivalent or a DNA/RNA chimeric thereof, and
(ii) the SARS-CoV-2 region 1 second primer comprises a target hybridizing region 18-35 nucleobases in length, wherein the target hybridizing region is contained within SEQ ID NO: 140 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 141 or an RNA equivalent or a DNA/RNA chimeric thereof;
(b) a second primer set comprising first and second SARS-CoV-2 region 2 primers capable of amplifying a target region of a SARS-CoV-2 target nucleic acid wherein,
(i) the SARS-CoV-2 region 2 first primer comprises a target hybridizing region 16-30 nucleobases in length, wherein the target hybridizing region is contained within SEQ ID NO: 143 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 144 or an RNA equivalent or a DNA/RNA chimeric thereof, and
(ii) the SARS-CoV-2 region 2 second primer comprises a target hybridizing region 11-35 nucleobases in length, wherein the target hybridizing region is contained within SEQ ID NO: 147, 148, or 149 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 150, 151, or 152 or an RNA equivalent or a DNA/RNA chimeric thereof;
(c) a third primer set comprising first and second SARS-CoV-2 region 3 primers capable of amplifying a target region of a SARS-CoV-2 target nucleic acid wherein,
(i) the SARS-CoV-2 region 3 first primer comprises target hybridizing region 18-30 nucleobases in length, wherein the target hybridizing region is contained within SEQ ID NO: 155 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 156 or 157 or an RNA equivalent or a DNA/RNA chimeric thereof, and
(ii) the SARS-CoV-2 region 3 second primer comprises a target hybridizing region 13-40 nucleobases in length, wherein the target hybridizing region is contained within SEQ ID NO: 160, 161, or 162 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 163, 164, 165, or 166 or an RNA equivalent or a DNA/RNA chimeric thereof;
(d) a fourth primer set comprising first and second Influenza A region 1 primers capable of amplifying a target region of an Influenza A target nucleic acid,
(i) the influenza A region 1 first primer comprises a target hybridizing region 15-30 nucleobases in length, wherein the target hybridizing region is contained within SEQ ID NO:15 or an RNA equivalent or a DNA/RNA chimeric thereof and comprises a nucleotide sequence differing by no more than 0, 1, 2, 3, 4, or 5 nucleotides from the nucleotide sequence of SEQ ID NO:16 or SEQ ID NO:17 an RNA equivalent or a DNA/RNA chimeric thereof, and
(ii) the influenza A region 1 second primer comprises a target hybridizing region 15-30 nucleobases in length, wherein the target hybridizing region is contained within SEQ ID NO:18 an RNA equivalent or a DNA/RNA chimeric thereof and comprises a nucleotide sequence differing by no more than 0, 1, 2, 3, 4, or 5 nucleotides from the nucleotide sequence of SEQ ID NO:19 or SEQ ID NO:20 or an RNA equivalent or a DNA/RNA chimeric thereof;
(e) a fifth primer set comprising first and second Influenza A region 2 primers capable of amplifying a target region of an Influenza A target nucleic acid,
(i) the influenza A region 2 first primer comprises a target hybridizing region 15-30 nucleobases in length, wherein the target hybridizing region is contained within SEQ ID NO: 25 an RNA equivalent or a DNA/RNA chimeric thereof and comprises a nucleotide sequence differing by no more than 0, 1, 2, 3, 4, or 5 nucleotides from the nucleotide sequence of SEQ ID NO: 26 or SEQ ID NO: 27 or an RNA equivalent or a DNA/RNA chimeric thereof, and
(ii) the influenza A region 2 second primer comprises a target hybridizing region 15-30 nucleobases in length, wherein the target hybridizing region is contained within SEQ ID NO: 21 or an RNA equivalent or a DNA/RNA chimeric thereof and comprises a nucleotide sequence differing by no more than 0, 1, 2, 3, 4, or 5 nucleotides from the nucleotide sequence of SEQ ID NO:22 or an RNA equivalent or a DNA/RNA chimeric thereof;
(f) a sixth primer set comprising first and second Influenza B region 1 primers capable of amplifying a target region of an Influenza B target nucleic acid,
(i) the influenza B region 1 first primer comprises a target hybridizing region 20-30 nucleobases in length, wherein the target hybridizing region is contained within SEQ ID NO: 90 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 91 or an RNA equivalent or a DNA/RNA chimeric thereof, and
(ii) the influenza B region 1 second primer comprises a target hybridizing region 17-30 nucleobases in length, wherein the target hybridizing region is contained within SEQ ID NO: 85 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 86 or SEQ ID NO: 87 or an RNA equivalent or a DNA/RNA chimeric thereof; and
(g) a seventh primer set comprising first and second Influenza B region 2 primers capable of amplifying a target region of an Influenza B target nucleic acid,
(i) the influenza B region 2 first primer comprises a target hybridizing region 13-30 nucleobases in length, wherein the target hybridizing region is contained within SEQ ID NO: 83 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 84 or an RNA equivalent or a DNA/RNA chimeric thereof, and
(ii) the influenza B region 2 second primer comprises a target hybridizing region 17-30 nucleobases in length, wherein the target hybridizing region is contained within SEQ ID NO: 79 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 80 or an RNA equivalent or a DNA/RNA chimeric thereof.

2. The composition or kit of claim 1, wherein the composition or kit comprises:
(a) any two or more primer sets selected from the group consisting of: the first primer set, the second primer set, the third primer set, the fourth primer set, the fifth primer set, the sixth primer set, and the seventh primer set;
(b) any three or more primer sets selected from the group consisting of: the first primer set, the second primer set, the third primer set, the fourth primer set, the fifth primer set, the sixth primer set, and the seventh primer set;
(c) any four or more primer sets selected from the group consisting of: the first primer set, the second primer set, the third primer set, the fourth primer set, the fifth primer set, the sixth primer set, and the seventh primer set;
(d) least one primer set selected from the group consisting of: the first primer set, the second primer set, and the third primer set and at least one primer set selected from the group consisting of: the fourth primer set, the fifth primer set, the sixth primer set, and the seventh primer set;
(e) at least one primer set selected from the group consisting of: the fourth primer set and the fifth primer set and at least one primer set selected from the group consisting of: the first primer set, the second primer set, the third primer set, the sixth primer set, and the seventh primer set;
(f) at least one primer set selected from the group consisting of: the sixth primer set and the seventh primer set and at least one primer set selected from the group consisting of: the first primer set, the second primer set, the third primer set the fourth primer set, and the fifth primer set;
(g) at least one primer set selected from the group consisting of: the first primer set, the second primer set, and the third primer set and at least one primer set selected from the group consisting of: the fourth primer set and the fifth primer set and at least one primer set selected from the group consisting of: the sixth primer set, and the seventh primer set;
(h) at least two primer sets selected from the group consisting of: the first primer set, the second primer set, and the third primer set and at least one primer set selected from the group consisting of: the fourth primer set and the fifth primer set and at least one primer set selected from the group consisting of: the sixth primer set, and the seventh primer set;
(i) the first primer set, the fourth primer set, and the sixth primer set; or
(j) comprises the first primer set, the second primer set, the fourth primer set, and the sixth primer set.

3-11. (canceled)

12. The composition or kit of claim 1, wherein
(a) the first primer set, if present, comprises
(i) a first primer comprising a nucleotide sequence having no more than 0, 1, 2, 3, 4, or 5 mismatches from the nucleotide sequence of SEQ ID NO: 126 or an RNA equivalent or a DNA/RNA chimeric thereof, and
(ii) a second primer comprising a nucleotide sequence having no more than 1, 2, 3, 4, or 5 mismatches from the nucleotide sequence of SEQ ID NO: 195, 196, or 197 or an RNA equivalent or a DNA/RNA chimeric thereof;
(b) the second primer set, if present, comprises
(i) a first primer comprising a nucleotide sequence having no more than 0, 1, 2, 3, 4, or 5 mismatches from the nucleotide sequence of SEQ ID NO: 127, 128, 129, 130, or 131 or an RNA equivalent or a DNA/RNA chimeric thereof, and
(ii) a second primer comprising a nucleotide sequence having no more than 1, 2, 3, 4, or 5 mismatches from the nucleotide sequence of SEQ ID NO: 198, 199, 200, 201, 202, 203, 204, 205, 206, 207, 208, or 209 or an RNA equivalent or a DNA/RNA chimeric thereof;
(c) the third primer set, if present, comprises
(i) a first primer comprising a nucleotide sequence having no more than 0, 1, 2, 3, 4, or 5 mismatches from the nucleotide sequence of SEQ ID NO: 132, 133, or 134 or an RNA equivalent or a DNA/RNA chimeric thereof, and
(ii) a second primer comprising a nucleotide sequence having no more than 1, 2, 3, 4, or 5 mismatches from the nucleotide sequence of SEQ ID NO: 210, 211, 212, 213, 214, 215, 216, 217, 218, 219, 220, 221, or 222 or an RNA equivalent or a DNA/RNA chimeric thereof;
(d) the fourth primer set, if present, comprises
(i) a first primer comprising a nucleotide sequence having no more than 0, 1, 2, 3, 4, or 5 mismatches from the nucleotide sequence of SEQ ID NO: 1, 2, 3, 4, or 5 or an RNA equivalent or a DNA/RNA chimeric thereof, and
(ii) a second primer comprising a nucleotide sequence having no more than 1, 2, 3, 4, or 5 mismatches from the nucleotide sequence of SEQ ID NO: 38, 39, 40, 41, 42, or 43 or an RNA equivalent or a DNA/RNA chimeric thereof;
(e) the fifth primer set, if present, comprises
(i) a first primer comprising a nucleotide sequence having no more than 0, 1, 2, 3, 4, or 5 mismatches from the nucleotide sequence of SEQ ID NO: 6, 7, 8, 9, 10, or 11 or an RNA equivalent or a DNA/RNA chimeric thereof, and
(ii) a second primer comprising a nucleotide sequence having no more than 1, 2, 3, 4, or 5 mismatches from the nucleotide sequence of SEQ ID NO: 44, 45, 46, or 47 or an RNA equivalent or a DNA/RNA chimeric thereof;
(f) the sixth primer set, if present, comprises
(i) a first primer comprising a nucleotide sequence having no more than 0, 1, 2, 3, 4, or 5 mismatches from the nucleotide sequence of SEQ ID NO: 72, 73, 74, or 75 or an RNA equivalent or a DNA/RNA chimeric thereof, and
(ii) a second primer comprising a nucleotide sequence having no more than 1, 2, 3, 4, or 5 mismatches from the nucleotide sequence of SEQ ID NO: 104, 105, 106, or 107 or an RNA equivalent or a DNA/RNA chimeric thereof; and
(g) the seventh primer set, if present, comprises
(i) a first primer comprising a nucleotide sequence having no more than 0, 1, 2, 3, 4, or 5 mismatches from the nucleotide sequence of SEQ ID NO: 77 or 78 or an RNA equivalent or a DNA/RNA chimeric thereof, and
(ii) a second primer comprising a nucleotide sequence having no more than 0, 1, 2, 3, 4, or 5 mismatches from the nucleotide sequence of SEQ ID NO: 100, 101, 102, or 103 or an RNA equivalent or a DNA/RNA chimeric thereof.

13. The composition or kit of claim 12, wherein
(a) the first primer set first primer, if present, comprises the nucleotide sequence of SEQ ID NO: 126 or an RNA equivalent or a DNA/RNA chimeric thereof, and the first primer set second primer comprises the nucleotide sequence of SEQ ID NO: 195, 196, or 197 or an RNA equivalent or a DNA/RNA chimeric thereof,
(b) the second primer set first primer, if present, comprises the nucleotide sequence of SEQ ID NO: 127, 128, 129, 130, or 131 or an RNA equivalent ora DNA/RNA chimeric thereof, and the second primer set second primer comprises the nucleotide sequence of SEQ ID NO: 198, 199, 200, 201, 202, 203, 204, 205, 206, 207, 208, or 209 or an RNA equivalent or a DNA/RNA chimeric thereof;
(c) the third primer set first primer, if present, comprises the nucleotide sequence of SEQ ID NO: 132, 133, or 134 or an RNA equivalent or a DNA/RNA chimeric thereof, and the third primer set second primer comprises the nucleotide sequence of SEQ ID NO: 210, 211, 212, 213, 214, 215, 216, 217, 218, 219, 220, 221, or 222 or an RNA equivalent ora DNA/RNA chimeric thereof;
(d) the fourth primer set first primer, if present, comprises the nucleotide sequence of SEQ ID NO: 1, 2, 3, 4, or 5 or an RNA equivalent or a DNA/RNA chimeric thereof, and the fourth primer set second primer comprises the nucleotide sequence SEQ ID NO: 38, 39, 40, 41, 42, or 43 or an RNA equivalent or a DNA/RNA chimeric thereof;
(e) the fifth primer set first primer, if present, comprises the nucleotide sequence of SEQ ID NO: 6, 7, 8, 9, 10, or 11 or an RNA equivalent or a DNA/RNA chimeric thereof, and the fifth primer set second primer comprises the nucleotide of SEQ ID NO: 44, 45, 46, or 47 or an RNA equivalent or a DNA/RNA chimeric thereof;
(f) the sixth primer set first primer, if present, comprises the nucleotide sequence of SEQ ID NO: 72, 73, 74, or 75 or an RNA equivalent or a DNA/RNA chimeric thereof, and the sixth primer set second primer comprises the nucleotide sequence of SEQ ID NO: 104, 105, 106, or 107 or an RNA equivalent or a DNA/RNA chimeric thereof; and
(g) the seventh primer set first primer, if present, comprises the nucleotide sequence of SEQ ID NO: 77 or 78 or an RNA equivalent or a DNA/RNA chimeric thereof, and the seventh primer set second primer comprises the nucleotide sequence of SEQ ID NO: 100, 101, 102, or 103 or an RNA equivalent or a DNA/RNA chimeric thereof.

14. The composition or kit of or claim 1, wherein one or more of the second primers is linked at its 5′ end to an RNA polymerase promoter sequence.

15. The composition or kit of claim 14, wherein
(a) the first primer set second primer, if present, comprises a nucleotide sequence having no more than 0, 1, 2, 3, 4, or 5 mismatches from the nucleotide sequence of SEQ ID NO: 167, 168, or 169 or an RNA equivalent or a DNA/RNA chimeric thereof.
(b) the second primer set second primer, if present, comprises a nucleotide sequence having no more than 0, 1, 2, 3, 4, or 5 mismatches from the nucleotide sequence of SEQ ID NO: 170, 171, 172, 173, 174, 175, 176, 177, 178, 179, 180, or 181 or an RNA equivalent or a DNA/RNA chimeric thereof;
(c) the third primer set second primer, if present, comprises a nucleotide sequence having no more than 0, 1, 2, 3, 4, or 5 mismatches from the nucleotide sequence of SEQ ID NO: 182, 183, 184, 185, 186, 187, 188, 189, 190, 191, 192, 193, or 194 or an RNA equivalent or a DNA/RNA chimeric thereof;
(d) the fourth primer set second primer, if present, comprises a nucleotide sequence having no more than 0, 1, 2, 3, 4, or 5 mismatches from the nucleotide sequence of SEQ ID NO: 28, 29, 30, 31, 32, or 33 or an RNA equivalent or a DNA/RNA chimeric thereof;
(e) the fifth primer set second primer, if present, comprises a nucleotide sequence having no more than 0, 1, 2, 3, 4, or 5 mismatches from the nucleotide sequence of SEQ ID NO: 34, 35, 36, or 37 or an RNA equivalent or a DNA/RNA chimeric thereof;
(f) the sixth primer set second primer, if present, comprises a nucleotide sequence having no more than 0, 1, 2, 3, 4, or 5 mismatches from the nucleotide sequence of SEQ ID NO: 96, 97, 98, or 99 or an RNA equivalent or a DNA/RNA chimeric thereof; and
(g) the seventh primer set second primer, if present, comprises a nucleotide sequence having no more than 0, 1, 2, 3, 4, or 5 mismatches from the nucleotide sequence of SEQ ID NO: 92, 93, 94, or 95 or an RNA equivalent or a DNA/RNA chimeric thereof.

16. The composition or kit of claim 15, wherein
(a) the first primer set second primer, if present, comprises the nucleotide sequence of SEQ ID NO: 167, 168, or 169 or an RNA equivalent or a DNA/RNA chimeric thereof.
(b) the second primer set second primer, if present, comprises the nucleotide sequence of SEQ ID NO: 170, 171, 172, 173, 174, 175, 176, 177, 178, 179, 180, or 181 or an RNA equivalent or a DNA/RNA chimeric thereof;
(c) the third primer set second primer, if present, comprises the nucleotide sequence of SEQ ID NO: 182, 183, 184, 185, 186, 187, 188, 189, 190, 191, 192, 193, or 194 or an RNA equivalent or a DNA/RNA chimeric thereof;
(d) the fourth primer set second primer, if present, comprises the nucleotide sequence of SEQ ID NO: 28, 29, 30, 31, 32, or 33 or an RNA equivalent or a DNA/RNA chimeric thereof;
(e) the fifth primer set second primer, if present, the nucleotide sequence of SEQ ID NO: 34, 35, 36, or 37 or an RNA equivalent or a DNA/RNA chimeric thereof;
(f) the sixth primer set second primer, if present, comprises the nucleotide sequence of SEQ ID NO: 96, 97, 98, or 99 or an RNA equivalent or a DNA/RNA chimeric thereof; and
(g) the seventh primer set second primer, if present, comprises the nucleotide sequence of SEQ ID NO: 92, 93, 94, or 95 or an RNA equivalent or a DNA/RNA chimeric thereof.

17. The composition or kit of or claim 1, wherein the composition or kit comprises:
(a) the first primer set;
(b) the first primer set and the second primer set;
(c) the first primer set and the fourth primer set or the fifth primer set;
(d) the first primer set, the second primer set, and the fourth primer set or the fifth primer set;
(e) the first primer set and the fourth primer set;
(f) the first primer set, the second primer set, and the fourth primer set;
(g) the first primer set and the sixth primer set or the seventh primer set;
(h) the first primer set, the second primer set, and the sixth primer set or the seventh primer set;
(i) the first primer set and the sixth primer set;
(j) the first primer set, the second primer set, and the sixth primer set;
(k) the first primer set, the fourth primer set or the fifth primer set, and the sixth primer set or the seventh primer set;
(l) the first primer set, the second primer set, the fourth primer set or the fifth primer set, and the sixth primer set or the seventh primer set;
(m) the first primer set, the fourth primer set, and the sixth primer set; or
(n) the first primer set, the second primer set, the fourth primer set, and the sixth primer set.

18-24. (canceled)

25. The composition or kit of claim 1, wherein any one or more of the first primer set, the second primer set, the third primer set, the fourth primer set, the fifth primer set, the sixth primer set, and the seventh primer set comprises a first primer and two different second primers.

26. The composition or kit of claim 1, wherein the primer sets are capable of amplifying a target sequence in a transcription-mediated amplification reaction or a biphasic transcription-mediated amplification reaction.

27. (canceled)

28. The composition or kit of claim 1, further comprising a one or more probe oligonucleotides selected from the group consisting of:
(a) a SARS-CoV-2 region 1 probe configured to hybridize to a target sequence contained within an amplicon amplifiable by the first primer set, wherein the SARS-CoV-2 region 1 probe comprises a target hybridizing region 20-30 nucleobases in length, wherein the target hybridizing region is contained within SEQ ID NO: 135, 136, or 137 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 138 or 139 or a DNA equivalent, a DNA/RNA chimeric, and/or a complement thereof;
(b) a SARS-CoV-2 region 2 probe configured to hybridize to a target sequence contained within an amplicon amplifiable by the first primer set, wherein the SARS-CoV-2 region 2 probe comprises a target hybridizing region 14-30 nucleobases in length, wherein the target hybridizing region is contained within SEQ ID NO: 142 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 145 or 146 or a DNA equivalent, a DNA/RNA chimeric, and/or a complement thereof;
(c) a SARS-CoV-2 region 3 probe configured to hybridize to a target sequence contained within an amplicon amplifiable by the first primer set, wherein the SARS-CoV-2 region 3 probe comprises a target hybridizing region 22-35 nucleobases in length, wherein the target hybridizing region is contained within SEQ ID NO: 154 or 158 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 159 or a DNA equivalent, a DNA/RNA chimeric, and/or a complement thereof;
(d) an influenza A region 1 probe configured to hybridize to a target sequence contained within an amplicon amplifiable by the first primer set, wherein the influenza A region 1 probe comprises a target hybridizing region 22-40 nucleobases in length, wherein the target hybridizing region is contained within SEQ ID NO: 12 or 13 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 14 or a DNA equivalent, a DNA/RNA chimeric, and/or a complement thereof;
(e) an influenza A region 2 probe configured to hybridize to a target sequence contained within an amplicon amplifiable by the first primer set, wherein the influenza A region 2 probe comprises a target hybridizing region 19-25 nucleobases in length, wherein the target hybridizing region is contained within SEQ ID NO: 23 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 24 or a DNA equivalent, a DNA/RNA chimeric, and/or a complement thereof;
(f) an influenza B region 1 probe configured to hybridize to a target sequence contained within an amplicon amplifiable by the first primer set, wherein the influenza B region 1 probe comprises a target hybridizing region 23-35 nucleobases in length, wherein the target hybridizing region is contained within SEQ ID NO: 88 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 89 or a DNA equivalent, a DNA/RNA chimeric, and/or a complement thereof; and
(g) an influenza B region 2 probe configured to hybridize to a target sequence contained within an amplicon amplifiable by the first primer set, wherein the influenza B region 2 probe comprises a target hybridizing region 17-30 nucleobases in length, wherein the target hybridizing region is contained within SEQ ID NO: 81 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 82 or a DNA equivalent, a DNA/RNA chimeric, and/or a complement thereof.

29-39. (canceled)

40. The composition or kit of claim 28, wherein
(a) the SARS-CoV-2 region 1 probe, if present, comprises a nucleotide sequence having no more than 0, 1, 2, 3, 4, or 5 mismatches from the nucleotide sequence of SEQ ID NO: 244, 245, 246 or 274, or a DNA equivalent, a DNA/RNA chimeric, and/or a complement thereof;
(b) the SARS-CoV-2 region 2 probe, if present, comprises a nucleotide sequence having no more than 0, 1, 2, 3, 4, or 5 mismatches from the nucleotide sequence of SEQ ID NO: 247, 248, 249, 250, 251, 252, or 258 or a DNA equivalent, a DNA/RNA chimeric, and/or a complement thereof,
(c) the SARS-CoV-2 region 3 probe, if present, comprises a nucleotide sequence having no more than 0, 1, 2, 3, 4, or 5 mismatches from the nucleotide sequence of SEQ ID NO: 253, 254, or 255 or a DNA equivalent, a DNA/RNA chimeric, and/or a complement thereof,
(d) the influenza A region 1 probe, if present, comprises a nucleotide sequence having no more than 0, 1, 2, 3, 4, or 5 mismatches from the nucleotide sequence of SEQ ID NO: 63, 64, 65, 66, 67, or 68 or a DNA equivalent, a DNA/RNA chimeric, and/or a complement thereof,
(e) the influenza A region 2 probe, if present, comprises a nucleotide sequence having no more than 0, 1, 2, 3, 4, or 5 mismatches from the nucleotide sequence of SEQ ID NO: 69, 70, or 71 or a DNA equivalent, a DNA/RNA chimeric, and/or a complement thereof,
(f) the influenza B region 1 probe, if present, comprises a nucleotide sequence having no more than 0, 1, 2, 3, 4, or 5 mismatches from the nucleotide sequence of SEQ ID NO: 120, 121, or 122 or a DNA equivalent, a DNA/RNA chimeric, and/or a complement thereof, and
(g) the influenza A region 2 probe, if present, comprises a nucleotide sequence having no more than 0, 1, 2, 3, 4, or 5 mismatches from the nucleotide sequence of SEQ ID NO: 123, 124, or 125 or a DNA equivalent, a DNA/RNA chimeric, and/or a complement thereof.

41. (canceled)

42. The composition or kit of claim 40, wherein one or more of the probes further comprises a self-complementary sequence joined to the probe target hybridizing region.

43. The composition or kit of claim 42, wherein one or more of the probes comprises a detectable label, a fluorescent label or a fluorescent label and a quencher.

44. The composition or kit of claim 43, wherein one or more of the probes comprises a molecular beacon or a molecular torch.

45. The composition or kit of claim 44, wherein
(a) the SARS-CoV-2 region 1 probe, if present, comprises a nucleotide sequence having no more than 0, 1, 2, 3, 4, or 5 mismatches from the nucleotide sequence of SEQ ID NO: 232, 233, 234, 266, 267, 268, 269, 270, 271, 272, 273, 274, or 275, or a DNA equivalent, a DNA/RNA chimeric, and/or a complement thereof.
(b) the SARS-CoV-2 region 2 probe, if present, comprises a nucleotide sequence having no more than 0, 1, 2, 3, 4, or 5 mismatches from the nucleotide sequence of SEQ ID NO: 235, 236, 237, 238, 239, 240, or 257 or a DNA equivalent, a DNA/RNA chimeric, and/or a complement thereof;
(c) the SARS-CoV-2 region 3 probe, if present, comprises a nucleotide sequence having no more than 0, 1, 2, 3, 4, or 5 mismatches from the nucleotide sequence of SEQ ID NO: 241, 242, or 243 or a DNA equivalent, a DNA/RNA chimeric, and/or a complement thereof;
(d) the influenza A region 1 probe, if present, comprises a nucleotide sequence having no more than 0, 1, 2, 3, 4, or 5 mismatches from the nucleotide sequence of SEQ ID NO: 54, 55, 56, 57, 58, or 59 or a DNA equivalent, a DNA/RNA chimeric, and/or a complement thereof;
(e) the influenza A region 2 probe, if present, comprises a nucleotide sequence having no more than 0, 1, 2, 3, 4, or 5 mismatches from the nucleotide sequence of SEQ ID NO: 61, or 62 or a DNA equivalent, a DNA/RNA chimeric, and/or a complement thereof;
(f) the influenza B region 1 probe, if present, comprises a nucleotide sequence having no more than 0, 1, 2, 3, 4, or 5 mismatches from the nucleotide sequence of SEQ ID NO: 114, 115, or 116 or a DNA equivalent, a DNA/RNA chimeric, and/or a complement thereof; and
(g) the influenza B region 2 probe, if present, comprises a nucleotide sequence having no more than 0, 1, 2, 3, 4, or 5 mismatches from the nucleotide sequence of SEQ ID NO: 117, 118, or 119 or a DNA equivalent, a DNA/RNA chimeric, and/or a complement thereof.

46. (canceled)

47. The composition or kit of claim 28, wherein the composition or kit comprises
(a) the SARS-CoV-2 region 1 probe;
(b) the SARS-CoV-2 region 1 probe and the SARS-CoV-2 region 2 probe;
(c) the SARS-CoV-2 region 1 probe and the influenza A region 1 probe or the influenza A region 2 probe;
(d) the SARS-CoV-2 region 1 probe, the SARS-CoV-2 region 2 probe, influenza A region 1 probe or the influenza A region 2 probe;
(e) the SARS-CoV-2 region 1 probe and the influenza A region 1 probe;
(f) the SARS-CoV-2 region 1 probe, the SARS-CoV-2 region 2 probe, influenza A region 1 probe;
(g) the SARS-CoV-2 region 1 probe and the influenza B region 1 probe or the influenza B region 2 probe;
(h) the SARS-CoV-2 region 1 probe, the SARS-CoV-2 region 2 probe, and the influenza B region 1 probe or the influenza B region 2 probe;
(i) the SARS-CoV-2 region 1 probe and the influenza B region 1 probe;
(j) the SARS-CoV-2 region 1 probe, the SARS-CoV-2 region 2 probe, and the influenza B region 1 probe;
(k) the SARS-CoV-2 region 1 probe, the influenza A region 1 probe or the influenza A region 2 probe, and the influenza B region 1 probe or the influenza B region 2 probe;
(l) the SARS-CoV-2 region 1 probe, the SARS-CoV-2 region 2 probe, the influenza A region 1 probe or the influenza A region 2 probe, and the influenza B region 1 probe or the influenza B region 2 probe;
(m) the SARS-CoV-2 region 1 probe, the influenza A region 1 probe, and the influenza B region 1 probe; or
(n) the SARS-CoV-2 region 1 probe, the SARS-CoV-2 region 2 probe, the influenza A region 1 probe, and the influenza B region 1 probe.

48-57. (canceled)

58. The composition or kit of claim 47, wherein the composition comprises at least one SARS-CoV-2 probe and at least one Influenza A probe and/or at least one Influenza B probe, wherein the at least SARS-CoV-2 probe, the at least one Influenza A probe, and the at least one Influenza B probe are distinguishable from each other.

59. The composition or kit of claim 28, wherein the composition or kit comprises
(a) SEQ ID NO: 126, at least one of SEQ ID NO: 167 and SEQ ID NO: 169, and at least one of SEQ ID NO: 234, 266, 267, 268, 269, 270, 271, 272, 273, 274, and 275;
(b) SEQ ID NO: 126, at least one of SEQ ID NO: 167 and SEQ ID NO: 169, at least one of SEQ ID NO: 234, 266, 267, 268, 269, 270, 271, 272, 273, 274, and 275, SEQ ID NO: 127, SEQ ID NO: 172, and SEQ ID NO: 235;
(c) SEQ ID NO: 126, at least one of SEQ ID NO: 167 and SEQ ID NO: 169, at least one of SEQ ID NO: 234, 266, 267, 268, 269, 270, 271, 272, 273, 274, and 275, SEQ ID NO: 1, SEQ ID NO: 28, and at least one of SEQ ID NO: 54 and SEQ ID NO: 55,
(d) SEQ ID NO: 126, at least one of SEQ ID NO: 167 and SEQ ID NO: 169, at least one of SEQ ID NO: 234, 266, 267, 268, 269, 270, 271, 272, 273, 274, and 275, SEQ ID NO: 72, SEQ ID NO: 98, and SEQ ID NO: 116;
(e) SEQ ID NO: 126, at least one of SEQ ID NO: 167 and SEQ ID NO: 169, at least one of SEQ ID NO: 234, 266, 267, 268, 269, 270, 271, 272, 273, 274, and 275, SEQ ID NO: 127, SEQ ID NO: 172, SEQ ID NO: 235, SEQ ID NO: 1, SEQ ID NO: 28, at least one of SEQ ID NO: 54 and SEQ ID NO: 55, SEQ ID NO: 72, SEQ ID NO: 98, and SEQ ID NO: 116; or
(f) SEQ ID NO: 126, at least one of SEQ ID NO: 167 and SEQ ID NO: 169, SEQ ID NO: 266, SEQ ID NO: 127, SEQ ID NO: 172, SEQ ID NO: 235, SEQ ID NO: 1, SEQ ID NO: 28, at least one of SEQ ID NO: 54 and SEQ ID NO: 55, SEQ ID NO: 72, SEQ ID NO: 98, and SEQ ID NO: 116.

60-62. (canceled)

63. The composition of kit of claim 1, wherein the composition or kit further comprises one or more target capture oligonucleotides (TCO) selected from the group consisting of:
(a) at least one SARS-CoV-2 target capture oligonucleotide comprising a target hybridizing region 22-30 nucleobases in length, wherein the target hybridizing region comprises a nucleotide sequence differing by no more than 0, 1, 2, 3, 4, or 5 nucleotides from the nucleotide sequence of SEQ ID NO: 228, 229, 230, 231, or 256 or an RNA equivalent, a DNA/RNA chimeric, and/or complement thereof;
(b) an influenza A target capture comprising a target hybridizing region 26-30 nucleobases in length, wherein the target hybridizing region comprises a nucleotide sequence differing by no more than 0, 1, 2, 3, 4, or 5 nucleotides from the nucleotide sequence of SEQ ID NO: 51, 52, or 53 or an RNA equivalent, a DNA/RNA chimeric, and/or complement thereof; and
(c) an influenza B target capture oligonucleotide comprising a target hybridizing region 25-30 nucleobases in length, wherein the target hybridizing region comprises a nucleotide sequence differing by no more than 0, 1, 2, 3, 4, or 5 nucleotides from the nucleotide sequence of SEQ ID NO: 111, 112, or 113 or an RNA equivalent, a DNA/RNA chimeric, and/or complement thereof;
wherein the one or more TCOs are independently linked to TₙAₘ sequences, wherein n in an integer from 0 to 3 and m in an integer from 14 to 50.

64-66. (canceled)

67. The composition or kit of claim 1, wherein at least one of the primers comprises one or more modified nucleotides.

68. (canceled)

69. A formulation for determining the presence or absence of SARS-CoV-2, influenza A, and/or influenza B in a sample, said formulation comprising one or more of the following primer sets:
(a) a first primer set comprising first and second SARS-CoV-2 region 1 primers capable of amplifying a target region of a SARS-CoV-2 target nucleic acid wherein,
(i) the SARS-CoV-2 region 1 first primer comprises a nucleotide sequence having no more than 0, 1, 2, 3, 4, or 5 mismatches from the nucleotide sequence of SEQ ID NO: 126 or an RNA equivalent or a DNA/RNA chimeric thereof, and
(ii) the SARS-CoV-2 region 1 second primer comprises a target hybridizing region 18-35 nucleobases in length, wherein the target hybridizing region is contained within SEQ ID NO: 140 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 141 or an RNA equivalent or a DNA/RNA chimeric thereof;
(b) a second primer set comprising first and second SARS-CoV-2 region 2 primers capable of amplifying a target region of a SARS-CoV-2 target nucleic acid wherein,
(i) the SARS-CoV-2 region 2 first primer comprises a target hybridizing region 16-30 nucleobases in length, wherein the target hybridizing region is contained within SEQ ID NO: 143 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 144 or an RNA equivalent or a DNA/RNA chimeric thereof, and
(ii) the SARS-CoV-2 region 2 second primer comprises a target hybridizing region 11-35 nucleobases in length, wherein the target hybridizing region is contained within SEQ ID NO: 147, 148, or 149 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 150, 151, or 152 or an RNA equivalent or a DNA/RNA chimeric thereof;
(c) a third primer set comprising first and second SARS-CoV-2 region 3 primers capable of amplifying a target region of a SARS-CoV-2 target nucleic acid wherein,
(i) the SARS-CoV-2 region 3 first primer comprises target hybridizing region 18-30 nucleobases in length, wherein the target hybridizing region is contained within SEQ ID NO: 155 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 156 or 157 or an RNA equivalent or a DNA/RNA chimeric thereof, and
(ii) the SARS-CoV-2 region 3 second primer comprises a target hybridizing region 13-40 nucleobases in length, wherein the target hybridizing region is contained within SEQ ID NO: 160, 161, or 162 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 163, 164, 165, or 166 or an RNA equivalent or a DNA/RNA chimeric thereof;
(d) a fourth primer set comprising first and second Influenza A region 1 primers capable of amplifying a target region of an Influenza A target nucleic acid,
(i) the influenza A region 1 first primer comprises a target hybridizing region 15-30 nucleobases in length, wherein the target hybridizing region is contained within SEQ ID NO:15 or an RNA equivalent or a DNA/RNA chimeric thereof and comprises a nucleotide sequence differing by no more than 0, 1, 2, 3, 4, or 5 nucleotides from the nucleotide sequence of SEQ ID NO:16 or SEQ ID NO:17 an RNA equivalent or a DNA/RNA chimeric thereof, and
(ii) the influenza A region 1 second primer comprises a target hybridizing region 15-30 nucleobases in length, wherein the target hybridizing region is contained within SEQ ID NO:18 an RNA equivalent or a DNA/RNA chimeric thereof and comprises a nucleotide sequence differing by no more than 0, 1, 2, 3, 4, or 5 nucleotides from the nucleotide sequence of SEQ ID NO:19 or SEQ ID NO:20 or an RNA equivalent or a DNA/RNA chimeric thereof;
(e) a fifth primer set comprising first and second Influenza A region 2 primers capable of amplifying a target region of an Influenza A target nucleic acid,
(i) the influenza A region 2 first primer comprises a target hybridizing region 15-30 nucleobases in length, wherein the target hybridizing region is contained within SEQ ID NO: 25 an RNA equivalent or a DNA/RNA chimeric thereof and comprises a nucleotide sequence differing by no more than 0, 1, 2, 3, 4, or 5 nucleotides from the nucleotide sequence of SEQ ID NO: 26 or SEQ ID NO: 27 or an RNA equivalent or a DNA/RNA chimeric thereof, and
(ii) the influenza A region 2 second primer comprises a target hybridizing region 15-30 nucleobases in length, wherein the target hybridizing region is contained within SEQ ID NO: 21 or an RNA equivalent or a DNA/RNA chimeric thereof and comprises a nucleotide sequence differing by no more than 0, 1, 2, 3, 4, or 5 nucleotides from the nucleotide sequence of SEQ ID NO:22 or an RNA equivalent or a DNA/RNA chimeric thereof;
(f) a sixth primer set comprising first and second Influenza B region 1 primers capable of amplifying a target region of an Influenza B target nucleic acid,
(i) the influenza B region 1 first primer comprises a target hybridizing region 20-30 nucleobases in length, wherein the target hybridizing region is contained within SEQ ID NO: 90 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 91 or an RNA equivalent or a DNA/RNA chimeric thereof, and
(ii) the influenza B region 1 second primer comprises a target hybridizing region 17-30 nucleobases in length, wherein the target hybridizing region is contained within SEQ ID NO: 85 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 86 or SEQ ID NO: 87 or an RNA equivalent or a DNA/RNA chimeric thereof; and
(g) a seventh primer set comprising first and second Influenza B region 2 primers capable of amplifying a target region of an Influenza B target nucleic acid,
(i) the influenza B region 2 first primer comprises a target hybridizing region 13-30 nucleobases in length, wherein the target hybridizing region is contained within SEQ ID NO: 83 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 84 or an RNA equivalent or a DNA/RNA chimeric thereof, and
(ii) the influenza B region 2 second primer comprises a target hybridizing region 17-30 nucleobases in length, wherein the target hybridizing region is contained within SEQ ID NO: 79 or an RNA equivalent or a DNA/RNA chimeric thereof and comprises a nucleotide sequence differing by no more than 0, 1, 2, 3, 4, or 5 nucleotides from the nucleotide sequence of SEQ ID NO: 80 or an RNA equivalent or a DNA/RNA chimeric thereof.

70. The formulation of claim 69, further comprising one or more probes selected from the group consisting of:
(a) a SARS-CoV-2 region 1 probe configured to hybridize to a target sequence contained within an amplicon amplifiable by the first primer set, wherein the SARS-CoV-2 region 1 probe comprises a target hybridizing region 20-30 nucleobases in length, wherein the target hybridizing region is contained within SEQ ID NO: 135, 136, or 137 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 138 or 139 or a DNA equivalent, a DNA/RNA chimeric, and/or a complement thereof;
(b) a SARS-CoV-2 region 2 probe configured to hybridize to a target sequence contained within an amplicon amplifiable by the first primer set, wherein the SARS-CoV-2 region 2 probe comprises a target hybridizing region 14-30 nucleobases in length, wherein the target hybridizing region is contained within SEQ ID NO: 142 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 145 or 146 or a DNA equivalent, a DNA/RNA chimeric, and/or a complement thereof;
(c) a SARS-CoV-2 region 3 probe configured to hybridize to a target sequence contained within an amplicon amplifiable by the first primer set, wherein the SARS-CoV-2 region 3 probe comprises a target hybridizing region 22-35 nucleobases in length, wherein the target hybridizing region is contained within SEQ ID NO: 154 or 158 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 159 or a DNA equivalent, a DNA/RNA chimeric, and/or a complement thereof;
(d) an influenza A region 1 probe configured to hybridize to a target sequence contained within an amplicon amplifiable by the first primer set, wherein the influenza A region 1 probe comprises a target hybridizing region 22-40 nucleobases in length, wherein the target hybridizing region is contained within SEQ ID NO: 12 or 13 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 14 or a DNA equivalent, a DNA/RNA chimeric, and/or a complement thereof;
(e) an influenza A region 2 probe configured to hybridize to a target sequence contained within an amplicon amplifiable by the first primer set, wherein the influenza A region 2 probe comprises a target hybridizing region 19-25 nucleobases in length, wherein the target hybridizing region is contained within SEQ ID NO: 23 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 24 or a DNA equivalent, a DNA/RNA chimeric, and/or a complement thereof;
(f) an influenza B region 1 probe configured to hybridize to a target sequence contained within an amplicon amplifiable by the first primer set, wherein the influenza B region 1 probe comprises a target hybridizing region 23-35 nucleobases in length, wherein the target hybridizing region is contained within SEQ ID NO: 88 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 89 or a DNA equivalent, a DNA/RNA chimeric, and/or a complement thereof; and
(g) an influenza B region 2 probe configured to hybridize to a target sequence contained within an amplicon amplifiable by the first primer set, wherein the influenza B region 2 probe comprises a target hybridizing region 17-30 nucleobases in length, wherein the target hybridizing region is contained within SEQ ID NO: 81 or a DNA equivalent, a DNA/RNA chimeric, and/or a complement thereof and comprises a nucleotide sequence differing by no more than 0, 1, 2, 3, 4, or 5 nucleotides from the nucleotide sequence of SEQ ID NO: 82 or a DNA equivalent, a DNA/RNA chimeric, and/or a complement thereof.

71. (canceled)

72. The formulation of claim 69, wherein the one or more of the primer sets are provided in one or more aqueous solutions or one or more dried or lyophilized powders or cakes; or wherein the formulation comprises two or more primers sets provided in an aqueous solution of a dried or lyophilized powder or cake.

73. (canceled)

74. A method for amplifying a target sequence of SARS-CoV-2, influenza A, and/or influenza B present in a sample, the method comprising:
(a) contacting the sample one or more primer sets of claim 1; and
(b) exposing the sample to conditions sufficient to amplify the target region thereby producing one or more amplification products.

75-80. (canceled)


SEQUENCE LISTING (275 sequences)

SEQ ID NO: 1 - DNA, 23 bp, Influenza A
ggtcttgtctttagccattccat

SEQ ID NO: 2 - DNA, 24 bp, Influenza A
ccattccatgagagcctcaagatc

SEQ ID NO: 3 - DNA, 27 bp, Influenza A
ctttagccattccatgagagcctcaag

SEQ ID NO: 4 - DNA, 29 bp, Influenza A
cttgtctttagccattccatgagagcctc

SEQ ID NO: 5 - DNA, 24 bp, Influenza A
ggtcttgtctttagccattccatg

SEQ ID NO: 6 - DNA, 26 bp, Influenza A
cccattgagggcattttggacaaagc

SEQ ID NO: 7 - DNA, 23 bp, Influenza A
cattgagggcattttggacaaag

SEQ ID NO: 8 - DNA, 22 bp, Influenza A
ccattgagggcattttggacaa

SEQ ID NO: 9 - DNA, 26 bp, Influenza A
cattcccattgagggcattttggaca

SEQ ID NO: 10 - DNA, 27 bp, Influenza A
ctccattcccattgagggcattttgga

SEQ ID NO: 11 - DNA, 23 bp, Influenza A
gggtctccattcccattgagggc

SEQ ID NO: 12 - DNA, 65 bp, Influenza A
  misc_feature: U can be T or U
ggucuugucuuuagccauuccaugagagccucaagaucuguguuyuuyccwgcaaakacacuuuc

SEQ ID NO: 13 - DNA, 35 bp, Influenza A
ucaagaucuguguuyuuyccwgcaaakacacuuuc

SEQ ID NO: 14 - DNA, 22 bp, Influenza A
cuguguuyuuyccwgcaaakac

SEQ ID NO: 15 - DNA, 38 bp, Influenza A
ggtcttgtctttagccattccatgagagcctcaagatc

SEQ ID NO: 16 - DNA, 15 bp, Influenza A
ctttagccattccat

SEQ ID NO: 17 - DNA, 18 bp, Influenza A
ccattccatgagagcctc

SEQ ID NO: 18 - DNA, 37 bp, Influenza A
cgtcaggccccctcaaagccgagatcgcacaaagact

SEQ ID NO: 19 - DNA, 20 bp, Influenza A
caggccccctcaaagccgag

SEQ ID NO: 20 - DNA, 14 bp, Influenza A
caaagccgagatcg

SEQ ID NO: 21 - DNA, 35 bp, Influenza A
gratttgtgttcacgctcaccgtgcccagtgagcg

SEQ ID NO: 22 - DNA, 23 bp, Influenza A
gtgttcacgctcaccgtgcccag

SEQ ID NO: 23 - DNA, 25 bp, Influenza A
agcgucuacgcugcaguccucgcuc

SEQ ID NO: 24 - DNA, 19 bp, Influenza A
cgucuacgcugcaguccuc

SEQ ID NO: 25 - DNA, 37 bp, Influenza A
gggtcyccattyccattkagggcattttggataaagc

SEQ ID NO: 26 - DNA, 18 bp, Influenza A
cattkagggcattttgga

SEQ ID NO: 27 - DNA, 16 bp, Influenza A
cattyccattkagggc

SEQ ID NO: 28 - DNA, 54 bp, Artificial Sequence
  Synthetic oligonucleotide
aatttaatacgactcactatagggagaatcaggccccctcaaagccgagatcgc

SEQ ID NO: 29 - DNA, 53 bp, Artificial Sequence
  Synthetic oligonucleotide
aatttaatacgactcactatagggagacatcaggccccctcaaagccgagatc

SEQ ID NO: 30 - DNA, 47 bp, Artificial Sequence
  Synthetic oligonucleotide
aatttaatacgactcactatagggagacaggccccctcaaagccgag

SEQ ID NO: 31 - DNA, 51 bp, Artificial Sequence
  Synthetic oligonucleotide
aatttaatacgactcactatagggagacaggccccctcaaagccgagatcg

SEQ ID NO: 32 - DNA, 52 bp, Artificial Sequence
  Synthetic oligonucleotide
aatttaatacgactcactatagggagaccctcaaagccgagatcgcgcagag

SEQ ID NO: 33 - DNA, 51 bp, Artificial Sequence
  Synthetic oligonucleotide
aatttaatacgactcactatagggagacaaagccgagatcgcgcagagact

SEQ ID NO: 34 - DNA, 58 bp, Artificial Sequence
  Synthetic oligonucleotide
aatttaatacgactcactatagggagagggtttgtgttcacgctcaccgtgcccagtg

SEQ ID NO: 35 - DNA, 54 bp, Artificial Sequence
  Synthetic oligonucleotide
aatttaatacgactcactatagggagagtttgtgttcacgctcaccgtgcccag

SEQ ID NO: 36 - DNA, 57 bp, Artificial Sequence
  Synthetic oligonucleotide
aatttaatacgactcactatagggagagtttgtgttcacgctcaccgtgcccagtga

SEQ ID NO: 37 - DNA, 56 bp, Artificial Sequence
  Synthetic oligonucleotide
aatttaatacgactcactatagggagagtgttcacgctcaccgtgcccagtgagcg

SEQ ID NO: 38 - DNA, 27 bp, Influenza A
atcaggccccctcaaagccgagatcgc

SEQ ID NO: 39 - DNA, 26 bp, Influenza A
catcaggccccctcaaagccgagatc

SEQ ID NO: 40 - DNA, 20 bp, Influenza A
caggccccctcaaagccgag

SEQ ID NO: 41 - DNA, 24 bp, Influenza A
caggccccctcaaagccgagatcg

SEQ ID NO: 42 - DNA, 25 bp, Influenza A
ccctcaaagccgagatcgcgcagag

SEQ ID NO: 43 - DNA, 24 bp, Influenza A
caaagccgagatcgcgcagagact

SEQ ID NO: 44 - DNA, 31 bp, Influenza A
gggtttgtgttcacgctcaccgtgcccagtg

SEQ ID NO: 45 - DNA, 27 bp, Influenza A
gtttgtgttcacgctcaccgtgcccag

SEQ ID NO: 46 - DNA, 30 bp, Influenza A
gtttgtgttcacgctcaccgtgcccagtga

SEQ ID NO: 47 - DNA, 29 bp, Influenza A
gtgttcacgctcaccgtgcccagtgagcg

SEQ ID NO: 48 - DNA, 59 bp, Artificial Sequence
  Synthetic oligonucleotide
cucaccgugcccagugagcgaggacutttaaaaaaaaaaaaaaaaaaaaaaaaaaaaaa

SEQ ID NO: 49 - DNA, 62 bp, Artificial Sequence
  Synthetic oligonucleotide
ggugcacuugccaguugcaugggccucautttaaaaaaaaaaaaaaaaaaaaaaaaaaaaaa

SEQ ID NO: 50 - DNA, 63 bp, Artificial Sequence
  Synthetic oligonucleotide
gcacuugccaguugcaugggccucauauactttaaaaaaaaaaaaaaaaaaaaaaaaaaaaaa

SEQ ID NO: 51 - DNA, 26 bp, Influenza A
cucaccgugcccagugagcgaggacu

SEQ ID NO: 52 - DNA, 29 bp, Influenza A
ggugcacuugccaguugcaugggccucau

SEQ ID NO: 53 - DNA, 30 bp, Influenza A
gcacuugccaguugcaugggccucauauac

SEQ ID NO: 54 - DNA, 36 bp, Artificial Sequence
  Synthetic oligonucleotide
gcaagaucuguguucuuuccugcaaagacaucuugc

SEQ ID NO: 55 - DNA, 36 bp, Artificial Sequence
  Synthetic oligonucleotide
gcaagaucuguguuuuucccagcaaagacaucuugc

SEQ ID NO: 56 - DNA, 36 bp, Artificial Sequence
  Synthetic oligonucleotide
gaucuguguuuuucccagcaaagacaucuucagauc

SEQ ID NO: 57 - DNA, 32 bp, Artificial Sequence
  Synthetic oligonucleotide
ucuguguuuuucccagcaaagacaucuacaga

SEQ ID NO: 58 - DNA, 31 bp, Artificial Sequence
  Synthetic oligonucleotide
gaucuguguuuuucccagcaaagacaagauc

SEQ ID NO: 59 - DNA, 27 bp, Artificial Sequence
  Synthetic oligonucleotide
cuguguuuuucccagcaaagaccacag

SEQ ID NO: 60 - DNA, 27 bp, Artificial Sequence
  Synthetic oligonucleotide
cgucuacgcugcaguccucgcucgacg

SEQ ID NO: 61 - DNA, 25 bp, Artificial Sequence
  Synthetic oligonucleotide
gcgucuacgcugcaguccucgacgc

SEQ ID NO: 62 - DNA, 25 bp, Artificial Sequence
  Synthetic oligonucleotide
agcgucuacgcugcaguccuccgcu

SEQ ID NO: 63 - DNA, 30 bp, Influenza A
gcaagaucuguguucuuuccugcaaagaca

SEQ ID NO: 64 - DNA, 30 bp, Influenza A
gcaagaucuguguuuuucccagcaaagaca

SEQ ID NO: 65 - DNA, 31 bp, Influenza A
gaucuguguuuuucccagcaaagacaucuuc

SEQ ID NO: 66 - DNA, 27 bp, Influenza A
ucuguguuuuucccagcaaagacaucu

SEQ ID NO: 67 - DNA, 26 bp, Influenza A
gaucuguguuuuucccagcaaagaca

SEQ ID NO: 68 - DNA, 22 bp, Influenza A
cuguguuuuucccagcaaagac

SEQ ID NO: 69 - DNA, 23 bp, Influenza A
cgucuacgcugcaguccucgcuc

SEQ ID NO: 70 - DNA, 21 bp, Influenza A
gcgucuacgcugcaguccucg

SEQ ID NO: 71 - DNA, 21 bp, Influenza A
agcgucuacgcugcaguccuc

SEQ ID NO: 72 - DNA, 25 bp, Influenza B
gcagagtctaggtcaaattctttcc

SEQ ID NO: 73 - DNA, 25 bp, Influenza B
gcagagtctaggtcaaattcttttc

SEQ ID NO: 74 - DNA, 28 bp, Influenza B
caaggcagagtctaggtcaaattctttc

SEQ ID NO: 75 - DNA, 29 bp, Influenza B
cattccaaggcagagtctaggtcaaattc

SEQ ID NO: 76 - skipped

SEQ ID NO: 77 - DNA, 19 bp, Influenza B
cggtgctcttgaccaaatt

SEQ ID NO: 78 - DNA, 24 bp, Influenza B
ctggtgataatcggtgctcttgac

SEQ ID NO: 79 - DNA, 35 bp, Influenza B
gaaggacattcaaagccaattcgagcagctgaaac

SEQ ID NO: 80 - DNA, 16 bp, Influenza B
caaagccaattcgagc

SEQ ID NO: 81 - DNA, 32 bp, Influenza B
caaauugsgauaagacucccaccgcaguuuca

SEQ ID NO: 82 - DNA, 17 bp, Influenza B
sgauaagacucccaccg

SEQ ID NO: 83 - DNA, 30 bp, Influenza B
ctggtgatagtcggtgctcttgaccaaatt

SEQ ID NO: 84 - DNA, 13 bp, Influenza B
cggtgctcttgac

SEQ ID NO: 85 - DNA, 38 bp, Influenza B
cctgctttcattgacagaagatggagaaggcaaagcag

SEQ ID NO: 86 - DNA, 21 bp, Influenza B
cattgacagaagatggagaag

SEQ ID NO: 87 - DNA, 17 bp, Influenza B
cagaagatggagaaggc

SEQ ID NO: 88 - DNA, 31 bp, Influenza B
caccgaaccaacaguguaauuuuucugcuag

SEQ ID NO: 89 - DNA, 23 bp, Influenza B
gaaccaacaguguaauuuuucug

SEQ ID NO: 90 - DNA, 34 bp, Influenza B
cattccaaggcagagtctaggtcaaattctttyc

SEQ ID NO: 91 - DNA, 20 bp, Influenza B
gcagagtctaggtcaaattc

SEQ ID NO: 92 - DNA, 53 bp, Artificial Sequence
  Synthetic oligonucleotide
aatttaatacgactcactatagggagagaaggacattcaaagccaattcgagc

SEQ ID NO: 93 - DNA, 52 bp, Artificial Sequence
  Synthetic oligonucleotide
aatttaatacgactcactatagggagaggacattcaaagccaattcgagcag

SEQ ID NO: 94 - DNA, 52 bp, Artificial Sequence
  Synthetic oligonucleotide
aatttaatacgactcactatagggagacattcaaagccaattcgagcagctg

SEQ ID NO: 95 - DNA, 52 bp, Artificial Sequence
  Synthetic oligonucleotide
aatttaatacgactcactatagggagacaaagccaattcgagcagctgaaac

SEQ ID NO: 96 - DNA, 56 bp, Artificial Sequence
  Synthetic oligonucleotide
aatttaatacgactcactatagggagacctgctttcattgacagaagatggagaag

SEQ ID NO: 97 - DNA, 54 bp, Artificial Sequence
  Synthetic oligonucleotide
aatttaatacgactcactatagggagactttcattgacagaagatggagaaggc

SEQ ID NO: 98 - DNA, 55 bp, Artificial Sequence
  Synthetic oligonucleotide
aatttaatacgactcactatagggagacattgacagaagatggagaaggcaaagc

SEQ ID NO: 99 - DNA, 51 bp, Artificial Sequence
  Synthetic oligonucleotide
aatttaatacgactcactatagggagacagaagatggagaaggcaaagcag

SEQ ID NO: 100 - DNA, 26 bp, Influenza B
gaaggacattcaaagccaattcgagc

SEQ ID NO: 101 - DNA, 25 bp, Influenza B
ggacattcaaagccaattcgagcag

SEQ ID NO: 102 - DNA, 25 bp, Influenza B
cattcaaagccaattcgagcagctg

SEQ ID NO: 103 - DNA, 25 bp, Influenza B
caaagccaattcgagcagctgaaac

SEQ ID NO: 104 - DNA, 29 bp, Influenza B
cctgctttcattgacagaagatggagaag

SEQ ID NO: 105 - DNA, 27 bp, Influenza B
ctttcattgacagaagatggagaaggc

SEQ ID NO: 106 - DNA, 28 bp, Influenza B
cattgacagaagatggagaaggcaaagc

SEQ ID NO: 107 - DNA, 24 bp, Influenza B
cagaagatggagaaggcaaagcag

SEQ ID NO: 108 - DNA, 58 bp, Artificial Sequence
  Synthetic oligonucleotide
cugauauacagaaagcacuaauuggtttaaaaaaaaaaaaaaaaaaaaaaaaaaaaaa

SEQ ID NO: 109 - DNA, 59 bp, Artificial Sequence
  Synthetic oligonucleotide
guggaggaugaagaagauggccaucgtttaaaaaaaaaaaaaaaaaaaaaaaaaaaaaa

SEQ ID NO: 110 - DNA, 60 bp, Artificial Sequence
  Synthetic oligonucleotide
gaggaugaagaagauggccaucggauctttaaaaaaaaaaaaaaaaaaaaaaaaaaaaaa

SEQ ID NO: 111 - DNA, 25 bp, Influenza B
cugauauacagaaagcacuaauugg

SEQ ID NO: 112 - DNA, 26 bp, Influenza B
guggaggaugaagaagauggccaucg

SEQ ID NO: 113 - DNA, 27 bp, Influenza B
gaggaugaagaagauggccaucggauc

SEQ ID NO: 114 - DNA, 33 bp, Artificial Sequence
  Synthetic oligonucleotide
cgaaccaacaguguaauuuuucugcuagguucg

SEQ ID NO: 115 - DNA, 29 bp, Artificial Sequence
  Synthetic oligonucleotide
gaaccaacaguguaauuuuucugcgguuc

SEQ ID NO: 116 - DNA, 33 bp, Artificial Sequence
  Synthetic oligonucleotide
caccgaaccaacaguguaauuuuucugccggug

SEQ ID NO: 117 - DNA, 29 bp, Artificial Sequence
  Synthetic oligonucleotide
cgauaagacucccaccgcaguuucuaucg

SEQ ID NO: 118 - DNA, 25 bp, Artificial Sequence
  Synthetic oligonucleotide
ggauaagacucccaccgcaguaucc

SEQ ID NO: 119 - DNA, 27 bp, Artificial Sequence
  Synthetic oligonucleotide
cauugggauaagacucccaccgcaaug

SEQ ID NO: 120 - DNA, 28 bp, Influenza B
cgaaccaacaguguaauuuuucugcuag

SEQ ID NO: 121 - DNA, 24 bp, Influenza B
gaaccaacaguguaauuuuucugc

SEQ ID NO: 122 - DNA, 28 bp, Influenza B
caccgaaccaacaguguaauuuuucugc

SEQ ID NO: 123 - DNA, 24 bp, Influenza B
cgauaagacucccaccgcaguuuc

SEQ ID NO: 124 - DNA, 20 bp, Influenza B
ggauaagacucccaccgcag

SEQ ID NO: 125 - DNA, 22 bp, Influenza B
cauugggauaagacucccaccg

SEQ ID NO: 126 - DNA, 25 bp, SARS-CoV-2
gcttgatggctttatgggtagaatt

SEQ ID NO: 127 - DNA, 19 bp, SARS-CoV-2
gctcggtatatgagatctc

SEQ ID NO: 128 - DNA, 20 bp, SARS-CoV-2
gctgctcggtatatgagatc

SEQ ID NO: 129 - DNA, 19 bp, SARS-CoV-2
ctgctcggtatatgagatc

SEQ ID NO: 130 - DNA, 19 bp, SARS-CoV-2
gctcggtatatgagatctc

SEQ ID NO: 131 - DNA, 20 bp, SARS-CoV-2
ctcggtatatgagatctctc

SEQ ID NO: 132 - DNA, 21 bp, SARS-CoV-2
cagggtgctgtagacataaac

SEQ ID NO: 133 - DNA, 22 bp, SARS-CoV-2
ggtgctgtagacataaacaagc

SEQ ID NO: 134 - DNA, 20 bp, SARS-CoV-2
ctgtagacataaacaagctt

SEQ ID NO: 135 - DNA, 53 bp, SARS-CoV-2
  misc_feature: U can be T or U
gcuugauggcuuuauggguagaauucgaucugucuauccaguugcgucaccaa

SEQ ID NO: 136 - DNA, 26 bp, SARS-CoV-2
cgaucugucuauccaguugcgucacc

SEQ ID NO: 137 - DNA, 28 bp, SARS-CoV-2
cgaucugucuauccaguugcgucaccaa

SEQ ID NO: 138 - DNA, 22 bp, SARS-CoV-2
gaucugucuauccaguugcguc

SEQ ID NO: 139 - DNA, 20 bp, SARS-CoV-2
ucugucuauccaguugcguc

SEQ ID NO: 140 - DNA, 33 bp, SARS-CoV-2
gagagttgaaaggcacatttggttgcattcatt

SEQ ID NO: 141 - DNA, 18 bp, SARS-CoV-2
gagttgaaaggcacattt

SEQ ID NO: 142 - DNA, 50 bp, SARS-CoV-2
  misc_feature: U can be T or U
gcugcucgguauaugagaucucucaaagugccagcuacaguuucuguuuc

SEQ ID NO: 143 - DNA, 24 bp, SARS-CoV-2
gctgctcggtatatgagatctctc

SEQ ID NO: 144 - DNA, 16 bp, SARS-CoV-2
ctcggtatatgagatc

SEQ ID NO: 145 - DNA, 24 bp, SARS-CoV-2
ucucaaagugccagcuacaguuuc

SEQ ID NO: 146 - DNA, 14 bp, SARS-CoV-2
gugccagcuacagu

SEQ ID NO: 147 - DNA, 49 bp, SARS-CoV-2
gaagtaagataaccattatacgctgtaacagcatcaggtgaagaaacag

SEQ ID NO: 148 - DNA, 37 bp, SARS-CoV-2
ccattatacgctgtaacagcatcaggtgaagaaacag

SEQ ID NO: 149 - DNA, 37 bp, SARS-CoV-2
gaagtaagataaccattatacgctgtaacagcatcag

SEQ ID NO: 150 - DNA, 16 bp, SARS-CoV-2
gtaacagcatcaggtg

SEQ ID NO: 151 - DNA, 19 bp, SARS-CoV-2
gtaacagcatcaggtgaag

SEQ ID NO: 152 - DNA, 14 bp, SARS-CoV-2
gctgtaacagcatc

SEQ ID NO: 153 - DNA, 11 bp, SARS-CoV-2
cattatacgct

SEQ ID NO: 154 - DNA, 56 bp, SARS-CoV-2
  misc_feature: U can be T or U
cagggugcuguagacauaaacaagcuuugugaagaaaugcuggacaacagggcaac

SEQ ID NO: 155 - DNA, 27 bp, SARS-CoV-2
cagggtgctgtagacataaacaagctt

SEQ ID NO: 156 - DNA, 18 bp, SARS-CoV-2
ggtgctgtagacataaac

SEQ ID NO: 157 - DNA, 20 bp, SARS-CoV-2
ctgtagacataaacaagctt

SEQ ID NO: 158 - DNA, 33 bp, SARS-CoV-2
gcuuugugaagaaaugcuggacaacagggcamm

SEQ ID NO: 159 - DNA, 22 bp, SARS-CoV-2
gugaagaaaugcuggacaacag

SEQ ID NO: 160 - DNA, 73 bp, SARS-CoV-2
cttgagcagtagcaaaagctgcatatgatggaagggaactaaactctgaggctatagcttgtaagkktgccct

SEQ ID NO: 161 - DNA, 44 bp, SARS-CoV-2
cttgagcagtagcaaaagctgcatatgatggaagggaactaaac

SEQ ID NO: 162 - DNA, 33 bp, SARS-CoV-2
aaactctgaggctatagcttgtaagkktgccct

SEQ ID NO: 163 - DNA, 18 bp, SARS-CoV-2
ctatagcttgtaagkktg

SEQ ID NO: 164 - DNA, 13 bp, SARS-CoV-2
ctatagcttgtaa

SEQ ID NO: 165 - DNA, 18 bp, SARS-CoV-2
gaggctatagcttgtaag

SEQ ID NO: 166 - DNA, 19 bp, SARS-CoV-2
aaaagctgcatatgatgga

SEQ ID NO: 167 - DNA, 50 bp, Artificial Sequence
  Synthetic oligonucleotide
aatttaatacgactcactatagggagagagttgaaaggcacatttggttg

SEQ ID NO: 168 - DNA, 60 bp, Artificial Sequence
  Synthetic oligonucleotide
aatttaatacgactcactatagggagagagagttgaaaggcacatttggttgcattcatt

SEQ ID NO: 169 - DNA, 57 bp, Artificial Sequence
  Synthetic oligonucleotide
aatttaatacgactcactatagggagagagagttgaaaggcacatttggttgcattc

SEQ ID NO: 170 - DNA, 51 bp, Artificial Sequence
  Synthetic oligonucleotide
aatttaatacgactcactatagggagagtaacagcatcaggtgaagaaaca

SEQ ID NO: 171 - DNA, 52 bp, Artificial Sequence
  Synthetic oligonucleotide
aatttaatacgactcactatagggagactgtaacagcatcaggtgaagaaac

SEQ ID NO: 172 - DNA, 53 bp, Artificial Sequence
  Synthetic oligonucleotide
aatttaatacgactcactatagggagagctgtaacagcatcaggtgaagaaac

SEQ ID NO: 173 - DNA, 61 bp, Artificial Sequence
  Synthetic oligonucleotide
aatttaatacgactcactatagggagacattatacgctgtaacagcatcaggtgaagaaac

SEQ ID NO: 174 - DNA, 55 bp, Artificial Sequence
  Synthetic oligonucleotide
aatttaatacgactcactatagggagatacgctgtaacagcatcaggtgaagaaa

SEQ ID NO: 175 - DNA, 58 bp, Artificial Sequence
  Synthetic oligonucleotide
aatttaatacgactcactatagggagaccattatacgctgtaacagcatcaggtgaag

SEQ ID NO: 176 - DNA, 54 bp, Artificial Sequence
  Synthetic oligonucleotide
aatttaatacgactcactatagggagacattatacgctgtaacagcatcaggtg

SEQ ID NO: 177 - DNA, 49 bp, Artificial Sequence
  Synthetic oligonucleotide
aatttaatacgactcactatagggagacattatacgctgtaacagcatc

SEQ ID NO: 178 - DNA, 55 bp, Artificial Sequence
  Synthetic oligonucleotide
aatttaatacgactcactatagggagagctgtaacagcatcaggtgaagaaacag

SEQ ID NO: 179 - DNA, 51 bp, Artificial Sequence
  Synthetic oligonucleotide
aatttaatacgactcactatagggagagaagtaagataaccattatacgct

SEQ ID NO: 180 - DNA, 64 bp, Artificial Sequence
  Synthetic oligonucleotide
aatttaatacgactcactatagggagagaagtaagataaccattatacgctgtaacagcatcag

SEQ ID NO: 181 - DNA, 62 bp, Artificial Sequence
  Synthetic oligonucleotide
aatttaatacgactcactatagggagagaagtaagataaccattatacgctgtaacagcatc

SEQ ID NO: 182 - DNA, 61 bp, Artificial Sequence
  Synthetic oligonucleotide
aatttaatacgactcactatagggagaagcaaaagctgcatatgatggaagggaactaaac

SEQ ID NO: 183 - DNA, 55 bp, Artificial Sequence
  Synthetic oligonucleotide
aatttaatacgactcactatagggagagcaaaagctgcatatgatggaagggaac

SEQ ID NO: 184 - DNA, 62 bp, Artificial Sequence
  Synthetic oligonucleotide
aatttaatacgactcactatagggagacttgagcagtagcaaaagctgcatatgatggaagg

SEQ ID NO: 185 - DNA, 53 bp, Artificial Sequence
  Synthetic oligonucleotide
aatttaatacgactcactatagggagagaggctatagcttgtaaggttgccct

SEQ ID NO: 186 - DNA, 58 bp, Artificial Sequence
  Synthetic oligonucleotide
aatttaatacgactcactatagggagaactctgaggctatagcttgtaaggttgccct

SEQ ID NO: 187 - DNA, 54 bp, Artificial Sequence
  Synthetic oligonucleotide
aatttaatacgactcactatagggagaactctgaggctatagcttgtaaggttg

SEQ ID NO: 188 - DNA, 56 bp, Artificial Sequence
  Synthetic oligonucleotide
aatttaatacgactcactatagggagaaaactctgaggctatagcttgtaaggttg

SEQ ID NO: 189 - DNA, 57 bp, Artificial Sequence
  Synthetic oligonucleotide
aatttaatacgactcactatagggagacagtagcaaaagctgcatatgatggaaggg

SEQ ID NO: 190 - DNA, 49 bp, Artificial Sequence
  Synthetic oligonucleotide
aatttaatacgactcactatagggagactatagcttgtaaggttgccct

SEQ ID NO: 191 - DNA, 49 bp, Artificial Sequence
  Synthetic oligonucleotide
aatttaatacgactcactatagggagagaggctatagcttgtaaggttg

SEQ ID NO: 192 - DNA, 49 bp, Artificial Sequence
  Synthetic oligonucleotide
aatttaatacgactcactatagggagactctgaggctatagcttgtaag

SEQ ID NO: 193 - DNA, 48 bp, Artificial Sequence
  Synthetic oligonucleotide
aatttaatacgactcactatagggagactctgaggctatagcttgtaa

SEQ ID NO: 194 - DNA, 51 bp, Artificial Sequence
  Synthetic oligonucleotide
aatttaatacgactcactatagggagagcaaaagctgcatatgatggaagg

SEQ ID NO: 195 - DNA, 23 bp, SARS-CoV-2
gagttgaaaggcacatttggttg

SEQ ID NO: 196 - DNA, 33 bp, SARS-CoV-2; identical to SEQ ID NO: 140

SEQ ID NO: 197 - DNA, 30 bp, SARS-CoV-2
gagagttgaaaggcacatttggttgcattc

SEQ ID NO: 198 - DNA, 24 bp, SARS-CoV-2
gtaacagcatcaggtgaagaaaca

SEQ ID NO: 199 - DNA, 25 bp, SARS-CoV-2
ctgtaacagcatcaggtgaagaaac

SEQ ID NO: 200 - DNA, 26 bp, SARS-CoV-2
gctgtaacagcatcaggtgaagaaac

SEQ ID NO: 201 - DNA, 34 bp, SARS-CoV-2
cattatacgctgtaacagcatcaggtgaagaaac

SEQ ID NO: 202 - DNA, 28 bp, SARS-CoV-2
tacgctgtaacagcatcaggtgaagaaa

SEQ ID NO: 203 - DNA, 31 bp, SARS-CoV-2
ccattatacgctgtaacagcatcaggtgaag

SEQ ID NO: 204 - DNA, 27 bp, SARS-CoV-2
cattatacgctgtaacagcatcaggtg

SEQ ID NO: 205 - DNA, 22 bp, SARS-CoV-2
cattatacgctgtaacagcatc

SEQ ID NO: 206 - DNA, 28 bp, SARS-CoV-2
gctgtaacagcatcaggtgaagaaacag

SEQ ID NO: 207 - DNA, 24 bp, SARS-CoV-2
gaagtaagataaccattatacgct

SEQ ID NO: 208 - DNA, 37 bp, SARS-CoV-2; identical to SEQ ID NO: 149

SEQ ID NO: 209 - DNA, 35 bp, SARS-CoV-2
gaagtaagataaccattatacgctgtaacagcatc

SEQ ID NO: 210 - DNA, 34 bp, SARS-CoV-2
agcaaaagctgcatatgatggaagggaactaaac

SEQ ID NO: 211 - DNA, 28 bp, SARS-CoV-2
gcaaaagctgcatatgatggaagggaac

SEQ ID NO: 212 - DNA, 35 bp, SARS-CoV-2
cttgagcagtagcaaaagctgcatatgatggaagg

SEQ ID NO: 213 - DNA, 26 bp, SARS-CoV-2
gaggctatagcttgtaaggttgccct

SEQ ID NO: 214 - DNA, 31 bp, SARS-CoV-2
actctgaggctatagcttgtaaggttgccct

SEQ ID NO: 215 - DNA, 27 bp, SARS-CoV-2
actctgaggctatagcttgtaaggttg

SEQ ID NO: 216 - DNA, 29 bp, SARS-CoV-2
aaactctgaggctatagcttgtaaggttg

SEQ ID NO: 217 - DNA, 30 bp, SARS-CoV-2
cagtagcaaaagctgcatatgatggaaggg

SEQ ID NO: 218 - DNA, 22 bp, SARS-CoV-2
ctatagcttgtaaggttgccct

SEQ ID NO: 219 - DNA, 22 bp, SARS-CoV-2
gaggctatagcttgtaaggttg

SEQ ID NO: 220 - DNA, 22 bp, SARS-CoV-2
ctctgaggctatagcttgtaag

SEQ ID NO: 221 - DNA, 21 bp, SARS-CoV-2
ctctgaggctatagcttgtaa

SEQ ID NO: 222 - DNA, 24 bp, SARS-CoV-2
gcaaaagctgcatatgatggaagg

SEQ ID NO: 223 - DNA, 57 bp, Artificial Sequence
  Synthetic oligonucleotide
caaguaguggcaccuucuuuaguctttaaaaaaaaaaaaaaaaaaaaaaaaaaaaaa

SEQ ID NO: 224 - DNA, 55 bp, Artificial Sequence
  Synthetic oligonucleotide
gguggaaugugguaggauuacutttaaaaaaaaaaaaaaaaaaaaaaaaaaaaaa

SEQ ID NO: 225 - DNA, 56 bp, Artificial Sequence
  Synthetic oligonucleotide
ggaaagcaaaacagaaaguagugtttaaaaaaaaaaaaaaaaaaaaaaaaaaaaaa

SEQ ID NO: 226 - DNA, 55 bp, Artificial Sequence
  Synthetic oligonucleotide
gccugcucauaagcuucuugagtttaaaaaaaaaaaaaaaaaaaaaaaaaaaaaa

SEQ ID NO: 227 - DNA, 55 bp, Artificial Sequence
  Synthetic oligonucleotide
caacagccugcucauaagcuucuutaaaaaaaaaaaaaaaaaaaaaaaaaaaaaa

SEQ ID NO: 228 - DNA, 24 bp, SARS-CoV-2
caaguaguggcaccuucuuuaguc

SEQ ID NO: 229 - DNA, 22 bp, SARS-CoV-2
gguggaaugugguaggauuacu

SEQ ID NO: 230 - DNA, 22 bp, SARS-CoV-2
gccugcucauaagcuucuugag

SEQ ID NO: 231 - DNA, 24 bp, SARS-CoV-2
caacagccugcucauaagcuucuu

SEQ ID NO: 232 - DNA, 29 bp, Artificial Sequence
  Synthetic oligonucleotide
cgaucugucuauccaguugcgucacaucg

SEQ ID NO: 233 - DNA, 29 bp, Artificial Sequence
  Synthetic oligonucleotide
gaucugucuauccaguugcgucaccgauc

SEQ ID NO: 234 - DNA, 28 bp, Artificial Sequence
  Synthetic oligonucleotide
cgaucugucuauccaguugcgucgaucg

SEQ ID NO: 235 - DNA, 26 bp, Artificial Sequence
  Synthetic oligonucleotide
gcucaaagugccagcuacaguugagc

SEQ ID NO: 236 - DNA, 24 bp, Artificial Sequence
  Synthetic oligonucleotide
cucaaagugccagcuacaguugag

SEQ ID NO: 237 - DNA, 26 bp, Artificial Sequence
  Synthetic oligonucleotide
caaagugccagcuacaguuuccuuug

SEQ ID NO: 238 - DNA, 28 bp, Artificial Sequence
  Synthetic oligonucleotide
caaagugccagcuacaguuucugcuuug

SEQ ID NO: 239 - DNA, 24 bp, Artificial Sequence
  Synthetic oligonucleotide
agugccagcuacaguuucugcacu

SEQ ID NO: 240 - DNA, 27 bp, Artificial Sequence
  Synthetic oligonucleotide
gugccagcuacaguuucuguuucgcac

SEQ ID NO: 241 - DNA, 31 bp, SARS-CoV-2
gcuuugugaagaaaugcuggacaacagaagc

SEQ ID NO: 242 - DNA, 29 bp, SARS-CoV-2
ugugaagaaaugcuggacaacagggcaca

SEQ ID NO: 243 - DNA, 26 bp, SARS-CoV-2
gugaagaaaugcuggacaacagucac

SEQ ID NO: 244 - DNA, 23 bp, SARS-CoV-2
cgaucugucuauccaguugcguc

SEQ ID NO: 245 - DNA, 25 bp, SARS-CoV-2
cgaucugucuauccaguugcgucac

SEQ ID NO: 246 - DNA, 25 bp, SARS-CoV-2
gaucugucuauccaguugcgucacc

SEQ ID NO: 247 - DNA, 21 bp, SARS-CoV-2
gcucaaagugccagcuacagu

SEQ ID NO: 248 - DNA, 20 bp, SARS-CoV-2
cucaaagugccagcuacagu

SEQ ID NO: 249 - DNA, 21 bp, SARS-CoV-2
caaagugccagcuacaguuuc

SEQ ID NO: 250 - DNA, 23 bp, SARS-CoV-2
caaagugccagcuacaguuucug

SEQ ID NO: 251 - DNA, 20 bp, SARS-CoV-2
agugccagcuacaguuucug

SEQ ID NO: 252 - DNA, 23 bp, SARS-CoV-2
gugccagcuacaguuucuguuuc

SEQ ID NO: 253 - DNA, 27 bp, SARS-CoV-2
gcuuugugaagaaaugcuggacaacag

SEQ ID NO: 254 - DNA, 25 bp, SARS-CoV-2
ugugaagaaaugcuggacaacaggg

SEQ ID NO: 255 - DNA, 22 bp, SARS-CoV-2
gugaagaaaugcuggacaacag

SEQ ID NO: 256 - DNA, 23 bp, SARS-CoV-2
ggaaagcaaaacagaaaguagug

SEQ ID NO: 257 - DNA, 28 bp, Artificial Sequence
  Synthetic oligonucleotide
cgcucaaagugccagcuacaguugagcg

SEQ ID NO: 258 - DNA, 22 bp, SARS-CoV-2
cgcucaaagugccagcuacagu

SEQ ID NO: 259 - RNA, 1038 nt, SARS-CoV-2
gggcgaauuggguaccgggcccccccucgaggucgacgguaucgauaagcuugauaucgaauuccugcagcccgggggauccagcuuggcacugauccuuaugaagauuuucaagaaaacuggaacacuaaacauagcagugguguuacccgugaacucaugcgugagcuuaacggaggggcauacacucgcuaugucgauaacaacuucuguggcccugauggcuacccucuugagugcauuaaagaccuucuagcacgugcugguaaagcuucaugcacuuuguccgaacaacuggacuuuauugacacuaagagggguguauacugcugccgugaacaugagcaugaaauugcuugguacacggaacguucugaaaagagcuaugaauugcagacaccuuuugaaauuaaauuggcaaagaaauuugacaccuucaauggggaauguccaaauuuuguauuucccuuaaauuccauaaucaagacuauucaaccaaggguugaaaagaaaaagcuugauggcuuuauggguagaauucgaucugucuauccaguugcgucaccaaaugaaugcaaccaaaugugccuuucaacucucaugaagugugaucauuguggugaaacuucauggcagacgggcgauuuuguuaaagccacuugcgaauuuuguggcacugagaauuugacuaaagaaggugccacuacuugugguuacuuaccccaaaaugcuguuguuaaaauuuauuguccagcaugucacaauucagaaguaggaccugagcauagucuugccgaauaccauaaugaaucuggcuugaaaaccauucuucguaaggguggucgcacuauugccuuuggaggcuguguguucucuuauguugguugccauaacaagugugccuauuggguuccacgugcuagcgcuaacauagguuguaaccauacagguguuguuggagaagguuccgaaggucuuaaugacaaccuucuugaaauacuccaaaaagagaaagucaacaucaauauuguugcggcc

SEQ ID NO: 260 - RNA, 1162 nt, SARS-CoV-2
gggcgaauuggguaccgggcccccccucgaggucgacgguaucgauaagcuugauaucgaauuccugcagcccgggggaucccaguaaaacaacuguagcgucacuuaucaacacacuuaacgaucuaaaugaaacucuuguuacaaugccacuuggcuauguaacacauggcuuaaauuuggaagaagcugcucgguauaugagaucucucaaagugccagcuacaguuucuguuucuucaccugaugcuguuacagcguauaaugguuaucuuacuucuucuucuaaaacaccugaagaacauuuuauugaaaccaucucacuugcugguuccuauaaagauugguccuauucuggacaaucuacacaacuagguauagaauuucuuaagagaggugauaaaaguguauauuacacuaguaauccuaccacauuccaccuagauggugaaguuaucaccuuugacaaucuuaagacacuucuuucuuugagagaagugaggacuauuaagguguuuacaacaguagacaacauuaaccuccacacgcaaguuguggacaugucaaugacauauggacaacaguuugguccaacuuauuuggauggagcugauguuacuaaaauaaaaccucauaauucacaugaagguaaaacauuuuauguuuuaccuaaugaugacacucuacguguugaggcuuuugaguacuaccacacaacugauccuaguuuucuggguagguacaugucagcauuaaaucacacuaaaaaguggaaauacccacaaguuaaugguuuaacuucuauuaaaugggcagauaacaacuguuaucuugccacugcauuguuaacacuccaacaaauagaguugaaguuuaauccaccugcucuacaagaugcuuauuacagagcaagggcuggugaagcugcuaacuuuugugcacuuaucuuagccuacuguaauaagacaguaggugaguuaggugauguuagagaaacaaugaguuacuuguuucaacaugccaauuuagauucuugcaaaagagucuugaacgugguguguaaaacuuguggacaacagcagacaacccuuaaggguguagaagcuguuauguacaugggcacacuuucuuaugaacaauuuaagaaagguguucagcggcc

SEQ ID NO: 261 - RNA, 371 nt, SARS-CoV-2
gggcgaauuggguaccgggcccccccucgaggucgacgguaucgauaagcuugauaucgaauucuuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaaugcggcc

SEQ ID NO: 262 - RNA, 552 nt, Influenza A (H1N1)
gggcgaauuggguaccgggcccccccucgaggucgacgguaucgauaagcuuaucugucugugagaccgaugcugugaaucagcaaucuguucacaaguggcacacacuagaccaaaagcagcuucuguggucacuguucccauccuguuguauaugaggcccaugcaacuggcaagugcaccaguugaauagcuuagugacaccuccuuggccccauggaacguuauuucucuuuugagcuucuuguauaguuuaacugcucuauccauguuguucggguccccauucccauuuagggcauuuuggacaaagcgucuacgcugcaguccucgcucacugggcacggugagcgugaacacaaauccuaaaauucccuuagucagaggugacaagauuggucuugucuuuagccauuccaugagagccucaagaucuguguucuuuccugcaaagacacuuuccagucucugcgcgaucucggcuuugagggggccugacgggaugauagaaagaacguacguuucgaccucgguuagaagacucaugcggcc

SEQ ID NO: 263 - RNA, 552 nt, Influenza A (H3N2)
gggcgaauuggguaccgggcccccccucgaggucgacgguaucgauaagcuuaucugccuaugagaccugugcugggagucagcaaucugcucacauguugcacauaccaggccaaaugccacuucagugguuacagcccccauccuauuguauaugaggcccaugcaacuggcaagugcaccagcggaauaacugagagcuauuucuuuggccccauggaacguuaucucucucuuaaguuuccuauacaguuuaacugcuuuguccauguuauuugggucuccauucccauugagggcauuuuggacaaagcgucuacgcugcaguccucgcucacugggcacggugagcgugaacacaaaccccaaaauccccuuagucagaggugacagaauuggucuugucuuuagccauuccaugagagccucaagaucuguguuuuucccagcaaagacaucuucaagucucugcgcgaucucggcuuugagggggccugauggaacgauagagagaacauacguuucgaccucgguuagaaggcucaugcggcc

SEQ ID NO: 264 - RNA, 805 nt, Influenza B
gggcgaauuggguaccgggcccccccucgaggucgacgguaucgauaagcuuuuauagguauuucuucacaagagcugaauuucccauagagcucugcuuuagcacuuccauuacauccuuugcaauuccuuccccauucuucugacuugccccaagggaucucaauacuccaauguugcuuugcaguucuucugccaguuuuuggacgucuucuccuuuucccauuccauucauuguuuuugcuguguucauagcugagaccaucugcauuucccgucucacuccuggcacugaggaucucgcugcucugcuaugagcucugugugaaugugaugcuuguuuuucgcacaaagcacagagcguuccuaguuuuacuugcauugaauaauuuccaggauucagguacaugaccaugagacaauauaguagcgcuuagcuuucauggccuucugcuauuucaaaugcuucaugaaagcucacacauuuucucauuuuucucucagccagagucaggcccuucuuuuuuguugcuguuguucccauucccgauaagggcucugugaugaaucuucuuuuucuuuccuggucuuuggguuuuaaaaagcagauagaggcaccaauuagugcuuucuguauaucaguuaagcaucuuuuguuuuuuauccauuccaaggcagagucuaggucaaauucuuucccaccgaaccaacaguguaauuuuucugcuaguucugcuuugccuucuccaucuucugucaaugaaagcagguaggcaauugugucuccaaacagcgacaugcggcc

SEQ ID NO: 265 - RNA, 315 nt, Influenza B
gggcgaauuggguaccgggcccccccucgaggucgacgguaucgauaagcuucuaauugucucccucuucuggugauaaucggugcucuugaccaaauugggauaagacucccaccgcaguuucagcugcucgaauuggcuuugaauguccuucauuaagacgcucgaagagugaauugaggauccgauggccaucuucuucauccuccacuguaagaucaucaguagcaacaaguuuagcaacaagccuuccacucuggucauaugcauucaaucuaugcagaguugauaaggacuuguauccauugggcggcc

SEQ ID NO: 266 - RNA, 30 nt, Artificial Sequence
  Synthetic oligonucleotide
gcgaucugucuauccaguugcgucgaucgc

SEQ ID NO: 267 - RNA, 28 nt, Artificial Sequence
  Synthetic oligonucleotide
gacgccgaucugucuauccaguugcguc

SEQ ID NO: 268 - RNA, 30 nt, Artificial Sequence
  Synthetic oligonucleotide
cgaucugucuauccaguugcguccagaucg

SEQ ID NO: 269 - RNA, 32 nt, Artificial Sequence
  Synthetic oligonucleotide
cgcgaucugucuauccaguugcgucgaucgcg

SEQ ID NO: 270 - RNA, 31 nt, Artificial Sequence
  Synthetic oligonucleotide
gcgaucugucuauccaguugcgucagaucgc

SEQ ID NO: 271 - RNA, 35 nt, Artificial Sequence
  Synthetic oligonucleotide
  misc_feature (1)..(5): n is a, c, g, or u
  misc_feature (29)..(35): n is a, c, g, or u
nnnnncgaucugucuauccaguugcgucnnnnnnn

SEQ ID NO: 272 - RNA, 35 nt, Artificial Sequence
  Synthetic oligonucleotide
  misc_feature (1)..(3): n is a, c, g, or u
nnnsscgaucugucuauccaguugcgucvrdhbss

SEQ ID NO: 273 - RNA, 35 nt, Artificial Sequence
  Synthetic oligonucleotide
sassscgaucugucuauccaguugcgucvrdhbss

SEQ ID NO: 274 - RNA, 34 nt, Artificial Sequence
  Synthetic oligonucleotide
  misc_feature (1)..(5): nucleotide is independently present or absent
  misc_feature (29)..(34): nucleotide is independently present or absent
nnnnncgaucugucuauccaguugcgucnnnnns

SEQ ID NO: 275 - RNA, 35 nt, Artificial Sequence
  Synthetic oligonucleotide
  misc_feature (1)..(5): nucleotide is independently present or absent
  misc_feature (29)..(33): nucleotide is independently present or absent
nnnnncgaucugucuauccaguugcgucnnnnnss